SEQUENCE LISTING (222 sequences)

SEQ ID NO: 1 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, synthetic construct
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, synthetic construct
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 7 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, synthetic construct
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 12 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 13 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 15 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - DNA, 2208 bp, synthetic construct
  source 1..2208: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctagagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 17 - DNA, 255 bp, synthetic construct
  source 1..255: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcatt

SEQ ID NO: 18 - DNA, 255 bp, synthetic construct
  source 1..255: mol_type = other DNA
gattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcatt

SEQ ID NO: 19 - DNA, 255 bp, synthetic construct
  source 1..255: mol_type = other DNA
aatgactcctttcggtaagtgcagtggaagctgtacactgcccaggcaaagcgtccgggcagagtaggtgggggactcagatcccagccagtggacttagcccctgtttgctcctccgataactggggtgaccttggttaatattcaccagcagcctccccctttgcccctctggatccactgcttaaatacggacgaggacagggccctgtctcctcagcttcaggcaccaccactgacctgggacagtgaatc

SEQ ID NO: 20 - DNA, 180 bp, synthetic construct
  source 1..180: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgag

SEQ ID NO: 21 - DNA, 180 bp, synthetic construct
  source 1..180: mol_type = other DNA
ctcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 22 - DNA, 314 bp, synthetic construct
  source 1..314: mol_type = other DNA
gaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 23 - DNA, 314 bp, synthetic construct
  source 1..314: mol_type = other DNA
ccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctc

SEQ ID NO: 24 - DNA, 192 bp, synthetic construct
  source 1..192: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 25 - DNA, 192 bp, synthetic construct
  source 1..192: mol_type = other DNA
ccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggg

SEQ ID NO: 26 - DNA, 202 bp, synthetic construct
  source 1..202: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 27 - DNA, 612 bp, synthetic construct
  source 1..612: mol_type = other DNA
gcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatc

SEQ ID NO: 28 - DNA, 73 bp, synthetic construct
  source 1..73: mol_type = other DNA
ctaaaagagagaaacaattagaatcagtagtttaacacattatacacttaaaaattttatatttaccttagag

SEQ ID NO: 29 - DNA, 322 bp, synthetic construct
  source 1..322: mol_type = other DNA
gaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccacc

SEQ ID NO: 30 - DNA, 322 bp, synthetic construct
  source 1..322: mol_type = other DNA
gaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccacc

SEQ ID NO: 31 - DNA, 322 bp, synthetic construct
  source 1..322: mol_type = other DNA
gaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccacc

SEQ ID NO: 32 - DNA, 92 bp, synthetic construct
  source 1..92: mol_type = other DNA
aagaggtaagggtttaagggatggttggttggtggggtattaatgtttaattacctggagcacctgcctgaaatcactttttttcaggttgg

SEQ ID NO: 33 - DNA, 135 bp, synthetic construct
  source 1..135: mol_type = other DNA
ccccttgctccataccacccccctccaccccagagacacaaaaaattccaacacacgagacaaaattgttgttaacttgtttattaggacaaggctggtcggcactggagtggcaacttccagggccaggagagg

SEQ ID NO: 34 - DNA, 171 bp, synthetic construct
  source 1..171: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacag

SEQ ID NO: 35 - DNA, 198 bp, synthetic construct
  source 1..198: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 36 - DNA, 570 bp, synthetic construct
  source 1..570: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 37 - DNA, 570 bp, synthetic construct
  source 1..570: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctc

SEQ ID NO: 38 - DNA, 569 bp, synthetic construct
  source 1..569: mol_type = other DNA
gattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 39 - DNA, 494 bp, synthetic construct
  source 1..494: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctc

SEQ ID NO: 40 - DNA, 494 bp, synthetic construct
  source 1..494: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 41 - DNA, 516 bp, synthetic construct
  source 1..516: mol_type = other DNA
ccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 42 - DNA, 516 bp, synthetic construct
  source 1..516: mol_type = other DNA
gaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 43 - DNA, 394 bp, synthetic construct
  source 1..394: mol_type = other DNA
ccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 44 - DNA, 1128 bp, synthetic construct
  source 1..1128: mol_type = other DNA
gaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 45 - DNA, 1006 bp, synthetic construct
  source 1..1006: mol_type = other DNA
ccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 46 - DNA, 328 bp, synthetic construct
  source 1..328: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcatt

SEQ ID NO: 47 - DNA, 642 bp, synthetic construct
  source 1..642: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 48 - DNA, 253 bp, synthetic construct
  source 1..253: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgag

SEQ ID NO: 49 - DNA, 357 bp, synthetic construct
  source 1..357: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcatt

SEQ ID NO: 50 - DNA, 357 bp, synthetic construct
  source 1..357: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcatt

SEQ ID NO: 51 - DNA, 282 bp, synthetic construct
  source 1..282: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgag

SEQ ID NO: 52 - DNA, 307 bp, synthetic construct
  source 1..307: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttgg

SEQ ID NO: 53 - DNA, 307 bp, synthetic construct
  source 1..307: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttgg

SEQ ID NO: 54 - DNA, 471 bp, synthetic construct
  source 1..471: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacag

SEQ ID NO: 55 - DNA, 471 bp, synthetic construct
  source 1..471: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacag

SEQ ID NO: 56 - DNA, 322 bp, synthetic construct
  source 1..322: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatg

SEQ ID NO: 57 - DNA, 322 bp, synthetic construct
  source 1..322: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggactggtgccaccatg

SEQ ID NO: 58 - DNA, 528 bp, synthetic construct
  source 1..528: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccaccatg

SEQ ID NO: 59 - DNA, 528 bp, synthetic construct
  source 1..528: mol_type = other DNA
acctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatg

SEQ ID NO: 60 - DNA, 650 bp, synthetic construct
  source 1..650: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 61 - DNA, 772 bp, synthetic construct
  source 1..772: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 62 - DNA, 772 bp, synthetic construct
  source 1..772: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 63 - DNA, 696 bp, synthetic construct
  source 1..696: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 64 - DNA, 696 bp, synthetic construct
  source 1..696: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 65 - DNA, 1262 bp, synthetic construct
  source 1..1262: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 66 - DNA, 771 bp, synthetic construct
  source 1..771: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 67 - DNA, 650 bp, synthetic construct; identical to SEQ ID NO: 60
  source 1..650: mol_type = other DNA

SEQ ID NO: 68 - DNA, 772 bp, synthetic construct; identical to SEQ ID NO: 62
  source 1..772: mol_type = other DNA

SEQ ID NO: 69 - DNA, 696 bp, synthetic construct; identical to SEQ ID NO: 63
  source 1..696: mol_type = other DNA

SEQ ID NO: 70 - DNA, 696 bp, synthetic construct; identical to SEQ ID NO: 64
  source 1..696: mol_type = other DNA

SEQ ID NO: 71 - DNA, 771 bp, synthetic construct
  source 1..771: mol_type = other DNA
gattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 72 - DNA, 569 bp, synthetic construct
  source 1..569: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 73 - DNA, 3375 bp, synthetic construct
  source 1..3375: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcacgtgccgcggccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaaagaaagagaagaggcagcggagaaggcagaggcagcctgctgacctgcggcgacgtcgaagagaaccccggccctatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 74 - DNA, 3863 bp, synthetic construct
  source 1..3863: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcacgtgccgcggccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaactagtgccaccatgggctggtcctgcatcatcctgttcctggtggccaccgccacaggcgtgcacagccaagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaataaaacgttcctcgaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaaggtccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 75 - DNA, 4483 bp, synthetic construct
  source 1..4483: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 76 - DNA, 1383 bp, synthetic construct
  source 1..1383: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 77 - DNA, 1262 bp, synthetic construct; identical to SEQ ID NO: 65
  source 1..1262: mol_type = other DNA

SEQ ID NO: 78 - DNA, 1308 bp, synthetic construct
  source 1..1308: mol_type = other DNA
gattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatc

SEQ ID NO: 79 - DNA, 828 bp, synthetic construct
  source 1..828: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttgg

SEQ ID NO: 80 - DNA, 950 bp, synthetic construct
  source 1..950: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttgg

SEQ ID NO: 81 - DNA, 950 bp, synthetic construct
  source 1..950: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttgg

SEQ ID NO: 82 - DNA, 874 bp, synthetic construct
  source 1..874: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttgg

SEQ ID NO: 83 - DNA, 874 bp, synthetic construct
  source 1..874: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttgg

SEQ ID NO: 84 - DNA, 1440 bp, synthetic construct
  source 1..1440: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttgg

SEQ ID NO: 85 - DNA, 949 bp, synthetic construct
  source 1..949: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttgg

SEQ ID NO: 86 - DNA, 828 bp, synthetic construct
  source 1..828: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttgg

SEQ ID NO: 87 - DNA, 950 bp, synthetic construct
  source 1..950: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttgg

SEQ ID NO: 88 - DNA, 874 bp, synthetic construct
  source 1..874: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttgg

SEQ ID NO: 89 - DNA, 874 bp, synthetic construct
  source 1..874: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttgg

SEQ ID NO: 90 - DNA, 1113 bp, synthetic construct
  source 1..1113: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacag

SEQ ID NO: 91 - DNA, 1113 bp, synthetic construct
  source 1..1113: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacag

SEQ ID NO: 92 - DNA, 949 bp, synthetic construct; identical to SEQ ID NO: 85
  source 1..949: mol_type = other DNA

SEQ ID NO: 93 - DNA, 1113 bp, synthetic construct
  source 1..1113: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacag

SEQ ID NO: 94 - DNA, 828 bp, synthetic construct; identical to SEQ ID NO: 79
  source 1..828: mol_type = other DNA

SEQ ID NO: 95 - DNA, 1725 bp, synthetic construct
  source 1..1725: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacag

SEQ ID NO: 96 - DNA, 1603 bp, synthetic construct
  source 1..1603: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcgaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacag

SEQ ID NO: 97 - DNA, 1650 bp, synthetic construct
  source 1..1650: mol_type = other DNA
ctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacag

SEQ ID NO: 98 - DNA, 878 bp, synthetic construct
  source 1..878: mol_type = other DNA
catggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatg

SEQ ID NO: 99 - DNA, 1000 bp, synthetic construct
  source 1..1000: mol_type = other DNA
catggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatg

SEQ ID NO: 100 - DNA, 1000 bp, synthetic construct
  source 1..1000: mol_type = other DNA
catggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatg

SEQ ID NO: 101 - DNA, 924 bp, synthetic construct
  source 1..924: mol_type = other DNA
catggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatg

SEQ ID NO: 102 - DNA, 924 bp, synthetic construct
  source 1..924: mol_type = other DNA
catggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatg

SEQ ID NO: 103 - DNA, 1490 bp, synthetic construct
  source 1..1490: mol_type = other DNA
catggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatg

SEQ ID NO: 104 - DNA, 997 bp, synthetic construct
  source 1..997: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaccatggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatg

SEQ ID NO: 105 - DNA, 872 bp, synthetic construct
  source 1..872: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatg

SEQ ID NO: 106 - DNA, 994 bp, synthetic construct
  source 1..994: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatg

SEQ ID NO: 107 - DNA, 918 bp, synthetic construct
  source 1..918: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatg

SEQ ID NO: 108 - DNA, 918 bp, synthetic construct
  source 1..918: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatg

SEQ ID NO: 109 - DNA, 1199 bp, synthetic construct
  source 1..1199: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccaccatg

SEQ ID NO: 110 - DNA, 1199 bp, synthetic construct
  source 1..1199: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccaccatg

SEQ ID NO: 111 - DNA, 993 bp, synthetic construct
  source 1..993: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatg

SEQ ID NO: 112 - DNA, 1199 bp, synthetic construct
  source 1..1199: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatg

SEQ ID NO: 113 - DNA, 872 bp, synthetic construct
  source 1..872: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggactggtgccaccatg

SEQ ID NO: 114 - DNA, 1811 bp, synthetic construct
  source 1..1811: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatg

SEQ ID NO: 115 - DNA, 1689 bp, synthetic construct
  source 1..1689: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcgaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatg

SEQ ID NO: 116 - DNA, 1736 bp, synthetic construct
  source 1..1736: mol_type = other DNA
catggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatg

SEQ ID NO: 117 - DNA, 3388 bp, synthetic construct
  source 1..3388: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 118 - DNA, 3510 bp, synthetic construct
  source 1..3510: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 119 - DNA, 3510 bp, synthetic construct
  source 1..3510: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 120 - DNA, 3434 bp, synthetic construct
  source 1..3434: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 121 - DNA, 3434 bp, synthetic construct
  source 1..3434: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 122 - DNA, 4000 bp, synthetic construct
  source 1..4000: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 123 - DNA, 3471 bp, synthetic construct
  source 1..3471: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 124 - DNA, 3471 bp, synthetic construct
  source 1..3471: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcattttcctagggataatgacaggctcttctgtgtgtagtggttgtgcagagcctcatgcatgacagagcaggaaaagacattgccctcctgccatctacttttatctacagtcaacctggaatacaggaagaagctgccatcagagtccagcactgggggggtggttttatagttgttttctggctgcccattgctctcccattccacagcaatgtcactagggtagaagcccttcacaagacaagtaagagaaacctggttcttggtcatctcttcttgagaagggggcagggtatacacctgaggttctctaggttgacctttagctttgcttatggtcttctcaatggagctggggagtcccttgttgctcactttacacttgtactctttgccattcagccagtcctggtggagcactgtgaggacacttaccaccctataggtgctgttgaactgctcctccctgggtttagtcttggcattatggacctccactccatccacataccaattaaactgcacctctggatcttcctgggaaacatccaccacaacacaggtgacttctggtgtcctggagatcattagtgtatccttgggctttggggggaagaggaacacactgggcccagcaacagggggggctgggcaaggaggacactccacacagcactttctttcaactgtcttgtccaccttggtgttagaaggcttgtggtccacattgcaggtgtaagtctgggtcccaaaattggatgaggggactgttacaactgaactcaaggaataaagacctgagctctgcagaacagctggaaaggtatgcactccagaggtcagggccccagaattccaggatacagtcacaggctcagggaagtagtctttgacaaggcaccccagggctgcagtggattctgaagttgatctgctgcagggtgccaggggaaacacagaggggccttttgttgaggcagaggaaactgtcaccaaagtgccttgtccccacacatcaaagtaccaatttgggctggatccaaagaaatatctggcacagtagtagactgctgtgtcttcagacctcaaggaactcagctccatgtagactgtgctggtactggtgtccctagtcatggtcactctgtccttgaagttctcagtatactctgtgctgcctgaacctgggaggatctcccccatccactccaggccctggccaggggcctgcctgacccactggatccagtagttgctgaagatgtagccagatgccttgcagctgactttcactgaggcacctggcttctttacttcagccccactctgcaccagctggacttggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacccagagccccagcagcctatcagcttctgtaggggacagggtcaccatcacctgtggagcatcagagaacatatatggggccctgaactggtaccagcagaaaccaggcaaggcccccaagctgctgatttatggtgccaccaacctggcagatggtgttccttccaggtttagtggctctggaagtggaacagacttcaccctcaccatttccagcctgcagccagaagattttgccacttactactgccagaatgtgctgaatactcctttgacatttggccaagggactaaagtggagatcaagaggacagtagctgctccttctgtcttcatcttccccccctctgatgaacagctgaagtctggcacagccagtgtggtgtgcctgctcaacaacttctacccaagagaagcaaaagtgcagtggaaggtggacaatgcccttcaatctgggaattcccaggagagtgtcacagagcaggacagcaaagattccacctacagcctctccagcacacttactttatcaaaagctgattatgagaagcacaaggtctatgcctgtgaagttactcatcaaggcttgagctcccctgtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 125 - DNA, 3471 bp, synthetic construct
  source 1..3471: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttccccaggctgaggcttaagctcttctgagtgtaatggttgtggagtgcttcatgcatgacactgcaggagaagacattgccctcctgccatctacttttgtcaactgttagcctgctatacaggaagaagctgccatcagaatcaagcacaggaggggttgtcttataattattttcaggttgtccattggactcccactctacagcaatgtctgatggatagaagcctttgaccaggcaggttagagacacctggttctttgtcatctcttcttgggagggaggcaatgtgtacacttggggctctctgggctgtcctttggccttgcttatagtcttctcaatgctgctgggcaggcctttgttggaaaccttacatttatactccttgccattcagccagtcctggtgcagcacagtgagcacagacactaccctgtaggtggagttaaactgctcttccctgggcttggtttttgcattgtgcacttccactccatccacataccaattaaactgaacttctgggtcctcctgactgacatctaccaccacacaagtgacctctggagtcctggagatcatcagtgtgtcttttggcttgggggggaagagaaagactgatggtccagccactggaggggctggacaagggggacattctacacagcacttcctttcaacagtcttgtccactttggtgttgctaggcttgtggtccacattgcaagtataggtctgggtcccaaaattactggaaggaacagtaactactgaggataatgagtaaagccctgaggactgcaggacagctgggaaggtatgcacacctgaggtgagggccccagagttccaagagactgtaacaggctcagggaagtagtccttcacaaggcagcccaaggcagcagtgctctcacttgttgatctggagcagggtgccaggggaaaaacagagggccccttggttgaggcactggagactgtgaccagggtgccctggccccacacatcaaagtaccagttggggctagagccaaaaaaatatctggcacagtagtagactgctgtatcttcagatctgagagaggacagctccatgtagactgtggaagtgctggtgtctctggtcatggtcactctatccttaaagttctctgtatattctgtgcttccagaacctggcaagatctcccccatccattccaatccttgccctggggcttgcctgacccattggatccagtagttactgaaaatgtagccagatgctttgcaggagacctttacactggctcctggcttcttcacctcagctccactctgcaccagctgcacttggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacccagagcccctcttccttatcagcatcagttggagatagagtcaccattacttgtggggcttcagaaaatatatatggtgccctcaactggtaccagcagaagccaggcaaagcccccaagctgctcatttatggggccaccaacctggctgatggtgtgccctccaggttttctggctctggaagtggaacagacttcaccctaaccatctcctcccttcaacctgaagattttgcaacttactactgccagaatgtgctgaatacaccacttacatttggccaaggcaccaaagttgagatcaagaggactgtagctgctcctagtgtgttcatcttccctccatctgatgaacagctgaaatcaggcactgcatctgtggtctgcctgctgaacaacttctaccccagggaggccaaggtccagtggaaggtggacaatgccctgcagagtgggaatagccaggagtctgttacagagcaggactccaaggacagcacatacagcctctcctctactttgaccttgtccaaagcagattatgagaagcacaaagtatatgcctgtgaggtgacacatcaagggctcagcagccctgtgaccaaatccttcaacagaggagaatgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 126 - DNA, 3471 bp, synthetic construct
  source 1..3471: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcacttccctaatgagagggataagctcttctgggtgtagtggttgtggagggcttcatgcatcacactgcaggagaagacatttccttcctgccacctggatttatccacagtcaacctgctgtacaggaagaagcttccatcactgtccagcactggtggtgtggttttgtagttgttttcaggctgcccattactctcccattcaactgcaatatctgaaggataaaagcctttcacaagacaagttagagacacttgatttttggtcatctcctcctgggaaggtggcagtgtgtagacctggggctctctgggctggccctttgctttggagatggtcttttcaatggaggagggtaggcccttgttggacactttgcacttgtactccttgccattcagccagtcctggtgcaagacagtgagcacactgactactctataggtgctgttgaactgctcttctctaggctttgtcttggcattatgcacctccaccccatccacataccaattaaactgaacttctggatcttcttgagacacatcaaccacaacacaggtgacctctggggttcttgaaatcatcagagtatccttgggtttgggggggaagagaaatacactgggcccagcaactgggggggcagggcagggtggacattccacacagcatttcctttctactgttttgtccaccttggtgttgctgggcttgtggtccacattgcaggtatatgtctgagtgccaaagttagagcttggaactgtcactactgaactaagtgaataaagcccagagctctgcaggacagctggaaaagtatgaacaccagaggtgagagcacctgaattccagcttacagtcacaggctcagggaagtagtccttcaccaggcagcccagggctgcagtactttcagaagtggacctggaacatggagccaaaggaaaaacagaaggtcctttagtgctagctgatgagacagttaccaatgtgccttgtccccagacatcaaagtaccagttgggactagatccaaaaaaatatctggcacaataatacacagctgtgtcctcagatctaagggagctcagctccatgtacactgtggaggtgctggtgtctcttgtcatggttaccctgtctttgaagttctctgtatactctgtgctgccactcccaggtaatatttctcccatccactccaggccttggccaggggcttgcctgacccactggatccagtaattactgaagatgtagccagatgccttgcagctaactttgactgaggcacctggcttcttgacttctgctccagactggaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacccaaagccccagctccttgtcagcttcagttggtgacagggtaacaattacttgtggggcttcagaaaatatttatggagcactcaactggtaccagcagaagccaggcaaggccccaaagttgttaatatatggggccacaaacctggcagatggggtaccctccaggttttctggttcaggcagtgggacagacttcaccctcaccatcagcagtcttcaaccagaagattttgccacctactactgccagaatgtgctgaatactcctcttacatttggacaaggcaccaaggtggagattaaaagaactgtggctgcacccagtgtcttcatctttcctccttctgatgaacagctgaagagtggcacagcatctgtggtgtgcctgctgaacaacttctacccaagagaggccaaggtccagtggaaagtggacaatgccctgcaaagtggaaattcccaggagtctgttacagagcaggacagcaaagattctacttacagcctaagcagcactttaactctttccaaggctgattatgaaaaacataaagtttatgcctgtgaggtgacccaccagggcctctcctctcctgtcaccaaatccttcaacagaggagaatgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 127 - DNA, 3471 bp, synthetic construct
  source 1..3471: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcattttcctaaactgagggagagactcttctgggtgtagtggttgtgcagagcttcatgcatgacagaacatgaaaagacattcccttcctgccatctggacttgtctactgtcagcctgctatacaggaagaaggaaccatctgagtctagtactggaggggtggtcttataattattttctggctgcccattactctcccattccacagcaatatctgaggggtagaagcccttcacaaggcaggtcaaggacacctggttttttgtcatttcttcttgtgatggggggagagtatagacttgtggctccctgggctggcctttggctttgctgatagtcttctctatgctggagggcaagcctttgttactgactttgcatttatactcctttccattcagccagtcctgatggaggacagtgagcacagaaacaactctataagtgctgttaaactgctcctctctaggcttggttttagcattatgaacttctactccatcaacataccaattaaattgcacctcagggtcctcctgggaaacatcaaccaccacacaggtgacttctggagtcctactaatcatgagtgtgtccttgggcttaggggggaatagaaagactgaagggcctgcaactgggggggcagggcatgggggacactcaacacagcattttctctccacagttttgtccacttttgtattgctgggcttgtggtccacattgcaagtgtaggtctgggttccaaagttgctagatggtactgtgaccacagatgacagggaataaaggccactgctctgcagtactgcagggaaggtgtgcaccccacttgtcaaagcaccactattccatgaaacagtcacaggctcaggaaaatagtctttcaccaggcagcccagggctgctgtgctctcagatgtggacctactgcagggggctaatgggaagacagatggtcctttagttgaggcagaagacactgtcaccaaggtgccctggccccagacatcaaaataccagttggggctgctgccaaagaagtatctggcacagtagtagactgctgtatcttctgacctaaggctggacagctccatgtacactgtagaggtggaggtgtctctggtcatggtcactctatccttgaagttttctgtatattcagtggagccagacccaggaagaatctcccccatccactccaggccttgcccaggggcttgtctgacccattgaatccagtagttggaaaagatgtaaccagatgccttgcagctgactttgactgaggcccctggcttcttcacttcagctccactctggaccagctgtacttggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacccagagtcccagtagtctatcagcctctgtaggggatagagttaccatcacctgtggggcttcagaaaatatatatggggccctcaactggtaccagcagaaaccaggaaaagcacccaagctcttgatttatggagccacaaatttggcagatggagtacccagcaggttttctggttctggctcaggcactgacttcaccttaactatttcctccctgcaacctgaggactttgcaacttactactgccagaatgtgctgaatacacctcttacctttggccaagggacaaaagtggagatcaagaggactgtggctgcaccatctgtcttcatttttcccccttctgatgaacagctgaagagtggtacagcttctgtggtgtgcctgctgaacaacttctacccaagagaagccaaggttcagtggaaggtggacaatgccctgcaaagtggcaacagccaggagtcagtcacagagcaggacagcaaagattccacctacagcctctccagcactcttacactttccaaagctgattatgagaagcacaaagtttatgcctgtgaggtgactcatcaaggcctgagcagccctgtcaccaagtccttcaacagaggagaatgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 128 - DNA, 3350 bp, synthetic construct
  source 1..3350: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 129 - DNA, 3472 bp, synthetic construct
  source 1..3472: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 130 - DNA, 3396 bp, synthetic construct
  source 1..3396: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 131 - DNA, 3396 bp, synthetic construct
  source 1..3396: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 132 - DNA, 3641 bp, synthetic construct
  source 1..3641: mol_type = other DNA
ccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 133 - DNA, 3641 bp, synthetic construct
  source 1..3641: mol_type = other DNA
ccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 134 - DNA, 3435 bp, synthetic construct
  source 1..3435: mol_type = other DNA
ccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 135 - DNA, 3677 bp, synthetic construct
  source 1..3677: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 136 - DNA, 3641 bp, synthetic construct
  source 1..3641: mol_type = other DNA
ccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 137 - DNA, 3350 bp, synthetic construct
  source 1..3350: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggactggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 138 - DNA, 4253 bp, synthetic construct
  source 1..4253: mol_type = other DNA
ccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 139 - DNA, 4167 bp, synthetic construct
  source 1..4167: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcgaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 140 - DNA, 4214 bp, synthetic construct
  source 1..4214: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 141 - DNA, 3796 bp, synthetic construct
  source 1..3796: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 142 - DNA, 3918 bp, synthetic construct
  source 1..3918: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 143 - DNA, 3918 bp, synthetic construct
  source 1..3918: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 144 - DNA, 3842 bp, synthetic construct
  source 1..3842: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 145 - DNA, 3842 bp, synthetic construct
  source 1..3842: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 146 - DNA, 4408 bp, synthetic construct
  source 1..4408: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 147 - DNA, 3852 bp, synthetic construct
  source 1..3852: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 148 - DNA, 3852 bp, synthetic construct
  source 1..3852: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcattttcctagggataatgacaggctcttctgtgtgtagtggttgtgcagagcctcatgcatgacagagcaggaaaagacattgccctcctgccatctacttttatctacagtcaacctggaatacaggaagaagctgccatcagagtccagcactgggggggtggttttatagttgttttctggctgcccattgctctcccattccacagcaatgtcactagggtagaagcccttcacaagacaagtaagagaaacctggttcttggtcatctcttcttgagaagggggcagggtatacacctgaggttctctaggttgacctttagctttgcttatggtcttctcaatggagctggggagtcccttgttgctcactttacacttgtactctttgccattcagccagtcctggtggagcactgtgaggacacttaccaccctataggtgctgttgaactgctcctccctgggtttagtcttggcattatggacctccactccatccacataccaattaaactgcacctctggatcttcctgggaaacatccaccacaacacaggtgacttctggtgtcctggagatcattagtgtatccttgggctttggggggaagaggaacacactgggcccagcaacagggggggctgggcaaggaggacactccacacagcactttctttcaactgtcttgtccaccttggtgttagaaggcttgtggtccacattgcaggtgtaagtctgggtcccaaaattggatgaggggactgttacaactgaactcaaggaataaagacctgagctctgcagaacagctggaaaggtatgcactccagaggtcagggccccagaattccaggatacagtcacaggctcagggaagtagtctttgacaaggcaccccagggctgcagtggattctgaagttgatctgctgcagggtgccaggggaaacacagaggggccttttgttgaggcagaggaaactgtcaccaaagtgccttgtccccacacatcaaagtaccaatttgggctggatccaaagaaatatctggcacagtagtagactgctgtgtcttcagacctcaaggaactcagctccatgtagactgtgctggtactggtgtccctagtcatggtcactctgtccttgaagttctcagtatactctgtgctgcctgaacctgggaggatctcccccatccactccaggccctggccaggggcctgcctgacccactggatccagtagttgctgaagatgtagccagatgccttgcagctgactttcactgaggcacctggcttctttacttcagccccactctgcaccagctggacttggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacccagagccccagcagcctatcagcttctgtaggggacagggtcaccatcacctgtggagcatcagagaacatatatggggccctgaactggtaccagcagaaaccaggcaaggcccccaagctgctgatttatggtgccaccaacctggcagatggtgttccttccaggtttagtggctctggaagtggaacagacttcaccctcaccatttccagcctgcagccagaagattttgccacttactactgccagaatgtgctgaatactcctttgacatttggccaagggactaaagtggagatcaagaggacagtagctgctccttctgtcttcatcttccccccctctgatgaacagctgaagtctggcacagccagtgtggtgtgcctgctcaacaacttctacccaagagaagcaaaagtgcagtggaaggtggacaatgcccttcaatctgggaattcccaggagagtgtcacagagcaggacagcaaagattccacctacagcctctccagcacacttactttatcaaaagctgattatgagaagcacaaggtctatgcctgtgaagttactcatcaaggcttgagctcccctgtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 149 - DNA, 3852 bp, synthetic construct
  source 1..3852: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttccccaggctgaggcttaagctcttctgagtgtaatggttgtggagtgcttcatgcatgacactgcaggagaagacattgccctcctgccatctacttttgtcaactgttagcctgctatacaggaagaagctgccatcagaatcaagcacaggaggggttgtcttataattattttcaggttgtccattggactcccactctacagcaatgtctgatggatagaagcctttgaccaggcaggttagagacacctggttctttgtcatctcttcttgggagggaggcaatgtgtacacttggggctctctgggctgtcctttggccttgcttatagtcttctcaatgctgctgggcaggcctttgttggaaaccttacatttatactccttgccattcagccagtcctggtgcagcacagtgagcacagacactaccctgtaggtggagttaaactgctcttccctgggcttggtttttgcattgtgcacttccactccatccacataccaattaaactgaacttctgggtcctcctgactgacatctaccaccacacaagtgacctctggagtcctggagatcatcagtgtgtcttttggcttgggggggaagagaaagactgatggtccagccactggaggggctggacaagggggacattctacacagcacttcctttcaacagtcttgtccactttggtgttgctaggcttgtggtccacattgcaagtataggtctgggtcccaaaattactggaaggaacagtaactactgaggataatgagtaaagccctgaggactgcaggacagctgggaaggtatgcacacctgaggtgagggccccagagttccaagagactgtaacaggctcagggaagtagtccttcacaaggcagcccaaggcagcagtgctctcacttgttgatctggagcagggtgccaggggaaaaacagagggccccttggttgaggcactggagactgtgaccagggtgccctggccccacacatcaaagtaccagttggggctagagccaaaaaaatatctggcacagtagtagactgctgtatcttcagatctgagagaggacagctccatgtagactgtggaagtgctggtgtctctggtcatggtcactctatccttaaagttctctgtatattctgtgcttccagaacctggcaagatctcccccatccattccaatccttgccctggggcttgcctgacccattggatccagtagttactgaaaatgtagccagatgctttgcaggagacctttacactggctcctggcttcttcacctcagctccactctgcaccagctgcacttggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacccagagcccctcttccttatcagcatcagttggagatagagtcaccattacttgtggggcttcagaaaatatatatggtgccctcaactggtaccagcagaagccaggcaaagcccccaagctgctcatttatggggccaccaacctggctgatggtgtgccctccaggttttctggctctggaagtggaacagacttcaccctaaccatctcctcccttcaacctgaagattttgcaacttactactgccagaatgtgctgaatacaccacttacatttggccaaggcaccaaagttgagatcaagaggactgtagctgctcctagtgtgttcatcttccctccatctgatgaacagctgaaatcaggcactgcatctgtggtctgcctgctgaacaacttctaccccagggaggccaaggtccagtggaaggtggacaatgccctgcagagtgggaatagccaggagtctgttacagagcaggactccaaggacagcacatacagcctctcctctactttgaccttgtccaaagcagattatgagaagcacaaagtatatgcctgtgaggtgacacatcaagggctcagcagccctgtgaccaaatccttcaacagaggagaatgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 150 - DNA, 3852 bp, synthetic construct
  source 1..3852: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcacttccctaatgagagggataagctcttctgggtgtagtggttgtggagggcttcatgcatcacactgcaggagaagacatttccttcctgccacctggatttatccacagtcaacctgctgtacaggaagaagcttccatcactgtccagcactggtggtgtggttttgtagttgttttcaggctgcccattactctcccattcaactgcaatatctgaaggataaaagcctttcacaagacaagttagagacacttgatttttggtcatctcctcctgggaaggtggcagtgtgtagacctggggctctctgggctggccctttgctttggagatggtcttttcaatggaggagggtaggcccttgttggacactttgcacttgtactccttgccattcagccagtcctggtgcaagacagtgagcacactgactactctataggtgctgttgaactgctcttctctaggctttgtcttggcattatgcacctccaccccatccacataccaattaaactgaacttctggatcttcttgagacacatcaaccacaacacaggtgacctctggggttcttgaaatcatcagagtatccttgggtttgggggggaagagaaatacactgggcccagcaactgggggggcagggcagggtggacattccacacagcatttcctttctactgttttgtccaccttggtgttgctgggcttgtggtccacattgcaggtatatgtctgagtgccaaagttagagcttggaactgtcactactgaactaagtgaataaagcccagagctctgcaggacagctggaaaagtatgaacaccagaggtgagagcacctgaattccagcttacagtcacaggctcagggaagtagtccttcaccaggcagcccagggctgcagtactttcagaagtggacctggaacatggagccaaaggaaaaacagaaggtcctttagtgctagctgatgagacagttaccaatgtgccttgtccccagacatcaaagtaccagttgggactagatccaaaaaaatatctggcacaataatacacagctgtgtcctcagatctaagggagctcagctccatgtacactgtggaggtgctggtgtctcttgtcatggttaccctgtctttgaagttctctgtatactctgtgctgccactcccaggtaatatttctcccatccactccaggccttggccaggggcttgcctgacccactggatccagtaattactgaagatgtagccagatgccttgcagctaactttgactgaggcacctggcttcttgacttctgctccagactggaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacccaaagccccagctccttgtcagcttcagttggtgacagggtaacaattacttgtggggcttcagaaaatatttatggagcactcaactggtaccagcagaagccaggcaaggccccaaagttgttaatatatggggccacaaacctggcagatggggtaccctccaggttttctggttcaggcagtgggacagacttcaccctcaccatcagcagtcttcaaccagaagattttgccacctactactgccagaatgtgctgaatactcctcttacatttggacaaggcaccaaggtggagattaaaagaactgtggctgcacccagtgtcttcatctttcctccttctgatgaacagctgaagagtggcacagcatctgtggtgtgcctgctgaacaacttctacccaagagaggccaaggtccagtggaaagtggacaatgccctgcaaagtggaaattcccaggagtctgttacagagcaggacagcaaagattctacttacagcctaagcagcactttaactctttccaaggctgattatgaaaaacataaagtttatgcctgtgaggtgacccaccagggcctctcctctcctgtcaccaaatccttcaacagaggagaatgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 151 - DNA, 3852 bp, synthetic construct
  source 1..3852: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcattttcctaaactgagggagagactcttctgggtgtagtggttgtgcagagcttcatgcatgacagaacatgaaaagacattcccttcctgccatctggacttgtctactgtcagcctgctatacaggaagaaggaaccatctgagtctagtactggaggggtggtcttataattattttctggctgcccattactctcccattccacagcaatatctgaggggtagaagcccttcacaaggcaggtcaaggacacctggttttttgtcatttcttcttgtgatggggggagagtatagacttgtggctccctgggctggcctttggctttgctgatagtcttctctatgctggagggcaagcctttgttactgactttgcatttatactcctttccattcagccagtcctgatggaggacagtgagcacagaaacaactctataagtgctgttaaactgctcctctctaggcttggttttagcattatgaacttctactccatcaacataccaattaaattgcacctcagggtcctcctgggaaacatcaaccaccacacaggtgacttctggagtcctactaatcatgagtgtgtccttgggcttaggggggaatagaaagactgaagggcctgcaactgggggggcagggcatgggggacactcaacacagcattttctctccacagttttgtccacttttgtattgctgggcttgtggtccacattgcaagtgtaggtctgggttccaaagttgctagatggtactgtgaccacagatgacagggaataaaggccactgctctgcagtactgcagggaaggtgtgcaccccacttgtcaaagcaccactattccatgaaacagtcacaggctcaggaaaatagtctttcaccaggcagcccagggctgctgtgctctcagatgtggacctactgcagggggctaatgggaagacagatggtcctttagttgaggcagaagacactgtcaccaaggtgccctggccccagacatcaaaataccagttggggctgctgccaaagaagtatctggcacagtagtagactgctgtatcttctgacctaaggctggacagctccatgtacactgtagaggtggaggtgtctctggtcatggtcactctatccttgaagttttctgtatattcagtggagccagacccaggaagaatctcccccatccactccaggccttgcccaggggcttgtctgacccattgaatccagtagttggaaaagatgtaaccagatgccttgcagctgactttgactgaggcccctggcttcttcacttcagctccactctggaccagctgtacttggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacccagagtcccagtagtctatcagcctctgtaggggatagagttaccatcacctgtggggcttcagaaaatatatatggggccctcaactggtaccagcagaaaccaggaaaagcacccaagctcttgatttatggagccacaaatttggcagatggagtacccagcaggttttctggttctggctcaggcactgacttcaccttaactatttcctccctgcaacctgaggactttgcaacttactactgccagaatgtgctgaatacacctcttacctttggccaagggacaaaagtggagatcaagaggactgtggctgcaccatctgtcttcatttttcccccttctgatgaacagctgaagagtggtacagcttctgtggtgtgcctgctgaacaacttctacccaagagaagccaaggttcagtggaaggtggacaatgccctgcaaagtggcaacagccaggagtcagtcacagagcaggacagcaaagattccacctacagcctctccagcactcttacactttccaaagctgattatgagaagcacaaagtttatgcctgtgaggtgactcatcaaggcctgagcagccctgtcaccaagtccttcaacagaggagaatgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 152 - DNA, 3731 bp, synthetic construct
  source 1..3731: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 153 - DNA, 3853 bp, synthetic construct
  source 1..3853: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 154 - DNA, 3777 bp, synthetic construct
  source 1..3777: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 155 - DNA, 3777 bp, synthetic construct
  source 1..3777: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagctgctggaggatgggaactgaggggttggaagggggcagggtgagcccagaaactcctgtgtgcctcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 156 - DNA, 4022 bp, synthetic construct
  source 1..4022: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaaagggggaggctgctggtgaatattaaccaaggtcaccccagttatcggaggagcaaacaggggctaagtccactggctgggatctgagtcccccacctactctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 157 - DNA, 4022 bp, synthetic construct
  source 1..4022: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcttgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 158 - DNA, 3816 bp, synthetic construct
  source 1..3816: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagctccagcaagaggtaagggtttaagggatggttggttggtggggtattaatgtttaattacctggagcacctgcctgaaatcactttttttcaggttggactggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 159 - DNA, 4058 bp, synthetic construct
  source 1..4058: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 160 - DNA, 4022 bp, synthetic construct
  source 1..4022: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 161 - DNA, 3731 bp, synthetic construct
  source 1..3731: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggactggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 162 - DNA, 4634 bp, synthetic construct
  source 1..4634: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccccttgctccataccacccccctccaccccagagacacaaaaaattccaacacacgagacaaaattgttgttaacttgtttattaggacaaggctggtcggcactggagtggcaacttccagggccaggagaggtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 163 - DNA, 4548 bp, synthetic construct
  source 1..4548: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcgaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 164 - DNA, 4595 bp, synthetic construct
  source 1..4595: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgaggaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatcacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 165 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 166 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 167 - protein, 19 aa, synthetic construct
MGWSCIILFLVATATGVHS

SEQ ID NO: 168 - DNA, 57 bp, synthetic construct
  source 1..57: mol_type = other DNA
atgggctggtcctgcatcatcctgttcctggtggccaccgccacaggcgtgcacagc

SEQ ID NO: 169 - DNA, 57 bp, synthetic construct
  source 1..57: mol_type = other DNA
atgggctggagctgcatcatcctcttcctggtggccacggccacaggtgtccacagc

SEQ ID NO: 170 - DNA, 57 bp, synthetic construct
  source 1..57: mol_type = other DNA
atgggctggagctgcatcatcctcttcctggtggccacagccacaggtgtccacagc

SEQ ID NO: 171 - DNA, 57 bp, synthetic construct
  source 1..57: mol_type = other DNA
atgggctggtcatgtatcatcctgttcctggtagccactgccacaggggttcatagc

SEQ ID NO: 172 - DNA, 57 bp, synthetic construct; identical to SEQ ID NO: 170
  source 1..57: mol_type = other DNA

SEQ ID NO: 173 - protein, 22 aa, synthetic construct
MDMRVPAQLLGLLLLWLSGARC

SEQ ID NO: 174 - DNA, 66 bp, synthetic construct
  source 1..66: mol_type = other DNA
atggacatgagggtccctgctcagctgctggggctcctgctgctctggctcagcggtgccagatgt

SEQ ID NO: 175 - DNA, 66 bp, synthetic construct
  source 1..66: mol_type = other DNA
atggacatgagagttcctgcacagctgctggggctcctgctgctctggctctctggtgcccgctgt

SEQ ID NO: 176 - DNA, 66 bp, synthetic construct
  source 1..66: mol_type = other DNA
atggacatgagagtccctgcacagctgctggggctcctgctgctctggctctctggggcccgctgt

SEQ ID NO: 177 - DNA, 66 bp, synthetic construct
  source 1..66: mol_type = other DNA
atggacatgagggtgccagcacagctgctgggcctcctgctgctgtggctgagtggtgcaagatgt

SEQ ID NO: 178 - protein, 448 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 179 - protein, 214 aa, synthetic construct [sequence omitted]

SEQ ID NO: 180 - DNA, 1344 bp, synthetic construct
  source 1..1344: mol_type = other DNA
caagtgcagctggttcaaagcggagccgaagtgaaaaagcccggagctagcgtgaaggtgtcctgcaaggccagcggccacatcttcagcaactactggatccagtgggtgcggcaggcccctggccaaggcctggaatggatgggcgagatcctgccaggatctggccacaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgctccacgaagccctgcacagccactacacccagaagtccctgagcctgtctctgggaaaa

SEQ ID NO: 181 - DNA, 1344 bp, synthetic construct
  source 1..1344: mol_type = other DNA
caagtgcagctggttcaaagcggagccgaagtgaaaaagcccggagctagcgtgaaggtgtcctgcaaggccagcggctatatcttcagcaactactggatccagtgggtgcggcaggcccctggccaaggcctggaatggatgggcgagatcctgccaggatctggctctaccgagtacacagagaacttcaaggatagagtgaccatgaccagagatacctccacaagcaccgtgtacatggaactgagcagcctgagaagcgaggacacagctgtgtactactgcgccagatacttttttggctcatcccctaactggtacttcgacgtgtggggccaaggcacccttgtcaccgtcagcagcgccagtactaagggacccagcgtgttcccactggccccatgcagcagaagcacatctgaaagcacagccgccctgggttgtctggtcaaagactacttccccgaacccgtgacagtgagctggaacagcggcgccctgacaagcggcgtgcacaccttcccagccgtgctgcagagctctggcctgtattctctgagtagcgtggtcaccgttcctagctccaacttcggcacacagacctacacctgtaatgtggaccacaagcctagcaacaccaaagtggataagaccgttgagagaaagtgctgcgtggaatgcccaccatgtccagctccaccagtcgccggcccttctgttttcctgttcccaccaaagcccaaagacaccctgatgatcagccggacccctgaagtgacatgtgtggtggtggatgtgtcccaggaggatcctgaggtgcagtttaattggtacgttgacggagtggaagttcataatgccaagaccaaaccccgcgaggaacagtttaacagcacctaccgggtggtgtccgtgctgacagtgctgcaccaggactggctgaacggcaaagagtacaagtgcaaggtgtctaacaagggcctgcctagcagcatcgagaagaccatctcaaaggccaagggccagcccagagagccccaagtctatacactgcctccttctcaagaagaaatgacaaagaaccaagtgtctctgacctgcctggtgaagggcttctaccccagcgacatcgccgtcgaatgggagagcaacggacagcctgaaaacaactacaagacgacccctccagtgctggacagcgatggcagcttcttcctgtattcacggctgaccgtggacaagagccgatggcaagagggcaacgtgtttagctgcagcgtgatgcacgaagccctgcacaaccactacacccagaagtccctgagcctgtctctgggaaaa

SEQ ID NO: 182 - DNA, 1344 bp, synthetic construct
  source 1..1344: mol_type = other DNA
caagtacagcttgtacaaagtggcgctgaagtaaaaaagccaggggcttcagtgaaagtgtcctgcaaggcctcgggctacatcttctccaactactggatccagtgggtgcgccaggccccgggccaagggctggagtggatgggagagattttacctggcagtggctccacagagtacacagagaacttcaaggacagagtcaccatgacccgggacaccagcacatcaactgtctacatggagctgagctccctccgatcagaagacacagctgtctactactgtgcccgctacttctttggcagctcccccaactggtactttgatgtgtggggccaaggcaccttggtcaccgtctcctcagcatcaacaaaaggtccttctgtcttccccctggccccctgcagccgcagcacatcagaatccacagcagccctgggctgcctggtaaaagactacttcccagaacctgtcactgtctcctggaacagtggagccctgacatctggtgtccacaccttccctgctgtcctccagagctctggcctctacagcctctcctcagtggtcacagttccatcctccaactttggcacacagacctacacctgcaatgtggaccacaagccttccaacaccaaggtggacaagactgtagagaggaagtgctgtgtggaatgcccaccctgcccagctccacctgttgctgggccttctgtcttcctcttccctccaaagcccaaagacaccctcatgatcagccgcaccccagaagtgacctgtgtggtggtggatgtctcccaggaagaccctgaggtgcagttcaactggtatgtagatggtgttgaagttcataatgccaagaccaagccccgggaggagcagttcaacagcacctacagagtggtgtctgtgctgacagtgctgcaccaggactggctgaatggaaaagaatacaagtgcaaagtttccaacaagggcctgccttcctccattgagaagaccatctccaaagccaagggacagccccgggagccccaagtctacacacttcctccttctcaagaagaaatgacaaagaaccaagtgtctctgacctgcctggtgaagggcttctacccaagtgacattgctgtagaatgggagagcaatggacagcctgaaaacaactacaaaacgaccccacctgtgctggactctgatggcagcttcttcttgtattcacgcctgactgtggacaagagccgctggcaagaaggaaatgtattttcctgctctgtgatgcatgaggccctgcacaatcactacacccagaagtcactcagcctctctctgggaaaa

SEQ ID NO: 183 - DNA, 1344 bp, synthetic construct
  source 1..1344: mol_type = other DNA
caagtgcagctggttcaaagtggggctgaagtaaaaaagcctggggcttctgtgaaggtgtcctgcaaggcctcgggctacatcttctccaactactggatccagtgggtccggcaggcccctgggcaaggcctggagtggatgggagagattcttcctggaagtggatccacagagtacacagaaaacttcaaggacagagtcaccatgaccagggacacctccacatccacggtgtacatggaactttccagcctgagatctgaggacactgctgtctactactgtgcccgctacttctttggcagcagccccaattggtattttgatgtctggggccaagggaccttggtcactgtctcctcagcatccacaaaaggaccctcagtcttccctctggccccctgctcccgctccacatcagaaagcacagctgccctgggttgtcttgtaaaagattattttccagaacctgtcactgtgtcctggaacagtggggccctgacttcaggagtccacaccttcccagctgtgctgcagagcagtggcctctattctttatcatctgtggtcactgttccttccagcaactttggcacacagacctacacctgtaatgtggaccacaaaccttccaacacaaaagtggacaaaactgttgaaagaaaatgctgtgtggaatgtccaccctgccctgctccaccagttgctgggcccagtgtcttcctcttccctcccaagcccaaggacaccctcatgatcagccgcaccccagaagtgacctgtgtggtggtggatgtttctcaagaagatcctgaggtgcagtttaattggtatgtcgatggtgttgaagttcataatgccaagacaaaaccccgggaggagcagtttaacagcacctacagagttgtttctgtcctcactgtgctgcaccaggactggctgaatggaaaagaatataaatgcaaagtgagcaacaaaggcctgcccagcagcattgagaagaccatctcaaaggccaagggccagccccgggagccccaagtatatactcttccacccagccaagaagaaatgacaaagaaccaagtatctctcacctgcctggtgaaaggattttatccttctgatattgctgtagaatgggagagtaatggacagccagaaaacaactacaagacgacgccgccggtgctggacagtgatggcagcttcttcctctattcacgcctcactgtggacaagagccgctggcaagaaggaaatgtcttctcctgctctgtcatgcatgaagccctgcacaaccactacacacagaagtccctgagcctctccctggggaag

SEQ ID NO: 184 - DNA, 1344 bp, synthetic construct
  source 1..1344: mol_type = other DNA
caagtgcaattggtccagtcaggtgcagaggttaagaagcctggggcatctgttaaggtttcctgcaaggcatcaggttacattttcagcaactactggattcaatgggtgaggcaggcaccaggtcaaggattggaatggatgggggaaatactgcctgggtcaggatccacagagtacacagagaacttcaaagatagggtcaccatgactagagacacatctactagcactgtttacatggagctcagtagccttaggtcagaagacactgctgtctactactgtgccagatatttctttggcagcagccctaattggtactttgatgtatggggccagggcaccctggtgactgtgagcagtgcttccacaaagggcccatcagtcttcccattggcaccttgtagcaggagcacttcagagagcacagctgcactgggttgcttggtgaaggactacttcccagaaccagtgacagtgtcctggaacagtggtgcacttacatcaggagtgcacaccttccctgcagtactccaatcaagtggcctttacagcctctccagcgttgtcacagtcccctcatctaactttggaactcagacctatacctgtaatgtggaccacaagccttccaacaccaaagtggacaagacagtagaaaggaaatgctgtgtggagtgcccaccgtgcccagccccaccagttgcaggcccaagtgtgttcctcttcccccccaagcccaaagacaccctgatgatcagtaggacccctgaggtgacctgtgttgtggtggatgtgagccaggaggatcctgaggtgcagtttaattggtacgttgacggagtggaagttcataatgccaaaactaagcctagggaggagcagttcaatagcacctacagggtggtgtctgttcttacagtcctgcaccaagactggctgaatggcaaagaatacaagtgcaaagtcagcaacaaggggctgcctagctctattgagaagaccatcagcaaagccaaaggacagcctagagaaccccaggtgtataccttgcctccctcccaagaagagatgaccaagaaccaagtgagcctgacttgccttgtgaagggcttctacccttcagatatagctgttgagtgggagagcaatggccagccagaaaacaactacaaaaccaccccacctgtattggatagtgatggaagctttttcttgtacagcaggctgactgttgataagagcaggtggcaggagggcaatgtgttcagttgttctgtgatgcatgaggccctgcacaatcactacacccagaagagtctgtccctttctctgggcaag

SEQ ID NO: 185 - DNA, 1344 bp, synthetic construct
  source 1..1344: mol_type = other DNA
caggtgcagctggtgcagtctggggccgaggtgaagaaaccaggcgcctccgtgaaggtgtcttgcaaagccagcgggtacatcttctccaactattggattcagtgggtcaggcaggcaccaggacagggactggagtggatgggcgaaatcctgcccgggtctggcagcacagagtacactgaaaattttaaggacagagtgactatgaccagggatacatccacttctaccgtgtatatggagctgagctccctgagaagcgaagacactgccgtgtactattgtgccaggtacttctttggctctagccctaactggtatttcgatgtgtggggacaggggaccctggtgacagtgtcctctgcctccacaaagggcccatctgtgtttcccctggccccttgcagcaggtccacttctgagagcaccgcagcactgggatgtctggtgaaagactacttccctgaaccagtgaccgtgagctggaactccggagcactgaccagcggagtgcacacatttccagcagtgctgcagagctccggactgtactccctgtctagcgtggtgacagtgccctcctctaatttcggcacacagacttatacctgcaacgtggaccataagcctagcaatactaaggtggataaaaccgtggagcggaaatgctgtgtggaatgcccaccttgtcctgcaccaccagtggcaggaccaagcgtgttcctgtttcctccaaagcccaaagacaccctgatgatctctagaacacctgaagtgacttgtgtggtggtggacgtgagccaggaagacccagaagtgcagttcaactggtacgtggatggggtggaagtgcacaatgccaagacaaaacccagggaggaacagtttaatagcacctaccgggtggtgtccgtgctgacagtgctgcatcaggattggctgaacggaaaggagtataagtgcaaagtgtccaataaggggctgcctagctccatcgagaaaactatttctaaggccaaaggccagccaagagaaccccaggtgtacaccctgccccctagccaggaagagatgacaaagaaccaggtgtccctgacttgtctggtgaaaggcttctatccttccgacattgccgtggagtgggaatctaatggacagccagaaaacaattacaagaccacaccacccgtgctggactctgatggaagcttctttctgtattctcggctgacagtggataaaagcagatggcaggaaggcaacgtgttttcctgctctgtgatgcacgaagccctgcacaatcattacactcagaagagcctgtccctgtctctgggaaaa

SEQ ID NO: 186 - DNA, 1344 bp, synthetic construct
  source 1..1344: mol_type = other DNA
caggtgcagctggtgcagtctggagcagaggtgaagaaaccaggagcctccgtgaaggtgtcttgcaaagccagcggctacatcttctccaactattggattcagtgggtcaggcaggcaccaggacagggactggagtggatgggagagatcctgccaggatctggaagcaccgagtacacagagaattttaaggacagagtgaccatgacaagggatacctccacatctaccgtgtatatggagctgagctccctgaggagcgaggacaccgccgtgtactattgtgcccgctacttctttggctctagccctaactggtatttcgatgtgtggggccagggcaccctggtgacagtgtcctctgcctccaccaagggcccatctgtgtttccactggcaccttgcagcaggtccacctctgagagcacagcagcactgggctgtctggtgaaagactacttccctgagccagtgaccgtgagctggaactccggagcactgaccagcggagtgcacacatttccagcagtgctgcagagctccggactgtactccctgtctagcgtggtgacagtgccctcctctaatttcggcacacagacctatacatgcaacgtggaccacaagcctagcaataccaaggtggataaaacagtggagagaaaatgctgtgtggagtgcccaccttgtcctgcaccaccagtggcaggcccatccgtgttcctgtttcctccaaagcccaaagacaccctgatgatctctaggacccctgaggtgacatgtgtggtggtggacgtgagccaggaagacccagaggtgcagttcaactggtacgtggatggcgtggaggtgcacaatgccaagaccaaacccagagaggagcagtttaatagcacctacagggtggtgtccgtgctgacagtgctgcaccaggattggctgaacggcaaggagtataagtgcaaagtgtccaataagggcctgcctagctccatcgagaaaaccatttctaaggccaaaggccagccaagagagccccaggtgtacacactgccccctagccaggaagagatgaccaagaaccaggtgtccctgacatgtctggtgaaaggcttctatccttccgacattgccgtggagtgggagtctaatggccagccagagaacaattacaagaccacaccacccgtgctggactctgatggcagcttctttctgtattctcggctgaccgtggataaaagccgctggcaggaaggcaacgtgttttcctgctctgtgatgcacgaggccctgcacaatcactacacacagaagagcctgtccctgtctctgggcaaa

SEQ ID NO: 187 - DNA, 1344 bp, synthetic construct
  source 1..1344: mol_type = other DNA
caggtccagctggtgcagagcggagccgaagtcaagaagcccggagcctcagtcaaagtgtcatgtaaagccagtgggtatattttcagcaactactggatccagtgggtcaggcaggctcctggacagggcctggagtggatgggagagatcctgcctggaagcggatccaccgagtacaccgagaatttcaaggacagagtgaccatgacccgcgataccagcacctccaccgtgtacatggagctgagctccctgaggagcgaggacaccgccgtgtactactgcgccaggtacttctttggcagctcccccaactggtacttcgatgtgtggggacagggaaccctggtgaccgtgagctccgctagcaccaagggaccttccgtgtttcctctggctccttgcagcaggtccaccagcgagtccaccgctgctctgggatgtctggtgaaggactacttccccgagcctgtgaccgtgagctggaactccggagctctgaccagcggagtgcacacctttcctgctgtgctgcagagctccggcctgtactccctgagctccgtggtgaccgtgcccagctccaatttcggcacccagacctacacctgcaacgtggaccacaagcctagcaataccaaggtggataagaccgtggagagaaagtgctgtgtggagtgccctccttgtcctgctcctcctgtggctggcccttccgtgttcctgtttccccctaagcccaaggacaccctgatgatcagccgcacccctgaggtgacctgtgtggtggtggacgtgtcccaggaggatcccgaggtgcagttcaactggtacgtggatggcgtggaggtgcacaatgccaagaccaagcctagagaggagcagtttaacagcacctaccgcgtggtgtccgtgctgaccgtgctgcaccaggattggctgaacggcaaggagtacaagtgcaaggtgagcaataagggcctgcccagctccatcgagaagaccatctccaaggccaagggccagcccagagagcctcaggtgtacaccctgccccctagccaggaggagatgaccaagaaccaggtgtccctgacctgtctggtgaagggcttctaccccagcgacatcgccgtggagtgggagtccaatggccagcctgagaacaattacaagaccaccccccctgtgctggacagcgatggctccttctttctgtacagccggctgaccgtggataagtccaggtggcaggagggcaatgtgtttagctgttccgtgatgcacgaggctctgcacaatcattacactcagaaatccctgtcactgtccctgggcaag

SEQ ID NO: 188 - DNA, 1344 bp, synthetic construct
  source 1..1344: mol_type = other DNA
caggtgcagctggtgcagagcggagcagaggtgaagaagcctggagcctccgtgaaagtgagctgcaaggcctccggctacatcttctccaactactggatccagtgggtcagacaggcacctggacagggactggagtggatgggagagatcctgcctggaagcggatccaccgagtacaccgagaattttaaggacagagtgaccatgaccagggataccagcacctccaccgtgtacatggagctgagctccctgagaagcgaggacaccgccgtgtactactgtgccaggtacttctttggcagctcccccaactggtacttcgatgtgtggggacagggaaccctggtgaccgtgagctccgcaagcaccaagggaccttccgtgtttcctctggcaccttgcagcaggtccaccagcgagtccaccgcagcactgggatgtctggtgaaggactacttccccgagcctgtgaccgtgtcctggaacagcggagcactgaccagcggagtgcacacctttcctgcagtgctgcagagctccggactgtactccctgagctccgtggtgaccgtgcccagctccaatttcggcacccagacctacacctgcaacgtggaccacaagcctagcaataccaaggtggataagaccgtggagagaaagtgctgtgtggagtgccctccttgtcctgcacctcctgtggcaggcccttccgtgttcctgtttccccctaagcccaaggacaccctgatgatcagcaggacccctgaggtgacctgtgtggtggtggacgtgtcccaggaagaccccgaggtgcagttcaactggtacgtggatggcgtggaggtgcacaatgccaagaccaagcctagagaggagcagtttaatagcacctacagggtggtgtccgtgctgaccgtgctgcaccaggattggctgaacggcaaggagtacaagtgcaaagtgagcaataagggcctgcctagctccatcgagaagaccatctccaaggccaagggccagcccagagagcctcaggtgtacaccctgccccctagccaggaagagatgaccaagaaccaggtgtccctgacctgtctggtgaagggcttctaccccagcgacatcgccgtggagtgggagtccaatggccagcctgagaacaattacaagaccaccccccctgtgctggactccgatggcagcttctttctgtacagcagactgaccgtggataagtccaggtggcaggaaggcaacgtgtttagctgctccgtgatgcacgaggccctgcacaatcactacacccagaagagcctgtccctgagcctgggcaag

SEQ ID NO: 189 - DNA, 1344 bp, synthetic construct
  source 1..1344: mol_type = other DNA
caggtgcagctggtgcagtctggggccgaggtgaagaaacctggcgcctccgtgaaggtgtcttgcaaagccagcgggtacatcttcagcaattattggattcagtgggtcaggcaggcaccaggacagggactggagtggatgggcgaaatcctgcctgggtctggcagcacagagtacactgaaaactttaaggacagagtgactatgaccagggatacatccacttctaccgtgtatatggagctgagctccctgagaagcgaagacactgccgtgtactattgtgccaggtacttctttggctctagccccaattggtatttcgatgtgtggggacaggggaccctggtgacagtgtcctctgcctccacaaagggcccatctgtgtttcctctggccccctgcagcaggtccacttctgagagcaccgcagcactgggatgtctggtgaaagactacttccccgaaccagtgaccgtgagctggaattccggcgccctgaccagcggagtgcacacatttcctgccgtgctgcagagctccgggctgtactccctgtctagcgtggtgacagtgccttcctctaacttcggcacacagacttatacctgcaatgtggaccataagccctccaacactaaggtggataaaaccgtggagcggaaatgctgtgtggaatgccctccatgtccagcaccacctgtggcaggaccaagcgtgttcctgtttcccccaaagcctaaagacaccctgatgatctctagaacacccgaagtgacttgtgtggtggtggacgtgagccaggaagacccagaagtgcagttcaattggtacgtggatggggtggaagtgcacaacgccaagacaaaacctagggaggaacagtttaacagcacctaccgggtggtgtccgtgctgacagtgctgcatcaggattggctgaatggaaaagagtataagtgcaaagtgtccaacaaggggctgcccagctccatcgagaaaactatttctaaggccaaaggccagccaagagaacctcaggtgtacaccctgcctcccagccaggaagagatgacaaagaatcaggtgtccctgacttgtctggtgaaaggcttctatccctccgacattgccgtggagtgggaatctaacggacagccagaaaataactacaagaccacaccacctgtgctggactctgatggaagcttctttctgtattctcggctgacagtggataaaagcagatggcaggaaggcaatgtgttttcctgctctgtgatgcacgaagccctgcacaaccattacactcagaagagcctgtccctgtctctgggaaaa

SEQ ID NO: 190 - DNA, 1344 bp, synthetic construct
  source 1..1344: mol_type = other DNA
caggtgcagctggtgcagtctggagctgaggtgaagaagcctggagccagtgtgaaggtgagctgcaaggcctctggctacatcttcagcaactactggatccagtgggtcagacaggcccctggtcaaggcctggagtggatgggagaaatcctgcctggctctggcagcacagagtacacagagaacttcaaggacagagtgacaatgaccagagataccagcacctccacagtgtacatggaactgtccagcctgagatctgaggacacagctgtgtactactgtgccagatacttctttggcagcagccctaactggtactttgatgtgtggggccagggcaccctggtgacagtgagctctgccagcaccaagggcccttctgtcttccctctggccccttgcagcagaagcacatctgagtcaacagctgccctgggctgcctggtgaaggattacttccctgaacctgtgactgttagctggaactctggagccctgacctctggagtacacaccttccctgctgtcctgcagagcagtggcctgtactccctgagctctgtggtgacagtgccaagcagcaactttggcacacagacctacacctgcaatgtggaccacaagcccagcaacaccaaggtggacaagacagtggagaggaagtgctgtgtggaatgccccccctgccctgcccctcctgtggctggcccctctgtgttcctgttcccccccaaacccaaggacaccctgatgatctccagaaccccagaagtgacctgtgtggtggttgatgtgagccaggaagaccctgaggtccagtttaactggtatgtggatggagtggaggttcacaatgccaagaccaagccaagggaggagcagttcaacagcacctacagagtggtgtcagtgctaactgttctgcaccaggactggctcaatggcaaggagtacaaatgtaaggtgagcaacaagggcctccccagcagcatagaaaaaaccatcagcaaggccaagggccaacccagagagcctcaggtgtataccctgcctcctagccaagaggaaatgacaaaaaaccaggtgagcctgacctgcctggtgaagggcttctacccctcagacattgcagtggaatgggaaagcaatggccagcctgagaacaactataagaccaccccacctgtgctggactctgatggcagcttcttcctgtacagcaggctgacagtggacaagagcagatggcaagagggcaatgttttcagctgctctgtgatgcatgaggccctgcacaaccactacacccagaaaagcctgagcctgtctctgggcaaa

SEQ ID NO: 191 - DNA, 1344 bp, synthetic construct
  source 1..1344: mol_type = other DNA
caggtgcagctggtgcagtctggagctgaggtgaagaagcctggagccagtgtgaaggtgtcctgcaaggcctctggctacatcttcagcaactactggatccagtgggtcagacaggcccctggtcaaggcctggagtggatgggagaaatcctgcctggctctggcagcacagagtacacagagaacttcaaggacagagtgacaatgaccagagataccagcacctccacagtgtacatggaactgtccagcctgagatctgaggacacagctgtgtactactgtgccagatacttctttggcagcagccctaactggtactttgatgtgtggggccagggcaccctggtgacagtgagctctgccagcaccaagggcccttctgtcttccctctggccccttgcagcagaagcacatctgagtcaacagctgccctgggctgcctggtgaaggattacttccctgaacctgtgactgttagctggaactctggagccctgacctctggagtacacaccttccctgctgtcctgcaaagcagtggcctgtactccctgagctctgtggtgacagtgccaagcagcaactttggcacacagacctacacctgcaatgtggaccacaagcccagcaacaccaaggtggacaagacagtggagaggaagtgctgtgtggaatgccccccctgccctgcccctcctgtggctggcccctctgtgttcctgttcccccccaaacccaaggacaccctgatgatctccagaaccccagaagtgacctgtgtggtggttgatgtgagccaggaagaccctgaggtccagtttaactggtatgtggatggagtggaggttcacaatgccaagaccaagccaagggaggagcagttcaacagcacctacagagtggtgtcagtgctaactgttctgcaccaggactggctcaatggcaaggagtacaaatgtaaagtgtccaacaagggcctccccagcagcatagaaaaaaccatcagcaaggccaagggccaacccagagagcctcaggtgtataccctgcctcctagccaagaggaaatgacaaaaaaccaggtgagcctgacctgcctggtgaagggcttctacccctcagacattgcagtggaatgggaaagcaatggccagcctgagaacaactataagaccaccccacctgtgctggactctgatggcagcttcttcctgtacagcaggctgacagtggacaagagcagatggcaagagggcaatgttttcagctgctctgtgatgcatgaggccctgcacaaccactacacccagaaaagcctgagcctgtctctgggcaaa

SEQ ID NO: 192 - DNA, 1344 bp, synthetic construct
  source 1..1344: mol_type = other DNA
caagtccagctggtgcagagtggggctgaagtaaagaagccaggtgcctcagtgaaagtcagctgcaaggcatctggctacatcttcagcaactactggatccagtgggtgaggcaggcccctggccagggcctggagtggatgggggagatcctcccaggttcaggcagcacagagtatactgagaacttcaaggacagagtgaccatgactagggacaccagtaccagcacagtctacatggagctgagttccttgaggtctgaagacacagcagtctactactgtgccagatatttctttggatccagcccaaattggtactttgatgtgtggggacaaggtactttggtgacagtttcctctgcctcaacaaaaggcccctctgtgtttcccctggcaccctgcagcagatcaacttcagaatccactgcagccctggggtgccttgtcaaagactacttccctgagcctgtgactgtatcctggaattctggggccctgacctctggagtgcatacctttccagctgttctgcagagctcaggtctttattccttgagttcagttgtaacagtcccctcatccaattttgggacccagacttacacctgcaatgtggaccacaagccttctaacaccaaggtggacaagacagttgaaagaaagtgctgtgtggagtgtcctccttgcccagccccccctgttgctgggcccagtgtgttcctcttccccccaaagcccaaggatacactaatgatctccaggacaccagaagtcacctgtgttgtggtggatgtttcccaggaagatccagaggtgcagtttaactggtatgttgatggagtggaggtccataatgccaagactaaacccagggaggagcagttcaacagcacctatagggtggtaagtgtcctcacagtgctccaccaggactggctgaatggcaaagagtacaagtgtaaagtgagcaacaagggactccccagctccattgagaagaccataagcaaagctaaaggtcaacctagagaacctcaggtgtataccctgcccccttctcaagaagagatgaccaagaaccaggtttctcttacttgtcttgtgaagggcttctaccctagtgacattgctgtggaatgggagagcaatgggcagccagaaaacaactataaaaccacccccccagtgctggactctgatggcagcttcttcctgtattccaggttgactgtagataaaagtagatggcaggagggcaatgtcttttcctgctctgtcatgcatgaggctctgcacaaccactacacacagaagagcctgtcattatccctaggaaaa

SEQ ID NO: 193 - DNA, 1344 bp, synthetic construct
  source 1..1344: mol_type = other DNA
caagtccagctggtgcagagtggggctgaagtaaagaagccaggtgcctcagtgaaagtcagctgcaaggcatctggctacatcttcagcaactactggatccagtgggtcaggcaggcccctggccagggcctggagtggatgggggagatcctcccaggttcaggcagcacagagtatactgagaacttcaaggacagagtgaccatgactagggacaccagtaccagcacagtctacatggagctgagttccttgaggtctgaagacacagcagtctactactgtgccagatatttctttggatccagcccaaattggtactttgatgtgtggggacaaggcactttggtgacagtttcctctgcctcaacaaaaggcccctctgtgtttcccctggcaccctgcagcagatcaacttcagaatccactgcagccctggggtgccttgtcaaagactacttccctgagcctgtgactgtatcctggaattctggggccctgacctctggagtgcatacctttccagctgttctgcagagctcaggtctttattccttgagttcagttgtaacagtcccctcatccaattttgggacccagacttacacctgcaatgtggaccacaagccttctaacaccaaggtggacaagacagttgaaagaaagtgctgtgtggagtgtcctccttgcccagccccccctgttgctgggcccagtgtgttcctcttccccccaaagcccaaggatacactaatgatctccaggacaccagaagtcacctgtgttgtggtggatgtttcccaggaagatccagaggtgcagtttaattggtatgtggatggagtggaggtccataatgccaagactaaacccagggaggagcagttcaacagcacctatagggtggtaagtgtcctcacagtgctccaccaggactggctgaatggcaaagagtacaagtgtaaagtgagcaacaagggactccccagctccattgagaagaccataagcaaagctaaaggtcaacctagagaacctcaggtgtataccctgcccccttctcaagaagagatgaccaagaaccaggtttctcttacttgtcttgtgaagggcttctaccctagtgacattgctgtggaatgggagagcaatgggcagccagaaaacaactataaaaccacccccccagtgctggactctgatggcagcttcttcctgtattccaggttgactgtagataaaagtagatggcaggagggcaatgtcttttcctgctctgtcatgcatgaggctctgcacaaccactacacacagaagagcctgtcattatccctaggaaaa

SEQ ID NO: 194 - DNA, 1344 bp, synthetic construct
  source 1..1344: mol_type = other DNA
caagtgcagctggtgcagagtggagctgaggtgaagaagccaggtgccagtgtaaaagtgagctgcaaagcatctggctacattttcagtaactactggatccagtgggtgaggcaagccccagggcaaggattggaatggatgggggagatcttgccaggttctggaagcacagaatatacagagaactttaaggatagagtgaccatgaccagagacaccagcacttccacagtctacatggagctgtcctctctcagatctgaagatacagcagtctactactgtgccaggtatttttttggctctagccccaactggtactttgatgtgtggggccagggcaccctggtcacagtctccagtgcctcaaccaaggggccctctgtttttcccctggcaccctgctccagatcaacaagtgagagcactgctgccttgggctgccttgtgaaggactacttccctgagcctgttacagtctcttggaactctggggccctcacctcaggtgtgcataccttcccagctgtcctgcagtcctcagggctttactcattatcctcagtagttactgttccttccagtaattttgggacccagacctatacttgcaatgtggaccacaagcctagcaacaccaaagtggacaagactgttgaaaggaagtgctgtgtagaatgtcccccttgtccagcccctccagtggctggaccatcagtctttctcttcccccccaagccaaaagacacactgatgatctccaggactccagaggtcacttgtgtggtggtagatgtcagtcaggaggacccagaagttcagttcaactggtatgtggatggagtggaagtgcacaatgcaaaaaccaagcccagggaagagcagtttaactccacctacagggtagtgtctgtgctcactgtgctgcaccaggactggctgaatggcaaggagtataaatgtaaggtttccaacaaaggcctgcccagcagcattgagaagactataagcaaggccaaaggtcagcccagagagccccaagtgtacacattgcctccctcccaggaagagatgacaaagaaccaggtgtctctaacctgcctggtcaaaggcttctatccatcagacattgctgtagagtgggagtccaatggacaacctgaaaataattataagacaacccctcctgtgcttgattctgatggcagcttcttcctgtatagcaggctaacagttgacaaaagtagatggcaggagggtaatgtcttctcctgcagtgtcatgcatgaagcactccacaaccattacactcagaagagcttaagcctcagcctggggaaa

SEQ ID NO: 195 - DNA, 1344 bp, synthetic construct
  source 1..1344: mol_type = other DNA
caagtgcagctggtgcagagtggagctgaggtgaagaagccaggagccagtgtaaaggtctcctgcaaagcatctggctacattttcagtaactactggatccaatgggtcaggcaagccccagggcaaggattggaatggatgggggagatcttgccaggttctggaagcacagaatatacagagaactttaaggatagagtgaccatgaccagagacaccagcacttccacagtctacatggagctgtcctctctcagatctgaagatacagcagtctactactgtgccagatatttttttggctctagccccaactggtactttgatgtgtggggccagggcaccctggtcacagtctccagtgcctcaaccaaggggccctctgtttttcccctggcaccctgctccagatcaacaagtgagagcactgctgccttgggctgccttgtgaaggactacttccctgagcctgttacagtctcttggaactctggggccctcacctcaggtgtgcataccttcccagctgtcctgcagtcctcagggctttactcattatcctcagtagttactgttccttccagtaattttgggacccagacctatacttgcaatgtggaccacaagcctagcaacaccaaagtggacaagactgttgaaaggaagtgctgtgtagaatgtcccccttgtccagcccctccagtggctggaccatcagtctttctcttcccccccaagccaaaagacacactgatgatctccaggactccagaggtcacttgtgtggtggtagatgtcagtcaggaggacccagaagttcagtttaattggtatgtggatggagtggaagtgcacaatgcaaaaaccaagcccagggaagagcagtttaactccacctacagggtagtgtctgtgctcactgtgctgcaccaggactggctgaatggcaaggagtataaatgtaaggtttccaacaaaggcctgcccagcagcattgagaagactataagcaaggccaaaggacagcccagagagccccaagtgtacacattgcctccctcccaagaagagatgacaaagaaccaggtgtctctaacctgcctggtcaaaggcttctatccatcagacattgctgtagagtgggagtccaatggacaacctgaaaataattataagacaacccctcctgtgcttgattctgatggcagcttcttcctgtatagcaggctaacagttgacaaaagtagatggcaggagggcaatgtcttctcctgcagtgtcatgcatgaagcactccacaaccattacactcagaagagcttaagcctcagcctggggaaa

SEQ ID NO: 196 - DNA, 1344 bp, synthetic construct
  source 1..1344: mol_type = other DNA
caggtgcagctggtccagtctggagcagaagtcaagaagccaggtgcctcagtcaaagttagctgcaaggcatctggctacatcttcagtaattactggatccagtgggtgaggcaagcccctggccaaggcctggagtggatgggagaaatattacctgggagtggcagcacagagtatacagagaacttcaaagacagggtaaccatgacaagagacaccagcacctccacagtgtacatggagctgagctcccttagatctgaggacacagctgtgtattattgtgccagatatttttttggatctagtcccaactggtactttgatgtctggggacaaggtacattggtaactgtctcatcagctagcactaaaggaccttctgtttttcctttggctccatgttccaggtccacttctgaaagtactgcagccctgggctgcctggtgaaggactacttccctgagcctgtgactgtaagctggaattcaggtgctctcacctctggtgttcatacttttccagctgtcctgcagagctctgggctttattcacttagttcagtagtgacagttccaagctctaactttggcactcagacatatacctgcaatgtggaccacaagcccagcaacaccaaggtggacaaaacagtagaaaggaaatgctgtgtggaatgtccaccctgccctgcccccccagttgctgggcccagtgtatttctcttcccccccaaacccaaggatactctgatgatttcaagaaccccagaggtcacctgtgttgtggttgatgtgtctcaagaagatccagaagttcagtttaattggtatgtggatggggtggaggtgcataatgccaagacaaagcctagagaagagcagttcaacagcacctatagagtagtcagtgtgctcactgtcttgcaccaggactggctgaatggcaaggagtacaagtgcaaagtgtccaacaagggcctaccctcctccattgaaaagaccatctccaaagcaaaaggtcagcccagagagccccaggtctacacactgccaccttcccaggaggagatgaccaaaaatcaagtgtctctaacttgtcttgtgaaaggcttttatccttcagatattgcagttgaatgggagagtaatgggcagcctgaaaacaactacaaaaccacaccaccagtgctggacagtgatggaagcttcttcctgtacagcaggttgactgtggataaatccaggtggcaggaaggaaatgtcttctcctgcagtgtgatgcatgaagccctccacaaccactacacccagaagagcttatccctctcattagggaag

SEQ ID NO: 197 - DNA, 1344 bp, synthetic construct
  source 1..1344: mol_type = other DNA
caggtgcagctggtccagtctggagcagaagtcaagaagccaggtgcctcagtcaaagttagctgcaaggcatctggctacatcttcagtaattactggatccagtgggtcaggcaagcccctggccaaggcctggagtggatgggagaaatattacctgggagtggcagcacagagtatacagagaacttcaaagacagggtaaccatgacaagagacaccagcacctccacagtgtacatggagctgagctcccttagatctgaggacacagctgtgtattattgtgccagatatttttttggatctagtcccaactggtactttgatgtctggggacaaggcacattggtaactgtctcatcagctagcactaaaggaccttctgtttttcctttggctccatgttccaggtccacttctgaaagtactgcagccctgggctgcctggtgaaggactacttccctgagcctgtgactgtaagctggaattcaggtgctctcacctctggtgttcatacttttccagctgtcctgcagagctctgggctttattcacttagttcagtagtgacagttccaagctctaactttggcactcagacatatacctgcaatgtggaccacaagcccagcaacaccaaggtggacaaaacagtagaaaggaaatgctgtgtggaatgtccaccctgccctgcccccccagttgctgggcccagtgtatttctcttcccccccaaacccaaggatactctgatgatttcaagaaccccagaggtcacctgtgttgtggttgatgtgtctcaagaagatccagaagttcagtttaattggtatgtggatggggtggaggtgcataatgccaagacaaagcctagagaagagcagttcaacagcacctatagagtagtcagtgtgctcactgtcttgcaccaggactggctgaatggcaaggagtacaagtgcaaagtgtccaacaagggcctaccctcctccattgaaaagaccatctccaaagcaaagggccagcccagagagccccaggtctacacactgccaccttcccaggaggagatgaccaaaaatcaagtgtctctaacttgtcttgtgaaaggcttttatccttcagatattgcagttgaatgggagagtaatgggcagcctgaaaacaactacaaaaccacaccaccagtgctggacagtgatggaagcttcttcctgtacagcaggttgactgtggataaatccaggtggcaggaaggaaatgtcttctcctgcagtgtgatgcatgaagccctccacaaccactacacccagaagagcttatccctctcattagggaag

SEQ ID NO: 198 - DNA, 1344 bp, synthetic construct
  source 1..1344: mol_type = other DNA
caagtacagctggtccagagtggagctgaagtgaagaagccaggggcctcagtcaaggtgagctgcaaggcatctggttacatcttttccaactactggatccagtgggtgaggcaagcccctgggcaaggcctggagtggatgggggagattcttcctgggtctggctccactgaatatacagaaaacttcaaggatagagtgaccatgaccagagacacctccacctctacagtgtacatggagctgtccagccttaggtcagaagatacagcagtctactactgtgccagatacttctttggcagcagccccaactggtactttgatgtctggggccagggcaccttggtgacagtgtcttctgcctcaactaaaggaccatctgtcttcccattagccccctgcagcaggtccacatctgagagcacagcagccctgggctgcctggtgaaagactattttcctgagcctgtgactgtttcatggaatagtggtgctttgacaagtggggtgcacaccttccctgcagtactgcagagcagtggcctttattccctgtcatctgtggtcacagtaccatctagcaactttggaacccagacctacacttgcaatgtggaccacaagcccagcaatacaaaagtggacaaaactgtggagagaaaatgctgtgttgagtgtcccccatgccctgcccccccagttgcaggcccttcagtctttctattcccccctaagcccaaggacacactcatgattagtaggactccagaagtcacctgtgtggtggttgatgtttcccaggaggaccctgaggtgcagttcaactggtatgttgatggagtagaagttcataatgctaaaaccaagcctagagaggagcagtttaacagcacttatagagttgtttctgtgctcactgtcctccatcaggactggctgaatggaaaggagtataaatgtaaggtgagtaacaaaggtttgccctccagcatagagaagactatcagcaaagccaaaggtcagcccagggagccacaagtctatactctccccccatcacaagaagaaatgacaaaaaaccaggtgtccttgacctgccttgtgaagggcttctacccctcagatattgctgtggaatgggagagtaatgggcagccagaaaataattataagaccacccctccagtactagactcagatggttccttcttcctgtatagcaggctgacagtagacaagtccagatggcaggaagggaatgtcttttcatgttctgtcatgcatgaagctctgcacaaccactacacccagaagagtctctccctcagtttaggaaaa

SEQ ID NO: 199 - DNA, 1344 bp, synthetic construct
  source 1..1344: mol_type = other DNA
caagtacagctggtccagagtggagctgaagtgaagaagccaggggcctcagtcaaagtcagctgcaaggcatctggttacatcttttccaactactggattcaatgggtcagacaagcccctgggcaaggcctggagtggatgggggagattcttcctgggtctggctccactgaatatacagaaaacttcaaggatagagtgaccatgaccagagacacctccacctctacagtgtacatggagctgtccagccttaggtcagaagatacagcagtctactactgtgccagatacttctttggcagcagccccaactggtattttgatgtctggggccagggcaccttggtgacagtgtcttctgcctcaactaaaggaccatctgtcttcccattagccccctgcagtaggtccacatctgagagcacagcagccctgggctgcctggtgaaagactattttcctgagcctgtgactgtttcatggaatagtggtgctttgacaagtggggtgcacaccttccctgcagtactgcagagcagtggcctttattccctgtcatctgtggtcacagtaccatctagcaactttggaacccagacctacacttgcaatgtggaccacaagcccagcaatacaaaagtggacaaaactgtggagagaaaatgctgtgttgagtgtcccccatgccctgcccccccagttgcaggcccttcagtctttctattcccccctaagcccaaggacacactcatgattagtaggactccagaagtcacctgtgtggtggttgatgtttcccaggaggaccctgaggtgcaatttaattggtatgttgatggagtagaagttcataatgctaaaaccaagcctagagaggagcagtttaacagcacttatagagttgtttctgtgctcactgtcctccatcaggactggctgaatggaaaggagtataaatgcaaagtcagtaacaaaggcttgccctccagcatagagaagactatcagcaaagccaaaggccagcccagggagccacaagtctatactctccccccatcacaagaagaaatgacaaaaaaccaggtgtccttgacctgccttgtgaagggcttctacccctcagatattgctgtggaatgggagagtaatgggcagccagaaaataattataagaccacccctccagtactagactcagatggttccttcttcctgtatagcaggctgacagtagacaagtccagatggcaggaagggaatgtcttttcatgttctgtcatgcatgaagctctgcacaaccactacacccagaagagtctctccctcagtttaggaaaa

SEQ ID NO: 200 - DNA, 642 bp, synthetic construct
  source 1..642: mol_type = other DNA
gatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgc

SEQ ID NO: 201 - DNA, 642 bp, synthetic construct
  source 1..642: mol_type = other DNA
gacatccagatgacacagagcccttccagcctctctgcctcggtgggggacagagtcaccatcacctgtggggcctcggagaacatctatggtgctctgaactggtaccagcagaaacctgggaaggcccccaagctgctcatctatggggccaccaacctggctgatggtgtgccctcgcgcttctctggatctggaagtggcacagacttcaccctcaccatctccagcctgcaacctgaggactttgccacctactactgccagaatgtgctgaacacacccctcacttttggacaagggaccaaagtagaaatcaagaggacagtggcggcgcccagtgtcttcatcttccctccaagcgatgaacaactaaagtctggaacagcctcggtggtctgcctgctgaacaacttctacccaagagaggccaaggtgcagtggaaagttgataatgccctgcagagtggaaacagccaggagagtgtcactgaacaagacagcaaggacagcacctacagcctcagcagcaccctcactttatcaaaagctgactatgagaagcacaaagtttatgcctgtgaagtaactcaccagggcctcagctcccctgtcaccaagtccttcaaccgtggggagtgt

SEQ ID NO: 202 - DNA, 642 bp, synthetic construct
  source 1..642: mol_type = other DNA
gatatccagatgacacagagccccagctccttatcagcctcggtgggggacagggtcaccatcacctgtggggcctcggagaacatctatggggctctgaactggtaccagcagaaaccagggaaggcccccaagctgctgatctatggggccaccaacctggctgatggggtgccttcacgcttctctggcagtggcagtggaactgacttcaccctgaccatcagcagcttacaaccagaggattttgccacctactactgccagaatgtcctcaacacacctctcacttttggacaaggaaccaaagtggaaatcaagagaacagtggctgcacccagtgtgttcatcttcccaccaagcgatgaacaactaaagagtggaacagcatctgtggtgtgcctgctcaacaacttctacccaagagaggccaaggtgcagtggaaggttgacaatgccctgcagagtggaaacagtcaagaatctgtcactgaacaagacagcaaggacagcacctacagcctgagctccaccttaactttatcaaaagctgactatgagaagcacaaggtctatgcctgtgaagtgacacaccagggcctctccagccctgtcaccaaatcttttaacagaggagaatgt

SEQ ID NO: 203 - DNA, 642 bp, synthetic construct
  source 1..642: mol_type = other DNA
gatatccagatgacccagagccccagctccctgtctgcatctgtaggtgacagggtcaccattacctgtggagcatcagaaaacatctatggggccttgaactggtatcagcagaagccaggcaaagccccaaagctgttgatatatggtgccaccaacttggcagatggtgtgccaagcagattcagtggatcaggcagtggcacagatttcacactgaccattagcagcctgcaacctgaagactttgctacctactactgccagaatgttctgaacacccccctgacctttggccagggcaccaaggtggagatcaagaggactgttgctgccccttctgtattcatcttcccacccagtgatgagcaattgaagtcaggcactgcatcagtggtgtgtcttcttaacaacttctaccccagagaggccaaggtacaatggaaggttgacaatgcacttcagagtggaaacagccaggagtcagtcactgaacaggacagcaaggatagcacatacagcctgtctagcaccctgactctgagcaaggctgactatgagaagcataaggtgtatgcctgtgaggttacccaccagggactgagcagccctgtgacaaaaagcttcaataggggggagtgc

SEQ ID NO: 204 - DNA, 642 bp, synthetic construct
  source 1..642: mol_type = other DNA
gacatccagatgacacagtctccaagctccctgtctgccagcgtgggagatagggtgaccatcacatgcggggccagcgagaacatttacggcgccctgaattggtatcagcagaagccaggcaaagcccccaagctgctgatctacggagcaactaacctggcagacggagtgccatccagattctccggctctggaagcgggactgactttactctgaccatttctagcctgcagcctgaagatttcgccacctactattgccagaacgtgctgaataccccactgacatttggccagggaactaaagtggagatcaagagaaccgtggccgcccccagcgtgttcatttttcccccttccgacgaacagctgaaatccggaaccgcctctgtggtgtgtctgctgaacaatttctaccctagggaggccaaagtgcagtggaaggtggataacgccctgcagtccggcaattctcaggagagcgtgacagaacaggactccaaagattctacttatagcctgtcctctacactgactctgtccaaggccgattacgagaagcacaaagtgtatgcctgcgaagtgacccatcagggcctgagctcccccgtgacaaagtcttttaatagaggagaatgt

SEQ ID NO: 205 - DNA, 642 bp, synthetic construct
  source 1..642: mol_type = other DNA
gacatccagatgacccagtctccaagctccctgtctgccagcgtgggagatagggtgaccatcacatgcggcgccagcgagaacatttacggcgccctgaattggtatcagcagaagccaggcaaagcccccaagctgctgatctacggagcaacaaacctggcagacggagtgccatccaggttctccggctctggcagcggcaccgactttaccctgacaatttctagcctgcagcctgaggatttcgccacatactattgccagaacgtgctgaataccccactgacatttggccagggcaccaaagtggagatcaagagaacagtggccgcccccagcgtgttcatttttcccccttccgacgagcagctgaaatccggcaccgcctctgtggtgtgtctgctgaacaatttctaccctagggaggccaaagtgcagtggaaggtggataacgccctgcagtccggcaattctcaggagagcgtgaccgagcaggactccaaagattctacatatagcctgtcctctaccctgacactgtccaaggccgattacgagaagcacaaagtgtatgcatgcgaggtgacccaccagggactgagctcccctgtgacaaagtcttttaatagaggcgagtgt

SEQ ID NO: 206 - DNA, 642 bp, synthetic construct
  source 1..642: mol_type = other DNA
gatattcagatgacccagtcccccagcagcctgtcagccagtgtcggagaccgcgtcaccattacctgtggagcctcagagaacatttacggcgccctgaactggtaccagcagaagcccggcaaggcccctaagctgctgatctacggagctaccaatctggctgacggagtgccttccagattcagcggatccggaagcggaaccgactttaccctgaccatcagctccctgcagcccgaggatttcgccacctactactgccagaacgtgctgaatacccctctgacctttggccagggcaccaaggtggagatcaagagaaccgtggccgccccctccgtgttcatctttccccctagcgacgagcagctgaagtccggcaccgccagcgtggtgtgtctgctgaacaatttctaccctcgcgaggccaaggtgcagtggaaggtggataacgccctgcagtccggcaatagccaggagtccgtgaccgagcaggacagcaaggattccacctacagcctgagctccaccctgaccctgagcaaggccgattacgagaagcacaaggtgtacgcctgcgaggtcacccaccaggggctgtcttcacccgtcaccaaatctttcaatagaggagagtgc

SEQ ID NO: 207 - DNA, 642 bp, synthetic construct
  source 1..642: mol_type = other DNA
gacatccagatgacccagtcccctagctccctgagcgcctccgtgggagatagagtgaccatcacctgcggagcaagcgagaacatctacggagcactgaattggtaccagcagaagcccggcaaggcccctaagctgctgatctacggagcaaccaacctggcagacggagtgccttccaggttcagcggatccggaagcggaaccgactttaccctgaccatcagctccctgcagcccgaggatttcgccacctactactgccagaacgtgctgaatacccctctgacctttggccagggcaccaaggtggagatcaagagaaccgtggccgccccctccgtgttcatctttccccctagcgacgagcagctgaagtccggcaccgccagcgtggtgtgtctgctgaacaatttctaccctagggaggccaaggtgcagtggaaggtggataacgccctgcagtccggcaatagccaggagtccgtgaccgagcaggacagcaaggattccacctacagcctgagctccaccctgaccctgtccaaggccgattacgagaagcacaaggtgtacgcatgcgaggtgacccaccagggactgagctcccctgtgaccaagagctttaatagaggcgagtgt

SEQ ID NO: 208 - DNA, 642 bp, synthetic construct
  source 1..642: mol_type = other DNA
gacatccagatgacacagtctccaagctccctgtctgccagcgtgggagatagggtgaccatcacatgcggggccagcgagaatatttacggcgccctgaactggtatcagcagaagccaggcaaagcccctaagctgctgatctacggagccactaatctggccgacggggtgcctagcagattctccggctctggaagcgggactgactttactctgaccatttctagcctgcagcccgaagatttcgccacctactattgccagaatgtgctgaacaccccactgacatttggccagggaactaaagtggagatcaagagaaccgtggccgcccctagcgtgttcatttttcctccctccgacgaacagctgaaatccggaaccgcctctgtggtgtgtctgctgaataacttctaccccagggaggccaaagtgcagtggaaggtggataatgccctgcagtccggcaactctcaggagagcgtgacagaacaggactccaaagattctacttatagcctgtcctctacactgactctgtccaaggccgattacgagaagcacaaagtgtatgcctgcgaagtgacccatcagggcctgagctcccctgtgacaaagagctttaacagaggagaatgt

SEQ ID NO: 209 - DNA, 642 bp, synthetic construct
  source 1..642: mol_type = other DNA
gacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgc

SEQ ID NO: 210 - DNA, 642 bp, synthetic construct
  source 1..642: mol_type = other DNA
gacatccagatgacccagagccccagcagcctatcagcttctgtaggggacagggtcaccatcacctgtggagcatcagagaacatatatggggccctgaactggtaccagcagaaaccaggcaaggcccccaagctgctgatttatggtgccaccaacctggcagatggtgttccttccaggtttagtggctctggaagtggaacagacttcaccctcaccatttcctccctgcagccagaagattttgccacttactactgccagaatgtgctgaatactcctttgacatttggccaagggactaaagtggagatcaagaggacagtagctgctccttctgtcttcatcttccccccctcagatgaacagctgaagtctggcacagccagtgtggtgtgcctgctcaacaacttctacccaagagaagcaaaagtgcagtggaaggtggacaatgcccttcaatctgggaattcccaggagagtgtcacagagcaggacagcaaagattccacctacagcctctccagcacacttactttatcaaaagctgattatgagaagcacaaggtctatgcctgtgaagttactcatcaaggcttgagctcccctgtgaccaagagcttcaacagaggagagtgc

SEQ ID NO: 211 - DNA, 642 bp, synthetic construct
  source 1..642: mol_type = other DNA
gacatccagatgacccagagccccagcagcctatcagcttctgtaggggacagggtcaccatcacctgtggagcatcagagaacatatatggggccctgaactggtaccagcagaaaccaggcaaggcccccaagctgctgatttatggtgccaccaacctggcagatggtgttccttccaggtttagtggctctggaagtggaacagacttcaccctcaccatttccagcctgcagccagaagattttgccacttactactgccagaatgtgctgaatactcctttgacatttggccaagggactaaagtggagatcaagaggacagtagctgctccttctgtcttcatcttccccccctctgatgaacagctgaagtctggcacagccagtgtggtgtgcctgctcaacaacttctacccaagagaagcaaaagtgcagtggaaggtggacaatgcccttcaatctgggaattcccaggagagtgtcacagagcaggacagcaaagattccacctacagcctctccagcacacttactttatcaaaagctgattatgagaagcacaaggtctatgcctgtgaagttactcatcaaggcttgagctcccctgtgaccaagagcttcaacagaggagagtgc

SEQ ID NO: 212 - DNA, 642 bp, synthetic construct
  source 1..642: mol_type = other DNA
gacatccagatgacccagagcccctcttccttatcagcatcagttggagatagagtcaccattacttgtggggcttcagaaaatatatatggtgccctcaactggtaccagcagaagccaggcaaagcccccaagctgctcatttatggggccaccaacctggctgatggtgtgcccagtaggttttctggctctggaagtggaacagacttcaccctaaccatctccagccttcaacctgaagattttgcaacttactactgccagaatgtgctgaatacaccacttacatttggccaaggcaccaaggtggagatcaagaggactgtagctgctcctagtgttttcatttttcctccaagtgatgaacagctgaaatcaggcactgcatctgtggtctgcctgctgaacaacttctaccccagggaggccaaggtccagtggaaggtggacaatgccctgcagagtgggaatagccaggagtctgttacagagcaggactccaaggacagcacatacagcctctcctctactttgaccttgtccaaagcagattatgagaagcacaaagtatatgcctgtgaggtgacacatcaagggctcagcagccctgtgaccaaatccttcaacagaggagaatgc

SEQ ID NO: 213 - DNA, 642 bp, synthetic construct
  source 1..642: mol_type = other DNA
gacatccagatgacccagagcccctcttccttatcagcatcagttggagatagagtcaccattacttgtggggcttcagaaaatatatatggtgccctcaactggtaccagcagaagccaggcaaagcccccaagctgctcatttatggggccaccaacctggctgatggtgtgccctccaggttttctggctctggaagtggaacagacttcaccctaaccatctcctcccttcaacctgaagattttgcaacttactactgccagaatgtgctgaatacaccacttacatttggccaaggcaccaaagttgagatcaagaggactgtagctgctcctagtgtgttcatcttccctccatctgatgaacagctgaaatcaggcactgcatctgtggtctgcctgctgaacaacttctaccccagggaggccaaggtccagtggaaggtggacaatgccctgcagagtgggaatagccaggagtctgttacagagcaggactccaaggacagcacatacagcctctcctctactttgaccttgtccaaagcagattatgagaagcacaaagtatatgcctgtgaggtgacacatcaagggctcagcagccctgtgaccaaatccttcaacagaggagaatgc

SEQ ID NO: 214 - DNA, 642 bp, synthetic construct
  source 1..642: mol_type = other DNA
gacatccagatgacccaaagccccagctccttgtcagcttcagttggtgacagggtaacaattacttgtggggcttcagaaaatatttatggagcactcaactggtaccagcagaagccaggcaaggccccaaagttgttaatatatggggccacaaacctggcagatggggtaccctccaggttttctggttcaggcagtgggacagacttcaccctcaccatcagcagtcttcaaccagaagattttgccacctactactgccagaatgtgctgaatactcctcttacatttggacaaggcaccaaggtggagattaaaagaactgtggctgcacccagtgtcttcatctttcctccttcagatgaacagctgaagagtggcacagcatctgtggtgtgcctgctgaacaacttctacccaagagaggccaaggtccagtggaaagtggacaatgccctgcaaagtggaaattcccaggagtctgttacagagcaggacagcaaagattctacttacagcctaagcagcactttaactctttccaaggctgattatgaaaaacataaagtttatgcctgtgaggtgacccaccagggcctctcctctcctgtcaccaaatccttcaacagaggagaatgc

SEQ ID NO: 215 - DNA, 642 bp, synthetic construct
  source 1..642: mol_type = other DNA
gacatccagatgacccaaagccccagctccttgtcagcttcagttggtgacagggtaacaattacttgtggggcttcagaaaatatttatggagcactcaactggtaccagcagaagccaggcaaggccccaaagttgttaatatatggggccacaaacctggcagatggggtaccctccaggttttctggttcaggcagtgggacagacttcaccctcaccatcagcagtcttcaaccagaagattttgccacctactactgccagaatgtgctgaatactcctcttacatttggacaaggcaccaaggtggagattaaaagaactgtggctgcacccagtgtcttcatctttcctccttctgatgaacagctgaagagtggcacagcatctgtggtgtgcctgctgaacaacttctacccaagagaggccaaggtccagtggaaagtggacaatgccctgcaaagtggaaattcccaggagtctgttacagagcaggacagcaaagattctacttacagcctaagcagcactttaactctttccaaggctgattatgaaaaacataaagtttatgcctgtgaggtgacccaccagggcctctcctctcctgtcaccaaatccttcaacagaggagaatgc

SEQ ID NO: 216 - DNA, 642 bp, synthetic construct
  source 1..642: mol_type = other DNA
gacatccagatgacccagagtcccagtagtctatcagcctctgtaggggatagagttaccatcacctgtggggcttcagaaaatatatatggggccctcaactggtaccagcagaaaccaggaaaagcacccaagctcttgatttatggagccacaaatttggcagatggagtacccagcaggttttctggttctggctcaggcactgacttcaccttaactatttcctccctgcagcctgaggactttgcaacttactactgccagaatgtgctgaatacacctcttacctttggccaagggacaaaagtggagatcaagaggactgtggctgcaccatctgtcttcatttttcccccttcagatgaacagctgaagagtggtacagcttctgtggtgtgcctgctgaacaacttctacccaagagaagccaaggttcagtggaaggtggacaatgccctgcaaagtggcaacagccaggagtcagtcacagagcaggacagcaaagattccacctacagcctctccagcactcttacactttccaaagctgattatgagaagcacaaagtttatgcctgtgaggtgactcatcaaggcctgagcagccctgtcaccaagtccttcaacagaggagaatgc

SEQ ID NO: 217 - DNA, 642 bp, synthetic construct
  source 1..642: mol_type = other DNA
gacatccagatgacccagagtcccagtagtctatcagcctctgtaggggatagagttaccatcacctgtggggcttcagaaaatatatatggggccctcaactggtaccagcagaaaccaggaaaagcacccaagctcttgatttatggagccacaaatttggcagatggagtacccagcaggttttctggttctggctcaggcactgacttcaccttaactatttcctccctgcaacctgaggactttgcaacttactactgccagaatgtgctgaatacacctcttacctttggccaagggacaaaagtggagatcaagaggactgtggctgcaccatctgtcttcatttttcccccttctgatgaacagctgaagagtggtacagcttctgtggtgtgcctgctgaacaacttctacccaagagaagccaaggttcagtggaaggtggacaatgccctgcaaagtggcaacagccaggagtcagtcacagagcaggacagcaaagattccacctacagcctctccagcactcttacactttccaaagctgattatgagaagcacaaagtttatgcctgtgaggtgactcatcaaggcctgagcagccctgtcaccaagtccttcaacagaggagaatgc

SEQ ID NO: 218 - DNA, 370 bp, synthetic construct
  source 1..370: mol_type = other DNA
gggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccacc

SEQ ID NO: 219 - DNA, 4483 bp, synthetic construct
  source 1..4483: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggttgtgcagggcttcgtgcatcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtagagccagatcctggcaggatctcgcccatccattccaggccttggccaggggcctgccgcacccactggatccagtagttgctgaagatatagccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 220 - DNA, 171 bp, synthetic construct
  source 1..171: mol_type = other DNA
ccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacag

SEQ ID NO: 221 - DNA, 3677 bp, synthetic construct
  source 1..3677: mol_type = other DNA
ccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 222 - DNA, 4058 bp, synthetic construct
  source 1..4058: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgccttgctgtcctgccccaccccaccccccagaatagaatgacacctactcagacaatgtgatgcaatttcctcattttattaggaaaggacagtgggagtggcaccttccagggtcaaggaaggcaagggggaggggcaaacaacagatggctggcaactagaaggcacagtcatttgcccagagacaggctcaggcttttctgggtgtagtggttgtgcagggcctcatgcatcacagagcagctgaaaacattgccctcttgccatctgctcttgtccactgtcagcctgctgtacaggaagaagctgccatcagagtccagcacaggtggggtggtcttatagttgttctcaggctggccattgctttcccattccactgcaatgtctgaggggtagaagcccttcaccaggcaggtcaggctcacctggttttttgtcatttcctcttggctaggaggcagggtatacacctgaggctctctgggttggcccttggccttgctgatggttttttctatgctgctggggaggcccttgttggacactttacatttgtactccttgccattgagccagtcctggtgcagaacagttagcactgacaccactctgtaggtgctgttgaactgctcctcccttggcttggtcttggcattgtgaacctccactccatccacataccagttaaactggacctcagggtcttcctggctcacatcaaccaccacacaggtcacttctggggttctggagatcatcagggtgtccttgggtttgggggggaacaggaacacagaggggccagccacaggaggggcagggcagggggggcattccacacagcacttcctctccactgtcttgtccaccttggtgttgctgggcttgtggtccacattgcaggtgtaggtctgtgtgccaaagttgctgcttggcactgtcaccacagagctcagggagtacaggccactgctttgcaggacagcagggaaggtgtgtactccagaggtcagggctccagagttccagctaacagtcacaggttcagggaagtaatccttcaccaggcagcccagggcagctgttgactcagatgtgcttctgctgcaaggggccagagggaagacagaagggcccttggtgctggcagagctcactgtcaccagggtgccctggccccacacatcaaagtaccagttagggctgctgccaaagaagtatctggcacagtagtacacagctgtgtcctcagatctcaggctggacagttccatgtacactgtggaggtgctggtatctctggtcattgtcactctgtccttgaagttctctgtgtactctgtgctgccagagccaggcaggatttctcccatccactccaggccttgaccaggggcctgtctgacccactggatccagtagttgctgaagatgtagccagaggccttgcaggacaccttcacactggctccaggcttcttcacctcagctccagactgcaccagctgcacctggctgtggacacctgtggctgtggccaccaggaagaggatgatgcagctccagcccatggtggctcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattgaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggacctattaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagaagctttattgaggtagtttatcacagttaaattgctaaggcagtcagtgcttctgacacaacagtcttgaacttaagctgcagtgactctcttaaggtagccttgcagaagttggtcgtgaggcactgggcaggtaagtatcaaggttacaagacaggtttaaggagaccaatagaaactgggcatgtgtagacagagaagactcttgggtttctgataggcacctattggtcttactgacatccactttgcctttctctccacaggtgtccactcccagttcaattacagctcttaaggccctgcagaccggtgccaccatggacatgagggtgccagcacagctgctgggcctcctgctgctgtggctgagtggtgcaagatgtgacatccagatgacacagagcccctccagcctgagtgcctcagtgggtgacagagtgaccattacatgtggagcctctgagaacatctatggagccctgaactggtaccagcagaagcctggcaaggcccctaagctgctgatctatggggccaccaacctggctgatggagtgcctagcaggttcagtggctctggctctggcacagacttcaccctcaccatcagcagcctgcagcctgaagactttgccacctactactgccagaatgtgctgaacacccccctgacctttggccagggcaccaaagttgaaatcaagagaacagtggctgccccttctgtgttcatcttccctccctctgatgagcagctgaagtctggcacagcctctgtggtgtgcctgctgaacaacttctacccaagagaggccaaggtccaatggaaagtggacaatgccctgcagtctggcaacagccaggagtcagtgacagaacaggatagcaaggacagcacctacagcctgagcagcaccctgacactgagcaaagctgactatgagaagcacaaggtgtatgcctgtgaagtgacccaccaaggcctgagcagcccagtgaccaagagcttcaacagaggagagtgctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2023/0374546 A1
(43) Pub. Date: Nov. 23, 2023

(54) BIDIRECTIONAL DUAL PROMOTER EXPRESSION VECTORS AND USES THEREOF

(71) Applicant: Homology Medicines, Inc., Bedford, MA (US)

(72) Inventors: Carmen BARNES, Bedford, MA (US); Yogeshwar Sharma, Bedford, MA (US); Omar Francone, Bedford, MA (US); Hillard Rubin, Bedford, MA (US); Wei Wang, Bedford, MA (US); Gustavo Cerqueira, Bedford, MA (US); Serena Nicole Dollive, Bedford (OM); Andrew Pla, Bedford, MA (US)

(21) Appl. No.: 18/182,001

(22) Filed: Mar. 10, 2023

Related U.S. Application Data
(60) Provisional application No. 63/269,192, filed on Mar. 11, 2022.

(51) Int. Cl. C12N 15/86; C07K 16/18; A61K 48/00; A61P 37/06

(52) U.S. Cl. CPC C12N 15/86; C07K 16/18; A61K 48/0066; A61K 48/0041; A61P 37/06; C12N 2750/14021; C12N 2750/14043; C12N 2750/14052; C12N 2750/14071; C12N 2830/50; C12N 2830/008; A61K 2039/505

(57) ABSTRACT

Provided herein are polynucleotides comprising novel bidirectional dual expression cassettes, recombination adeno-associated virus (rAAV) comprising these polynucleotides, and methods of making and using the polynucleotides and rAAV. Also provided are novel transcriptional control elements (e.g., promoters, enhancers, introns, polyadenylation sequences, and combinations thereof), and novel antibody coding sequences. The compositions and methods disclosed herein are particularly advantageous in that they allow for the efficient expression of two different polypeptides (e.g., an antibody heavy chain and an antibody light chain) in a cell. In particular, they allow for the efficient expression of antibodies (e.g., anti-C5 antibodies) in a subject, for the treatment of diseases (e.g., C5-mediated diseases, such as PNH).

### RELATED APPLICATIONS

This application claims priority to U.S. Provisional Patent Application Ser. No. 63/269,192, filed Mar. 11, 2022, the entire disclosure of which is hereby incorporated herein by reference.

### SEQUENCE LISTING

This application contains a sequence listing which has been submitted electronically and in ST.26 format is hereby incorporated by reference in its entirety (said ST.26 copy, created on Mar. 9, 2023, is named “HMW-053US.XML” and is 524,319 bytes in size).

### BACKGROUND

Therapeutic antibodies represent a potent class of drugs, possessing high specificity to a target of interest. However, many antibodies require large individual doses and regular administration to achieve the desired therapeutic effect. This is especially true for antibody targets that are found at high concentrations in a patient's serum. For example, anti-complement component 5 (C5) antibodies used for the treatment of C5-mediated diseases, such as paroxysmal nocturnal hemoglobinuria (PNH), neuromyelitis optica spectrum disorder (NMOSD), and atypical hemolytic uremic syndrome (aHUS) require multiple large doses of the antibody due to the high abundance of C5 in serum.

Viral delivery mechanisms offer an attractive alternative to conventional antibody treatments, especially for antibody targets that are found at high concentrations in a patient's serum. In particular, a single administration of a viral vector harboring capable of expressing high levels of antibody heavy and light chains has the potential to produce sustained therapeutic levels of an antibody in the serum of a subject, thereby bypassing the need for continual administration of high dose antibody.

Accordingly, there is a need in the art for improved viral vectors for the efficient and sustained expression of antibodies in a subject.

### SUMMARY

Provided herein are polynucleotides comprising novel bidirectional dual expression cassettes, recombination adeno-associated virus (rAAV) comprising these polynucleotides, and methods of making and using these polynucleotides and rAAV. Also provided are novel transcriptional control elements (e.g., promoters, enhancers, introns, polyadenylation sequences, and combinations thereof), and novel antibody coding sequences. The compositions and methods disclosed herein are particularly advantageous in that they allow for the efficient, simultaneous expression of two different polypeptides (e.g., an antibody heavy chain and an antibody light chain) in a cell. In particular, they allow for the efficient and sustained expression of antibodies (e.g., anti-C5 antibodies) in a subject, for the treatment of diseases (e.g., C5-mediated diseases, such as PNH).

Gene therapy vectors intended for systemic expression of full-length monoclonal antibodies require the regulated transcription of two independent transgenes (one for the antibody heavy chain and one for the antibody light chain). Previous designs of rAAV for antibody expression have typically employed a single promoter that drives expression of a single bicistronic transgene encoding both the antibody heavy chain and the antibody light chain, separated by a ribosomal skipping element (van den Berg et al., Mol Ther Methods Clin Dev. 2019 Sep. 13; 14: 100-112). In contrast to these prior art rAAV vectors, the vectors disclosed herein employ a novel bidirectional dual promoter unit comprising two divergent promoters positioned back-to-back, transcribing antibody heavy chain and light chain coding sequences in opposing directions. The bidirectional dual promoter vectors disclosed herein position the polyadenylation sequences for each of the antibody heavy chain and light chain expression cassettes adjacent to an ITR. Applicants hypothesize that such positioning offers a superior design to traditional constructs, because the polyadenylation sequences may serve to limit any deleterious transcription initiated by the ITRs themselves.

The instant disclosure includes, without limitation, the specific embodiments set forth below.

Embodiment 1—An isolated polynucleotide comprising from 5′ to 3′: (a) a reverse complement human α1-antitrypsin (hAAT) promoter sequence and a human hepatic control region 1 (hHCR1) sequence; (b) a reverse complement hAAT promoter sequence that is at least 85% identical to SEQ ID NO:17, and an hHCR1 sequence that is at least 85% identical to SEQ ID NO:22; (c) a reverse complement hAAT promoter sequence that is at least 85% identical to SEQ ID NO:17, and a reverse complement hHCR1 sequence that is at least 85% identical to SEQ ID NO:23; (d) a reverse complement hAAT promoter sequence that is at least 85% identical to SEQ ID NO:18, and an hHCR1 sequence that is at least 85% identical to SEQ ID NO:22; (e) a reverse complement hAAT promoter sequence that is at least 85% identical to SEQ ID NO:18, and a reverse complement hHCR1 sequence that is at least 85% identical to SEQ ID NO:23; (f) a reverse complement hAAT promoter sequence that is at least 85% identical to SEQ ID NO:20, and an hHCR1 sequence that is at least 85% identical to SEQ ID NO:22; (g) a reverse complement hAAT promoter sequence that is at least 85% identical to SEQ ID NO:20, and a reverse complement hHCR1 sequence that is at least 85% identical to SEQ ID NO:23; (h) a reverse complement hAAT promoter sequence that is at least 85% identical to SEQ ID NO:17, and an hHCR1 sequence that is at least 85% identical to SEQ ID NO:24; (i) a reverse complement hAAT promoter sequence that is at least 85% identical to SEQ ID NO:18, and an hHCR1 sequence that is at least 85% identical to SEQ ID NO:24; (j) a reverse complement hAAT promoter sequence that is at least 85% identical to SEQ ID NO:18, and a reverse complement hHCR1 sequence that is at least 85% identical to SEQ ID NO:25; (k) a reverse complement hAAT promoter sequence that is at least 85% identical to SEQ ID NO:20, and an hHCR1 sequence that is at least 85% identical to SEQ ID NO:24; or (1) a reverse complement hAAT promoter sequence that is at least 85% identical to SEQ ID NO:20, and a reverse complement hHCR1 sequence that is at least 85% identical to SEQ ID NO:25.

Embodiment 2—The isolated polynucleotide of embodiment 1, comprising from 5′ to 3′: (a) a reverse complement hAAT promoter sequence consisting of SEQ ID NO:17, and an hHCR1 sequence consisting of SEQ ID NO:22; (b) a reverse complement hAAT promoter sequence consisting of SEQ ID NO:17, and a reverse complement hHCR1 sequence consisting of SEQ ID NO:23; (c) a reverse complement hAAT promoter sequence consisting of SEQ ID NO:18, and an hHCR1 sequence consisting of SEQ ID NO:22; (d) a reverse complement hAAT promoter sequence consisting of SEQ ID NO:18, and a reverse complement hHCR1 sequence consisting of SEQ ID NO:23; (e) a reverse complement hAAT promoter sequence consisting of SEQ ID NO:20, and an hHCR1 sequence consisting of SEQ ID NO:22; (f) a reverse complement hAAT promoter sequence consisting of SEQ ID NO:20, and a reverse complement hHCR1 consisting of SEQ ID NO:23; (g) a reverse complement hAAT promoter sequence consisting of SEQ ID NO:17, and an hHCR1 sequence consisting of SEQ ID NO:24; (h) a reverse complement hAAT promoter sequence consisting of SEQ ID NO:18, and an hHCR1 sequence consisting of SEQ ID NO:24; (i) a reverse complement hAAT promoter sequence consisting of SEQ ID NO:18, and a reverse complement hHCR1 sequence consisting of SEQ ID NO:25; (j) a reverse complement hAAT promoter sequence consisting of SEQ ID NO:20, and an hHCR1 sequence consisting of SEQ ID NO:24; or (k) a reverse complement hAAT promoter sequence consisting of SEQ ID NO:20, and a reverse complement hHCR1 sequence consisting of SEQ ID NO:25.

Embodiment 3—The isolated polynucleotide of embodiment 2, comprising a sequence selected from the group consisting of SEQ ID NOs:36-40 and 72.

Embodiment 4—The isolated polynucleotide of any one the preceding embodiments, further comprising a transthyretin (TTR) promoter sequence positioned 3′ to the hHCR1 sequence or the reverse complement hHCR1 sequence.

Embodiment 5—The isolated polynucleotide of embodiment 4, wherein the TTR promoter sequence comprises a nucleic acid sequence that is at least 85% identical to SEQ ID NO:26, optionally the TTR promoter sequence comprises SEQ ID NO:26, optionally the TTR promoter sequence consists of SEQ ID NO:26.

Embodiment 6—An isolated polynucleotide comprising from 5′ to 3′: (a) an hHCR1 sequence that is at least 85% identical to SEQ ID NO:22, and a TTR promoter sequence that is at least 85% identical to SEQ ID NO:26; (b) a reverse complement hHCR1 sequence that is at least 85% identical to SEQ ID NO:23, and a TTR promoter sequence that is at least 85% identical to SEQ ID NO:26; (c) an hHCR1 sequence that is at least 85% identical to SEQ ID NO:24, and a TTR promoter sequence that is at least 85% identical to SEQ ID NO:26; or (d) a reverse complement hHCR1 sequence that is at least 85% identical to SEQ ID NO:25, and a TTR promoter sequence that is at least 85% identical to SEQ ID NO:26.

Embodiment 7—The isolated polynucleotide of embodiment 6, comprising from 5′ to 3′: (a) an hHCR1 sequence consisting of SEQ ID NO:22, and a TTR promoter sequence consisting of SEQ ID NO:26; (b) a reverse complement hHCR1 sequence consisting of SEQ ID NO:23, and a TTR promoter sequence consisting of SEQ ID NO:26; (c) an hHCR1 sequence consisting of SEQ ID NO:24, and a TTR promoter sequence consisting of SEQ ID NO:26; or (d) a reverse complement hHCR1 sequence consisting of SEQ ID NO:25, and a TTR promoter sequence consisting of SEQ ID NO:26.

Embodiment 8—The isolated polynucleotide of embodiment 6, comprising a sequence selected from the group consisting of SEQ ID NOs:41-43.

Embodiment 9—The isolated polynucleotide of any one of embodiments 4-7, further comprising a polyadenylation sequence, or a reverse complement thereof, interposed between the hHCR1 sequence or the reverse complement hHCR1 sequence and the TTR promoter sequence.

Embodiment 10—The isolated polynucleotide of embodiment 9, wherein the polyadenylation sequence is a beta globin polyadenylation (BGpA) sequence.

Embodiment 11—The isolated polynucleotide of embodiment 10, wherein the BGpA sequence comprises a sequence that is at least 85% identical to SEQ ID NO:27, optionally wherein the BGpA sequence comprises SEQ ID NO:27, optionally wherein the BGpA sequence consists of SEQ ID NO:27.

Embodiment 12—The isolated polynucleotide of any one of embodiments 4-11, comprising a sequence that is at least 85% identical to a sequence selected from the group consisting of SEQ ID NOs:60-71 and 76-78.

Embodiment 13—The isolated polynucleotide of embodiment 12, comprising to a sequence selected from the group consisting of SEQ ID NOs:60-71 and 76-78.

Embodiment 14—The isolated polynucleotide of embodiment any one of the preceding embodiments, further comprising a first intron element positioned 5′ to the reverse complement hAAT promoter sequence.

Embodiment 15—The isolated polynucleotide of embodiment any one of embodiments 4-14, further comprising a second intron element positioned 3′ to the TTR promoter sequence.

Embodiment 16—The isolated polynucleotide of embodiment 14 or 15, wherein the first and/or second intron element is a chimeric intron element.

Embodiment 17—The isolated polynucleotide of any one of embodiments 14-16, wherein the first intron element comprises a sequence that is at least 85% identical to SEQ ID NO:28, 29, 30, 31, or 32, optionally wherein the first intron element comprises SEQ ID NO:28, 29, 30, 31, or 32, optionally wherein the sequence of the first intron element consists of SEQ ID NO:28, 29, 30, 31, or 32.

Embodiment 18—The isolated polynucleotide of any one of embodiments 15-17, wherein the second intron element comprises a sequence that is at least 85% identical to SEQ ID NO:28, 29, 30, 31, or 32, optionally wherein the second intron element comprises SEQ ID NO:28, 29, 30, 31, or 32, optionally wherein the sequence of the second intron element consists of SEQ ID NO:28, 29, 30, 31, or 32.

Embodiment 19—The isolated polynucleotide of any one of embodiments 15-18, comprising from 5′ to 3′: (a) a first intron element comprising a sequence that is at least 85% identical to SEQ ID NO:28; a nucleic sequence selected from the group consisting of SEQ ID NO:60-71 and 76-78; and a second intron element comprising a sequence that is at least 85% identical to SEQ ID NO:29; (b) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:30; (c) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:31; or (d) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:32.

Embodiment 20—The isolated polynucleotide of any one of embodiments 15-19, comprising from 5′ to 3′: (a) a first intron element comprising the sequence of SEQ ID NO:28; a nucleic sequence selected from the group consisting of SEQ ID NOs:60-71 and 76-78; and a second intron element comprising the sequence of SEQ ID NO:29; (b) a first intron element comprising the sequence of SEQ ID NO:28; a nucleic sequence selected from the group consisting of SEQ ID NOs:60-71 and 76-78; and a second intron element comprising the sequence of SEQ ID NO:30; (c) a first intron element comprising the sequence of SEQ ID NO:28; a nucleic sequence selected from the group consisting of SEQ ID NOs:60-71 and 76-78; and a second intron element comprising the sequence of SEQ ID NO:31; or (d) a first intron element comprising the sequence of SEQ ID NO:28; a nucleic sequence selected from the group consisting of SEQ ID NOs:60-71 and 76-78; and a second intron element comprising the sequence of SEQ ID NO:32.

Embodiment 21—The isolated polynucleotide of any one of embodiments 4-20, comprising a sequence that is at least 85% identical to a sequence selected from the group consisting of SEQ ID NOs:79-116.

Embodiment 22—The isolated polynucleotide of embodiment 21, comprising to a sequence selected from the group consisting of SEQ ID NOs:79-116.

Embodiment 23—The isolated polynucleotide of any one of embodiments 4-22, further comprising: a reverse complement first coding sequence positioned 5′ of the reverse complement hAAT promoter sequence or the first intron element; and a second coding sequence positioned 3′ of the TTR promoter sequence of the second intron element.

Embodiment 24—The isolated polynucleotide of embodiment 23, further comprising a reverse complement first polyadenylation sequence positioned 5′ to the reverse complement first coding sequence.

Embodiment 25—The isolated polynucleotide of embodiment 24, wherein the reverse complement first polyadenylation sequence comprises a sequence that is at least 85% identical to SEQ ID NO:33, 34, or 35, optionally wherein the reverse complement first polyadenylation sequence comprises SEQ ID NO:33, 34, or 35, optionally wherein the sequence of the reverse complement first polyadenylation sequence consists of SEQ ID NO:33, 34, or 35.

Embodiment 26—The isolated polynucleotide of any one of embodiments 23-25, further comprising a second polyadenylation sequence positioned 3′ to the second coding sequence.

Embodiment 27—The isolated polynucleotide of embodiment 26, wherein the second polyadenylation sequence comprises a sequence that is at least 85% identical to SEQ ID NO:33, 34, or 35, optionally wherein the second polyadenylation sequence comprises SEQ ID NO:33, 34, or 35, optionally wherein the sequence of the second polyadenylation sequence consists of SEQ ID NO:33, 34, or 35.

Embodiment 28—The isolated polynucleotide of embodiment 27, comprising from 5′ to 3′: (a) a reverse complement first polyadenylation sequence comprising a sequence that is at least 85% identical to SEQ ID NO:33; a reverse complement first coding sequence; a nucleic sequence that is at least 85% identical to a nucleic acid sequence selected from the group consisting of SEQ ID NOs:60-71 and 76-116; a second coding sequence; and a second polyadenylation sequence comprising a sequence that is at least 85% identical to SEQ ID NO:35; or (b) a reverse complement first polyadenylation sequence comprising a sequence that is at least 85% identical to SEQ ID NO:34; a reverse complement first coding sequence; a nucleic sequence that is at least 85% identical to a nucleic acid sequence selected from the group consisting of SEQ ID NOs:60-71 and 76-116; a second coding sequence; and a second polyadenylation sequence comprising a sequence that is at least 85% identical to SEQ ID NO:35.

Embodiment 29—The isolated polynucleotide of embodiment 27, comprising from 5′ to 3′: (a) a reverse complement first polyadenylation sequence comprising the sequence of SEQ ID NO:33; a reverse complement first coding sequence; a nucleic sequence selected from the group consisting of SEQ ID NOs:60-71 and 76-116; a second coding sequence; and a second polyadenylation sequence comprising the sequence of SEQ ID NO:35; or (b) a reverse complement first polyadenylation sequence comprising the sequence of SEQ ID NO:34; a reverse complement first coding sequence; a nucleic sequence selected from the group consisting of SEQ ID NOs:60-71 and 76-116; a second coding sequence; and a second polyadenylation sequence comprising the sequence of SEQ ID NO:35.

Embodiment 30—The isolated polynucleotide of any one of embodiments 23-29, wherein: (a) the first coding sequence encodes an antibody heavy chain or an antigen-binding fragment thereof, and the second coding sequence encodes an antibody light chain or an antigen-binding fragment thereof or (b) the first coding sequence encodes an antibody light chain or an antigen-binding fragment thereof and the second coding sequence encodes an antibody heavy chain or an antigen-binding fragment thereof.

Embodiment 31—The isolated polynucleotide of embodiment 30, wherein the antibody heavy chain comprises the amino acid sequence of SEQ ID NO:178, and the antibody light chain comprises the amino acid sequence of SEQ ID NO:179.

Embodiment 32—The isolated polynucleotide of embodiment 30, wherein: (a) the first coding sequence comprises a nucleic acid sequence selected from the group consisting of SEQ ID NOs:178-199, and the second coding sequence comprises a nucleic acid sequence selected from the group consisting of SEQ ID NOs:200-217; or (b) the first coding sequence comprises a nucleic acid sequence selected from the group consisting of SEQ ID NOs:200-217, and the second coding sequence comprises a nucleic acid sequence selected from the group consisting of SEQ ID NOs:178-199.

Embodiment 33—The isolated polynucleotide of embodiment 30, wherein: (a) the first coding sequence and the second coding sequence, respectively, comprise the nucleic acid sequences set forth in SEQ ID NOs:200 and 181, 191 and 209, 193 and 211, 195 and 213, 197 and 215, or 199 and 217; or (b) the first coding sequence and the second coding sequence, respectively, comprise the nucleic acid sequences set forth in SEQ ID NOs:181 and 200, 209 and 191, 211 and 193, 213 and 195, 215 and 197, or 217 and 199.

Embodiment 34—The isolated polynucleotide of any one of embodiments 30-33, wherein the first coding sequence and/or the second coding sequence comprises a signal sequence, optionally wherein the signal sequence comprises the amino acid sequence of SEQ ID NO:167 or 173.

Embodiment 35—The isolated polynucleotide of any one of embodiments 30-33, wherein the first coding sequence and/or the second coding sequence comprise a nucleic acid sequence selected from the group consisting of SEQ ID NOs:168-172 and 174-177.

Embodiment 36—The isolated polynucleotide of embodiment 35, comprising from 5′ to 3′: (a) SEQ ID NO:33; the reverse complement of SEQ ID NO:191; the reverse complement of SEQ ID NO:172, a nucleic sequence selected from the group consisting of SEQ ID NOs:60-71 and 76-116; SEQ ID NO:177; SEQ ID NO:209; and SEQ ID NO:35; (b) SEQ ID NO:34; the reverse complement of SEQ ID NO:181; the reverse complement of SEQ ID NO:168, a nucleic sequence selected from the group consisting of SEQ ID N060-71 and 76-116; SEQ ID NO:174; SEQ ID NO:200; and SEQ ID NO:35; (c) SEQ ID NO:34; the reverse complement of SEQ ID NO:191; the reverse complement of SEQ ID NO:172, a nucleic sequence selected from the group consisting of SEQ ID NOs:60-71 and 76-116; SEQ ID NO:177; SEQ ID NO:209; and SEQ ID NO:35; (d) SEQ ID NO:34; the reverse complement of SEQ ID NO:193; the reverse complement of SEQ ID NO:172, a nucleic sequence selected from the group consisting of SEQ ID NOs:60-71 and 76-116; SEQ ID NO:177; SEQ ID NO:211; and SEQ ID NO:35; (e) SEQ ID NO:34; the reverse complement of SEQ ID NO:195; the reverse complement of SEQ ID NO:172, a nucleic sequence selected from the group consisting of SEQ ID NOs:60-71 and 76-116; SEQ ID NO:177; SEQ ID NO:213; and SEQ ID NO:35; (f) SEQ ID NO:34; the reverse complement of SEQ ID NO:197; the reverse complement of SEQ ID NO:172, a nucleic sequence selected from the group consisting of SEQ ID NOs:60-71 and 76-116; SEQ ID NO:177; SEQ ID NO:215; and SEQ ID NO:35; or (g) SEQ ID NO:34; the reverse complement of SEQ ID NO:199; the reverse complement of SEQ ID NO:172, a nucleic sequence selected from the group consisting of SEQ ID NOs:60-71 and 76-116; SEQ ID NO:177; SEQ ID NO:217; and SEQ ID NO:35.

Embodiment 37—The isolated polynucleotide of embodiment 1, comprising from 5′ to 3′: (a) SEQ ID NO:33; the reverse complement of SEQ ID NO:209; the reverse complement of SEQ ID NO:177, a nucleic sequence selected from the group consisting of SEQ ID NOs:60-71 and 76-116; SEQ ID NO:172; SEQ ID NO:191; and SEQ ID NO:35; (b) SEQ ID NO:34; the reverse complement of SEQ ID NO:200; the reverse complement of SEQ ID NO:174, a nucleic sequence selected from the group consisting of SEQ ID NOs:60-71 and 76-116; SEQ ID NO:168; SEQ ID NO:181; and SEQ ID NO:35; (c) SEQ ID NO:34; the reverse complement of SEQ ID NO:209; the reverse complement of SEQ ID NO:177, a nucleic sequence selected from the group consisting of SEQ ID NOs:60-71 and 76-116; SEQ ID NO:172; SEQ ID NO:191; and SEQ ID NO:35; (d) SEQ ID NO:34; the reverse complement of SEQ ID NO:211; the reverse complement of SEQ ID NO:177, a nucleic sequence selected from the group consisting of SEQ ID NOs:60-71 and 76-116; SEQ ID NO:172; SEQ ID NO:193; and SEQ ID NO:35; (e) SEQ ID NO:34; the reverse complement of SEQ ID NO:213; the reverse complement of SEQ ID NO:177, a nucleic sequence selected from the group consisting of SEQ ID NOs:60-71 and 76-116; SEQ ID NO:172; SEQ ID NO:195; and SEQ ID NO:35; (f) SEQ ID NO:34; the reverse complement of SEQ ID NO:215; the reverse complement of SEQ ID NO:177, a nucleic sequence selected from the group consisting of SEQ ID NOs:60-71 and 76-116; SEQ ID NO:172; SEQ ID NO:197; and SEQ ID NO:35; or (g) SEQ ID NO:34; the reverse complement of SEQ ID NO:217; the reverse complement of SEQ ID NO:177, a nucleic sequence selected from the group consisting of SEQ ID NOs:60-71 and 76-116; SEQ ID NO:172; SEQ ID NO:199; and SEQ ID NO:35.

Embodiment 38—The isolated polynucleotide of any one of embodiments 1-3, further comprising a polyadenylation sequence, or a reverse complement thereof, positioned 3′ of the hHCR1 sequence or the reverse complement hHCR1 sequence.

Embodiment 39—The isolated polynucleotide of embodiment 38, wherein the polyadenylation sequence is a beta globin polyadenylation (BGpA) sequence, optionally wherein the BGpA sequence comprises a sequence that is at least 85% identical to SEQ ID NO:27, optionally wherein the BGpA sequence comprises SEQ ID NO:27, optionally wherein the BGpA sequence consists of SEQ ID NO:27.

Embodiment 40—An isolated polynucleotide comprising from 5′ to 3′: a polyadenylation sequence, or a reverse complement thereof; and a TTR promoter sequence.

Embodiment 41—The isolated polynucleotide of embodiment 40, wherein the TTR promoter sequence comprises a nucleic acid sequence that is at least 85% identical to SEQ ID NO:26, optionally the TTR promoter sequence comprises SEQ ID NO:26, optionally the TTR promoter sequence consists of SEQ ID NO:26.

Embodiment 42—The isolated polynucleotide of embodiment 40 or 41, wherein the polyadenylation sequence is a beta globin polyadenylation (BGpA) sequence, optionally wherein the BGpA sequence comprises a sequence that is at least 85% identical to SEQ ID NO:27, optionally wherein the BGpA sequence comprises SEQ ID NO:27, optionally wherein the BGpA sequence consists of SEQ ID NO:27.

Embodiment 43—An isolated polynucleotide, comprising: (a) a nucleic acid sequence selected from the group consisting of SEQ ID NOs:19, 29-31, 44-59, 117-140, 168-172, 174-177 and 180-217; (b) an hAAT promoter sequence consisting of a sequence that is at least 85% identical to SEQ ID NO:21, or 22, optionally wherein the hAAT promoter sequence consists of SEQ ID NO:21, or 22; or (c) an hAAT promoter sequence comprising SEQ ID NO:19, optionally wherein the hAAT promoter sequence consists of SEQ ID NO:19.

Embodiment 44—A polynucleotide that is the complement of the polynucleotide of any one of the preceding embodiments.

Embodiment 45—A polynucleotide that is the reverse complement of the polynucleotide of any one of the preceding embodiments.

Embodiment 46—A recombinant adeno-associated virus (rAAV) genome comprising a polynucleotide of any the preceding embodiments.

Embodiment 47—The rAAV genome of embodiment 46, wherein the rAAV genome further comprises a 5′ inverted terminal repeat (5′ ITR) sequence, and a 3′ inverted terminal repeat (3′ ITR) sequence.

Embodiment 48—The rAAV genome of embodiment 47, wherein the 5′ ITR nucleic acid sequence is at least 85% identical to the nucleic acid sequence set forth in SEQ ID NO:165, and/or the 3′ ITR nucleic acid sequence is at least 85% identical to the nucleic acid sequence set forth in SEQ ID NO:166.

Embodiment 49—The rAAV genome of embodiment 48, wherein the 5′ ITR nucleic acid sequence comprises the nucleic acid sequence set forth in SEQ ID NO:165, and/or the 3′ ITR nucleic acid sequence comprises the nucleic acid sequence set forth in SEQ ID NO:166.

Embodiment 50—The rAAV genome of embodiment 46, wherein the rAAV genome comprises a nucleic acid sequence that is at least 85% to the nucleic acid sequence set forth in SEQ ID NOs:141-164, or the complement thereof.

Embodiment 51—The rAAV genome of embodiment 46, wherein the rAAV genome comprises the nucleic acid sequence set forth in SEQ ID NOs:141-164, or the complement thereof.

Embodiment 52—The rAAV genome of any one of embodiments 46-51, wherein the rAAV genome is a single stranded rAAV genome.

Embodiment 53—The rAAV genome of any one of embodiments 46-51, wherein the rAAV genome is a self-complementary rAAV genome.

Embodiment 54—A nucleic acid vector comprising the isolated polynucleotide or rAAV genome of any one of the preceding embodiments, optionally wherein the nucleic acid vector is a plasmid, a virus, or a DNA minimal vector.

Embodiment 55—An rAAV comprising: an AAV capsid comprising an AAV capsid protein; and an rAAV genome of any one of the embodiments 46-53.

Embodiment 56—The rAAV of embodiment 55, wherein the capsid protein is selected from the group consisting of AAV1, AAV2, AAV3, AAV4, AAV5, AAV6, AAV7, AAV8, AAV9, AAVrh10, AAVrh74, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PHP.S.

Embodiment 57—The rAAV of embodiment 55 or 56, wherein the AAV capsid protein comprises an amino acid sequence that is at least 95% identical to the amino acid sequence of amino acids 203-736 of any one of SEQ ID NOs:1-15.

Embodiment 58—The rAAV of embodiment 57, wherein: the amino acid in the capsid protein corresponding to amino acid 206 of SEQ ID NO:15 is C; the amino acid in the capsid protein corresponding to amino acid 296 of SEQ ID NO:15 is H; the amino acid in the capsid protein corresponding to amino acid 312 of SEQ ID NO:15 is Q; the amino acid in the capsid protein corresponding to amino acid 346 of SEQ ID NO:15 is A; the amino acid in the capsid protein corresponding to amino acid 464 of SEQ ID NO:15 is N; the amino acid in the capsid protein corresponding to amino acid 468 of SEQ ID NO:15 is S; the amino acid in the capsid protein corresponding to amino acid 501 of SEQ ID NO:15 is I; the amino acid in the capsid protein corresponding to amino acid 505 of SEQ ID NO:15 is R; the amino acid in the capsid protein corresponding to amino acid 590 of SEQ ID NO:15 is R; the amino acid in the capsid protein corresponding to amino acid 626 of SEQ ID NO:15 is G or Y; the amino acid in the capsid protein corresponding to amino acid 681 of SEQ ID NO:15 is M; the amino acid in the capsid protein corresponding to amino acid 687 of SEQ ID NO:15 is R; the amino acid in the capsid protein corresponding to amino acid 690 of SEQ ID NO:15 is K; the amino acid in the capsid protein corresponding to amino acid 706 of SEQ ID NO:15 is C; or, the amino acid in the capsid protein corresponding to amino acid 718 of SEQ ID NO:15 is G.

Embodiment 59—The rAAV of embodiment 57, wherein: (a) the amino acid in the capsid protein corresponding to amino acid 626 of SEQ ID NO:15 is G, and the amino acid in the capsid protein corresponding to amino acid 718 of SEQ ID NO:15 is G; (b) the amino acid in the capsid protein corresponding to amino acid 296 of SEQ ID NO:15 is H, the amino acid in the capsid protein corresponding to amino acid 464 of SEQ ID NO:15 is N, the amino acid in the capsid protein corresponding to amino acid 505 of SEQ ID NO:15 is R, and the amino acid in the capsid protein corresponding to amino acid 681 of SEQ ID NO:15 is M; (c) the amino acid in the capsid protein corresponding to amino acid 505 of SEQ ID NO:15 is R, and the amino acid in the capsid protein corresponding to amino acid 687 of SEQ ID NO:15 is R; (d) the amino acid in the capsid protein corresponding to amino acid 346 of SEQ ID NO:15 is A, and the amino acid in the capsid protein corresponding to amino acid 505 of SEQ ID NO:15 is R; or (e) the amino acid in the capsid protein corresponding to amino acid 501 of SEQ ID NO:15 is I, the amino acid in the capsid protein corresponding to amino acid 505 of SEQ ID NO:15 is R, and the amino acid in the capsid protein corresponding to amino acid 706 of SEQ ID NO:15 is C.

Embodiment 60—The rAAV of embodiment 57, wherein the capsid protein comprises the amino acid sequence of amino acids 203-736 of any one of SEQ ID NOs:1-15.

Embodiment 61—The rAAV of embodiment 60, wherein the AAV capsid protein is encoded by the nucleic acid sequence of nucleotides 607-2208 of SEQ ID NO:16.

Embodiment 62—The rAAV of embodiment 55 or 56, wherein the AAV capsid protein comprises an amino acid sequence that is at least 95% identical to the amino acid sequence of amino acids 138-736 of any one of SEQ ID NOs:1-15.

Embodiment 63—The rAAV of embodiment 62, wherein: the amino acid in the capsid protein corresponding to amino acid 151 of SEQ ID NO:15 is R; the amino acid in the capsid protein corresponding to amino acid 160 of SEQ ID NO:15 is D; the amino acid in the capsid protein corresponding to amino acid 206 of SEQ ID NO:15 is C; the amino acid in the capsid protein corresponding to amino acid 296 of SEQ ID NO:15 is H; the amino acid in the capsid protein corresponding to amino acid 312 of SEQ ID NO:15 is Q; the amino acid in the capsid protein corresponding to amino acid 346 of SEQ ID NO:15 is A; the amino acid in the capsid protein corresponding to amino acid 464 of SEQ ID NO:15 is N; the amino acid in the capsid protein corresponding to amino acid 468 of SEQ ID NO:15 is S; the amino acid in the capsid protein corresponding to amino acid 501 of SEQ ID NO:15 is I; the amino acid in the capsid protein corresponding to amino acid 505 of SEQ ID NO:15 is R; the amino acid in the capsid protein corresponding to amino acid 590 of SEQ ID NO:15 is R; the amino acid in the capsid protein corresponding to amino acid 626 of SEQ ID NO:15 is G or Y; the amino acid in the capsid protein corresponding to amino acid 681 of SEQ ID NO:15 is M; the amino acid in the capsid protein corresponding to amino acid 687 of SEQ ID NO:15 is R; the amino acid in the capsid protein corresponding to amino acid 690 of SEQ ID NO:15 is K; the amino acid in the capsid protein corresponding to amino acid 706 of SEQ ID NO:15 is C; or, the amino acid in the capsid protein corresponding to amino acid 718 of SEQ ID NO:15 is G.

Embodiment 64—The rAAV of embodiment 63, wherein: (a) the amino acid in the capsid protein corresponding to amino acid 626 of SEQ ID NO:15 is G, and the amino acid in the capsid protein corresponding to amino acid 718 of SEQ ID NO:15 is G; (b) the amino acid in the capsid protein corresponding to amino acid 296 of SEQ ID NO:15 is H, the amino acid in the capsid protein corresponding to amino acid 464 of SEQ ID NO:15 is N, the amino acid in the capsid protein corresponding to amino acid 505 of SEQ ID NO:15 is R, and the amino acid in the capsid protein corresponding to amino acid 681 of SEQ ID NO:15 is M; (c) the amino acid in the capsid protein corresponding to amino acid 505 of SEQ ID NO:15 is R, and the amino acid in the capsid protein corresponding to amino acid 687 of SEQ ID NO:15 is R; (d) the amino acid in the capsid protein corresponding to amino acid 346 of SEQ ID NO:15 is A, and the amino acid in the capsid protein corresponding to amino acid 505 of SEQ ID NO:15 is R; or (e) the amino acid in the capsid protein corresponding to amino acid 501 of SEQ ID NO:15 is I, the amino acid in the capsid protein corresponding to amino acid 505 of SEQ ID NO:15 is R, and the amino acid in the capsid protein corresponding to amino acid 706 of SEQ ID NO:15 is C.

Embodiment 65—The rAAV of embodiment 63, wherein the capsid protein comprises the amino acid sequence of amino acids 138-736 of any one of SEQ ID NOs:1-15.

Embodiment 66—The rAAV of embodiment 63, wherein the AAV capsid protein is encoded by the nucleic acid sequence of nucleotides 414-2208 of SEQ ID NO:16.

Embodiment 67—The rAAV of embodiment 55 or 56, wherein the AAV capsid protein comprises an amino acid sequence that is at least 95% identical to the amino acid sequence of amino acids 1-736 of any one of SEQ ID NOs:1-15.

Embodiment 68—The rAAV of embodiment 67, wherein: the amino acid in the capsid protein corresponding to amino acid 2 of SEQ ID NO:15 is T; the amino acid in the capsid protein corresponding to amino acid 65 of SEQ ID NO:15 is I; the amino acid in the capsid protein corresponding to amino acid 68 of SEQ ID NO:15 is V; the amino acid in the capsid protein corresponding to amino acid 77 of SEQ ID NO:15 is R; the amino acid in the capsid protein corresponding to amino acid 119 of SEQ ID NO:15 is L; the amino acid in the capsid protein corresponding to amino acid 151 of SEQ ID NO:15 is R; the amino acid in the capsid protein corresponding to amino acid 160 of SEQ ID NO:15 is D; the amino acid in the capsid protein corresponding to amino acid 206 of SEQ ID NO:15 is C; the amino acid in the capsid protein corresponding to amino acid 296 of SEQ ID NO:15 is H; the amino acid in the capsid protein corresponding to amino acid 312 of SEQ ID NO:15 is Q; the amino acid in the capsid protein corresponding to amino acid 346 of SEQ ID NO:15 is A; the amino acid in the capsid protein corresponding to amino acid 464 of SEQ ID NO:15 is N; the amino acid in the capsid protein corresponding to amino acid 468 of SEQ ID NO:15 is S; the amino acid in the capsid protein corresponding to amino acid 501 of SEQ ID NO:15 is I; the amino acid in the capsid protein corresponding to amino acid 505 of SEQ ID NO:15 is R; the amino acid in the capsid protein corresponding to amino acid 590 of SEQ ID NO:15 is R; the amino acid in the capsid protein corresponding to amino acid 626 of SEQ ID NO:15 is G or Y; the amino acid in the capsid protein corresponding to amino acid 681 of SEQ ID NO:15 is M; the amino acid in the capsid protein corresponding to amino acid 687 of SEQ ID NO:15 is R; the amino acid in the capsid protein corresponding to amino acid 690 of SEQ ID NO:15 is K; the amino acid in the capsid protein corresponding to amino acid 706 of SEQ ID NO:15 is C; or, the amino acid in the capsid protein corresponding to amino acid 718 of SEQ ID NO:15 is G.

Embodiment 69—The rAAV of embodiment 68, wherein: (a) the amino acid in the capsid protein corresponding to amino acid 2 of SEQ ID NO:15 is T, and the amino acid in the capsid protein corresponding to amino acid 312 of SEQ ID NO:15 is Q; (b) the amino acid in the capsid protein corresponding to amino acid 65 of SEQ ID NO:15 is I, and the amino acid in the capsid protein corresponding to amino acid 626 of SEQ ID NO:15 is Y; (c) the amino acid in the capsid protein corresponding to amino acid 77 of SEQ ID NO:15 is R, and the amino acid in the capsid protein corresponding to amino acid 690 of SEQ ID NO:15 is K; (d) the amino acid in the capsid protein corresponding to amino acid 119 of SEQ ID NO:15 is L, and the amino acid in the capsid protein corresponding to amino acid 468 of SEQ ID NO:15 is S; (e) the amino acid in the capsid protein corresponding to amino acid 626 of SEQ ID NO:15 is G, and the amino acid in the capsid protein corresponding to amino acid 718 of SEQ ID NO:15 is G; (f) the amino acid in the capsid protein corresponding to amino acid 296 of SEQ ID NO:15 is H, the amino acid in the capsid protein corresponding to amino acid 464 of SEQ ID NO:15 is N, the amino acid in the capsid protein corresponding to amino acid 505 of SEQ ID NO:15 is R, and the amino acid in the capsid protein corresponding to amino acid 681 of SEQ ID NO:15 is M; (g) the amino acid in the capsid protein corresponding to amino acid 505 of SEQ ID NO:15 is R, and the amino acid in the capsid protein corresponding to amino acid 687 of SEQ ID NO:15 is R; (h) the amino acid in the capsid protein corresponding to amino acid 346 of SEQ ID NO:15 is A, and the amino acid in the capsid protein corresponding to amino acid 505 of SEQ ID NO:15 is R; or (i) the amino acid in the capsid protein corresponding to amino acid 501 of SEQ ID NO:15 is I, the amino acid in the capsid protein corresponding to amino acid 505 of SEQ ID NO:15 is R, and the amino acid in the capsid protein corresponding to amino acid 706 of SEQ ID NO:15 is C.

Embodiment 70—The rAAV of embodiment 68, wherein the capsid protein comprises the amino acid sequence of amino acids 1-736 of SEQ ID NOs:1-15.

Embodiment 71—The rAAV of embodiment 70, wherein the AAV capsid protein is encoded by the nucleic acid sequence set forth in SEQ ID NO:16.

Embodiment 72—A pharmaceutical composition comprising a polynucleotide of any one of embodiments 1-45, an rAAV genome of any one of embodiments 46-54, or an rAAV of any one of embodiments 55-71.

Embodiment 73—A packaging system for preparation of an rAAV, wherein the packaging system comprises: (a) a first nucleic acid sequence encoding one or more AAV Rep proteins; (b) a second nucleic acid sequence encoding a capsid protein of the rAAV of any one of embodiments 55-71; and (c) a third nucleic acid sequence comprising an rAAV genome sequence of the rAAV of any one of embodiments 46-53.

Embodiment 74—The packaging system of embodiment 73, wherein the packaging system comprises a first vector comprising the first nucleic acid sequence and the second nucleic acid sequence, and a second vector comprising the third nucleic acid sequence.

Embodiment 75—The packaging system of embodiment 73 or 74, further comprising a fourth nucleic acid sequence comprising one or more helper virus genes.

Embodiment 76—The packaging system of embodiment 75, wherein the fourth nucleic acid sequence is comprised within a third vector.

Embodiment 77—The packaging system of embodiment 75 or 76, wherein the fourth nucleic acid sequence comprises one or more genes from a virus selected from the group consisting of adenovirus, herpes virus, vaccinia virus, and cytomegalovirus (CMV).

Embodiment 78—The packaging system of any one of embodiments 73-77, wherein the first vector, second vector, and/or the third vector is a plasmid or a DNA minimal vector.

Embodiment 79—A method for recombinant preparation of an rAAV, the method comprising introducing the packaging system of any one of embodiments 73-78 into a cell under conditions whereby the rAAV is produced.

Embodiment 80—The rAAV of any one of embodiments 55-71, the polynucleotide of any one of embodiments 1-45, the rAAV genome of any one of embodiments 46-54, or the pharmaceutical composition of embodiment 72, for use as a medicament.

Embodiment 81—The rAAV of any one of embodiments 55-71, the polynucleotide of any one of embodiments 1-45, the rAAV genome of any one of embodiments 46-54, or the pharmaceutical composition of embodiment 72, for use in the treatment of a complement C5-associated disease.

Embodiment 82—The rAAV of any one of embodiments 55-71, the polynucleotide of any one of embodiments 1-45, the rAAV genome of any one of embodiments 46-54, or the pharmaceutical composition of embodiment 72, for use in a method of treating a subject having a complement C5-associated disease, the method comprising administering to the subject an effective amount of the rAAV, the pharmaceutical composition, or the polynucleotide.

Embodiment 83—A method of producing an antibody in a subject, the method comprising administering to the subject the rAAV of any one of embodiments 55-71, the polynucleotide of any one of embodiments 1-45, the rAAV genome of any one of embodiments 46-54, or the pharmaceutical composition of embodiment 72.

Embodiment 84—A method of treating a complement C5-associated disease in a subject in need thereof, the method comprising administering to the subject an effective amount of the rAAV of any one of embodiments 55-71, the polynucleotide of any one of embodiments 1-45, the rAAV genome of any one of embodiments 46-54, or the pharmaceutical composition of embodiment 72.

Embodiment 85—The method of embodiment 84, wherein the complement C5-associated disease is selected from the group consisting of geographic atrophy (GA), Guillain-Barré syndrome, myasthenia gravis, systemic lupus erythematous (SLE) nephritis, proliferative nephritis, asthma, rheumatoid arthritis, sepsis, paroxysmal nocturnal hemoglobinuria (PNH), atypical hemolytic uremic syndrome (aHUS), neuromyelitis optica spectrum disorders (NMOSD), and age-related macular degeneration (AMD).

Embodiment 86—The method of any one of embodiments 83-85, wherein the rAAV, the polynucleotide, the rAAV genome, or the pharmaceutical composition, is administered intravenously.

In any of the preceding embodiments and for all polynucleotides disclosed herein, individual sequence elements (e.g., hAAT promoter sequence, hHCR sequence, etc.) present in any of the polynucleotides can be contiguous or can have additional nucleotides interposed between the sequence elements. In certain embodiments, at least 2 (e.g., 2, 3, 4, 5, 6, 7, 8, 9, 10, 11, or 12) of the individual sequence elements are contiguous. In certain embodiments, all of the individual sequence elements are contiguous.

### BRIEF DESCRIPTION OF THE DRAWINGS

FIG. 1 is a graph showing anti-C5 antibody concentration in the serum of NOD-SCID mice administered a single 1e13 vg/kg dose of the indicated anti-C5 antibody-expressing rAAV vectors, in each case packaged in either an AAVHSC15 capsid or an AAVHSC17 capsid, or a buffer control. Ab-02 uses a single promoter to drive expression of the antibody heavy chain and light chain, Ab-03 uses two tandem promoters to drive expression of the antibody heavy chain and light chain, and Ab-04 uses a bidirectional dual promoter system to drive expression of the antibody heavy chain and light chain. Serum samples were collected at 1, 3, 5, 7, 9, 13, and 16 weeks (3 mice per time point), and antibody levels were measured using a human IgG-specific ELISA. Vectors Ab-02, Ab-03, and Ab-04 are described in detail in Example 1.

FIG. 2A and FIG. 2B are graphs showing anti-C5 antibody concentration in the serum of NOD-SCID mice administered a single 1e13 vg/kg dose of the indicated anti-C5 antibody-expressing rAAV vectors, in each case packaged in an AAVHSC13 capsid, or a buffer control (CTR). Serum samples were collected at 1, 3, 5, 7, 9, 11, and 12 weeks (5 mice per time point), and antibody levels were measured using a human IgG-specific ELISA. Details of the rAAV vectors used in these experiments are set forth in Table 1.

FIG. 3 is a graph showing the number of vector genomes per μg of genomic DNA in the liver of NOD-SCID mice 12 weeks after administration of a single 1e13 vg/kg dose of the indicated anti-C5 antibody-expressing rAAV vectors, in each case packaged in an AAVHSC13 capsid, or a buffer control (FB). The number of vector genomes was determined by droplet digital PCR (ddPCR) using a primer probe set common to all vectors and not present in the murine genome. Details of the rAAV vectors used in these experiments are set forth in Table 1.

FIG. 4 is a graph showing the results of an ex vivo hemolysis assay testing the ability of serum from serum of NOD-SCID mice administered a single 1e13 vg/kg dose of the indicated anti-C5 antibody-expressing rAAV vectors (in each case packaged in an AAVHSC13 capsid) or a buffer control (FB) to inhibit C5-mediated hemolysis of sheep erythrocytes. Ex vivo hemolysis assays were performed using serum samples collected at 1, 3, and 12 weeks.

FIG. 5 is a graph showing anti-C5 antibody concentration in the serum of NOD-SCID mice administered a single 1e13 vg/kg dose of the indicated anti-C5 antibody-expressing rAAV vectors, in each case packaged in an AAVHSC13 capsid, or a buffer control. Serum samples were collected at 1, 3, 5, 7, 9, 11, and 12 weeks (5 mice per time point), and antibody levels were measured using a human IgG-specific ELISA. Details of the rAAV vectors used in these experiments are set forth in Table 1.

FIG. 6 is a graph showing anti-C5 antibody concentration in the serum of NOD-SCID mice administered a single 1e13 vg/kg dose of the indicated anti-C5 antibody-expressing rAAV vectors, in each case packaged in an AAVHSC13 capsid, or a buffer control. Serum samples were collected at 1, 3, 5, 7, 9, 11, and 12 weeks (5 mice per time point), and antibody levels were measured using a human IgG-specific ELISA. Details of the rAAV vectors used in these experiments are set forth in Table 1.

FIG. 7 is a graph showing anti-C5 antibody concentration in the serum of NOD-SCID mice administered a single 1e13 vg/kg dose of the indicated anti-C5 antibody-expressing rAAV vectors, in each case packaged in an AAVHSC13 capsid, or a buffer control. Serum samples were collected at 1, 3, 5, 7, 9, 11, and 12 weeks (5 mice per time point), and antibody levels were measured using a human IgG-specific ELISA. Details of the rAAV vectors used in these experiments are set forth in Table 1.

FIG. 8 is a graph showing anti-C5 antibody concentration in the serum of NOD-SCID mice administered a single 1e13 vg/kg dose of the indicated anti-C5 antibody-expressing rAAV vectors, in each case packaged in an AAVHSC13 capsid. Serum samples were collected at 1, 3, 5, 7, 9, 11, and 12 weeks (5 mice per time point), and antibody levels were measured using a human IgG-specific ELISA. Details of the rAAV vectors used in these experiments are set forth in Table 1.

FIG. 9A and FIG. 9B are graphs showing anti-C5 antibody concentration in the serum of NOD-SCID mice administered a single 1e13 vg/kg dose of the indicated anti-C5 antibody-expressing rAAV vectors, in each case packaged in an AAVHSC13 capsid. Serum samples were collected at 1, 3, 5, 7, 9, 11, and 12 weeks (5 mice per time point), and antibody levels were measured using a human IgG-specific ELISA. Details of the rAAV vectors used in these experiments are set forth in Table 1.

FIG. 10 is a graph showing anti-C5 antibody concentration in the serum of NOD-SCID mice administered a single 1e13 vg/kg dose of the indicated anti-C5 antibody-expressing rAAV vectors, in each case packaged in an AAVHSC13 capsid. Serum samples were collected at 1, 3, 5, 7, 9, 11, and 12 weeks (5 mice per time point), and antibody levels were measured using a human IgG-specific ELISA. Details of the rAAV vectors used in these experiments are set forth in Table 1.

FIG. 11A, FIG. 11B, and FIG. 11C are graphs showing anti-C5 antibody concentration in the serum of NOD-SCID mice administered a single 1e13 vg/kg dose of the indicated anti-C5 antibody-expressing rAAV vectors, in each case packaged in an AAVHSC13 capsid. Serum samples were collected at 1, 3, 5, 7, 9, 11, and 12 weeks (5 mice per time point), and antibody levels were measured using a human IgG-specific ELISA. Details of the rAAV vectors used in these experiments are set forth in Table 1.

FIG. 12A and FIG. 12B are graphs showing anti-C5 antibody concentration in the serum of NOD-SCID mice administered a single 1e13 vg/kg dose of the indicated anti-C5 antibody-expressing rAAV vectors, in each case packaged in an AAVHSC13 capsid. Serum samples were collected at 1, 3, 5, 7, 9, 11, and 12 weeks (5 mice per time point), and antibody levels were measured using a human IgG-specific ELISA. FIG. 12C is a graph showing anti-C5 antibody concentration in the serum of C57BL/6J mice administered a single 3e12 vg/kg dose of the indicated anti-C5 antibody-expressing rAAV vectors, in each case packaged in an AAVHSC13 capsid. Serum samples were collected at 1, 3, and 5 weeks (5 mice per time point), and antibody levels were measured using a human IgG-specific ELISA. Details of the rAAV vectors used in these experiments are set forth in Table 1.

FIG. 13A is a graph showing anti-C5 antibody concentration in the serum of NOD-SCID mice administered 3e11, 3e12, 1e13, 3e13, and 1e14 vg/kg of vector ES5 packaged in an AAVHSC13 capsid or formulation buffer control (vehicle). Serum samples were collected at 1, 3, 5, 7, 9, 11, 13, and 15 weeks (5 male mice per time point), and antibody levels were measured using a human IgG-specific ELISA. Terminal serum samples were collected from each mouse at 4 and 16 weeks. FIG. 13B is a graph showing the results of an ex vivo hemolysis assay testing the ability of serum from treated mice to inhibit C5-mediated hemolysis of sheep erythrocytes. Ex vivo hemolysis assays were performed using serum samples collected at 1, 3, 5, and 16 weeks. FIG. 13C is a graph showing the number of vector genomes per μg of genomic DNA in the liver of treated mice, as determined by droplet digital PCR (ddPCR) using a primer probe set common to the ES5 vector and not present in the murine genome. FIG. 13D and FIG. 13E are graphs showing the number of transcripts of codon optimized heavy chain (CO-HC) and codon optimized light chain (CO-LC), respectively, per μg of cDNA in treated animals, and FIG. 13F is a graph showing the ratio of heavy chain to light chain transcripts. In FIGS. 13C-13E, **** indicates statistical significance at p<0.0001, with statistical analysis performed on group means using a Mixed-effects model and Sidak's multiple comparison test. In FIG. 3F, statistical difference was observed between 4- and 16-week post dose for 3e12 vg/kg (**: p<0.01) and 1e14 vg/kg (****: p<0.0001), with statistical analysis performed on group means using a Mixed-effects model and Sidak's multiple comparison test.

FIG. 14A is a graph showing anti-C5 antibody concentration in the serum of HuLiv mice administered 3e12, 1e13, and 3e13 vg/kg of vector ES5 packaged in an AAVHSC13 capsid or formulation buffer control (vehicle). Serum samples were collected at 1 and 3 weeks (5 male mice per time point), and antibody levels were measured using a human IgG-specific ELISA. A terminal serum sample was collected at 4 weeks. FIG. 14B is a graph showing the results of an ex vivo hemolysis assay testing the ability of serum from treated mice to inhibit C5-mediated hemolysis of sheep erythrocytes. Ex vivo hemolysis assays were performed using serum samples collected at 1, 3, and 4 weeks. FIG. 14C is a graph showing the number of vector genomes per μg of genomic DNA in the liver of treated mice, as determined by droplet digital PCR (ddPCR) using a primer probe set common to the ES5 vector and not present in the murine genome. FIG. 14D is a graph showing the number of transcripts of codon optimized heavy chain (CO-HC) and codon optimized light chain (CO-LC) per μg of cDNA in treated animals, and FIG. 14E is a graph showing the ratio of heavy chain to light chain transcripts.

FIG. 15A is a graph showing anti-C5 antibody concentration in the serum of C57BL/6J mice treated with a single 1e13 vg/kg dose of vector E55 packaged in an AAVHSC13 capsid (AAVHSC13-E55), or with formulation buffer (vehicle) as control. Serum samples were collected from mice in the 4 week arm at 1 and 3 weeks with a terminal serum sample collected at 4 weeks, and serum samples collected from mice in the 26 week arm monthly with a terminal serum sample collected at 26 weeks. 13 male (M) and female (F) mice were dosed in each group. FIG. 15B is a graph showing the number of vector genomes per μg of genomic DNA in the liver of treated mice, as determined by droplet digital PCR (ddPCR), at 4 and 26 weeks post treatment. Statistical analysis was performed on group means using a one-way ANOVA and Sidak's multiple comparison test. ** indicates statistical significance at p=0.0014, and **** indicates statistical significance at p<0.0001.

### DETAILED DESCRIPTION

Provided herein are polynucleotides comprising novel bidirectional dual expression cassettes, recombination adeno-associated virus (rAAV) comprising these polynucleotides, and methods of making and using the polynucleotides and rAAV. Also provided are novel transcriptional control elements (e.g., promoters, enhancers, introns, polyadenylation sequences, and combinations thereof), and novel antibody coding sequences.

### Definitions

As used herein, the term “human α1-antitrypsin promoter sequence” or “hAAT promoter sequence” refers to an isolated transcriptional regulatory element comprising a nucleic acid sequence that is at least 75% identical to SEQ ID NO:21 and that exhibits promoter activity that is at least the same as the promoter activity of the nucleic acid sequence of SEQ ID NO:21, when compared under identical conditions.

As used herein, the term “human hepatic control region 1 sequence” or “hHCR1 sequence” refers to an isolated transcriptional regulatory element comprising a nucleic acid sequence that is at least 75% identical to SEQ ID NO:24 and that exhibits transcriptional enhancer activity that is at least the same as the promoter transcriptional enhancer activity of the nucleic acid sequence of SEQ ID NO:24, when compared under identical conditions.

As used herein, the term “transthyretin promoter sequence” or “TTR promoter sequence” refers to an isolated transcriptional regulatory element comprising a nucleic acid sequence that is at least 75% identical to SEQ ID NO:26 and that exhibits promoter activity that is at least the same as the promoter activity of the nucleic acid sequence of SEQ ID NO:26, when compared under identical conditions.

As used herein, the term “beta globin polyadenylation sequence” or “BGpA sequence” refers to an isolated transcriptional regulatory element comprising a nucleic acid sequence that is at least 75% identical to SEQ ID NO:27 and that retains the polyadenylation sequence set forth in SEQ ID NO:27.

As used herein, the term “reverse complement” has its art recognized meaning. By way of example, an hHCR1 sequence and the corresponding reverse complement hHCR1 sequence are set forth in SEQ ID NO:22 and 23, respectively.

As used herein, the term “isolated” in the context of an isolated nucleic acid sequence or polynucleotide, refers to separation from the genetic context in which the nucleic acid sequence exists in nature.

As used herein, the terms “recombinant adeno-associated virus” or “rAAV” refers to an AAV comprising a genome lacking functional rep and cap genes.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., an antibody heavy chain or light chain coding sequence operably linked to a transcriptional regulatory element), the rAAV genome can be in the sense or antisense orientation relative to the direction of transcription of the transgene.

As used herein, the term “AAV capsid protein” refers to an AAV VP1, VP2, or VP3 capsid protein.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. When a sequence is described herein as being a certain percentage identical to a reference sequence, the percentage identity to the reference sequence is determined across the full length of the reference sequence.

When a nucleic sequence is referred to herein as “consisting of” a particular sequence, the term “consisting of” is intended to limit only the sequence of the nucleotides in the nucleic sequence. It is not intended to preclude the nucleotides in the nucleic sequence from comprising modifications (e.g., methylation, acetylation, etc.) or from being linked to another molecule. For example, the term “hAAT promoter sequence consisting of SEQ ID NO:17” means that the hAAT promoter sequence contains only the nucleotide sequence set forth in SEQ ID NO:17 (and no additional sequence from an hAAT promoter). It does not preclude the nucleotides in that hAAT promoter sequence from comprising base modifications (e.g., methylation, acetylation, etc.) or from being linked to another molecule (e.g., an hHCR1 sequence).

As used herein, the term “coding sequence” refers to the portion of a complementary DNA (cDNA) that encodes a polypeptide, beginning at the start codon and ending at the stop codon.

As used herein, the term “polyadenylation sequence” refers to a DNA sequence that when transcribed into RNA constitutes a polyadenylation signal sequence, as that term is known in the art (e.g., the RNA sequence AAUAAA). A polyadenylation sequence can be, without limitation, naturally occurring or synthetic.

As used herein, the term “intron element” refers to a cis-acting nucleotide sequence comprising an intron. In certain embodiments, an intron element comprises a modified intron, e.g., a synthetic intron sequence. In certain embodiments, an intron element comprises an intron exogenous to the transgene in which it is located. In certain embodiments, an intron element comprises a modified splice acceptor and/or splice donor resulting in more robust splicing activity. While not wishing to be bound by theory, it is hypothesized that introns can increase transgene expression, for example, by reducing transcriptional silencing and enhancing mRNA export from the nucleus to the cytoplasm. A skilled worker will appreciate that synthetic intron sequences can be designed to mediate RNA splicing by introducing any consensus splicing motifs known in the art (e.g., in Sibley et al., (2016) Nature Reviews Genetics, 17, 407-21, which is incorporated by reference herein in its entirety). Exemplary intron sequences are provided in Lu et al. (2013) Molecular Therapy 21(5): 954-63, and Lu et al. (2017) Hum. Gene Ther. 28(1): 125-34, which are incorporated by reference herein in their entirety.

As used herein, the term “effective amount” in the context of the administration of a substance to a subject refers to the amount of the substance that achieves a desired therapeutic effect.

### II. Polynucleotides and Nucleic Acid Vectors

Provided herein are novel polynucleotides suitable for use in bidirectional dual expression constructs.

In one aspect, the instant disclosure provides an isolated polynucleotide comprising an hAAT promoter sequence and an hHCR1 sequence. In certain embodiments, the polynucleotide comprises from 5′ to 3′: a reverse complement hAAT promoter sequence; and an hHCR1 sequence. In certain embodiments, the polynucleotide comprises from 5′ to 3′: a reverse complement hAAT promoter sequence consisting of a sequence that is at least 85% identical to SEQ ID NO:17, and an hHCR1 sequence consisting of a sequence that is at least 85% identical to SEQ ID NO:22. In certain embodiments, the polynucleotide comprises from 5′ to 3′: a reverse complement hAAT promoter sequence consisting of a sequence that is at least 85% identical to SEQ ID NO:17, and a reverse complement hHCR1 sequence consisting of a sequence that is at least 85% identical to SEQ ID NO:23. In certain embodiments, the polynucleotide comprises from 5′ to 3′: a reverse complement hAAT promoter sequence consisting of a sequence that is at least 85% identical to SEQ ID NO:18, and an hHCR1 sequence consisting of a sequence that is at least 85% identical to SEQ ID NO:22. In certain embodiments, the polynucleotide comprises from 5′ to 3′: a reverse complement hAAT promoter sequence consisting of a sequence that is at least 85% identical to SEQ ID NO:18, and a reverse complement hHCR1 sequence consisting of a sequence that is at least 85% identical to SEQ ID NO:23. In certain embodiments, the polynucleotide comprises from 5′ to 3′: a reverse complement hAAT promoter sequence consisting of a sequence that is at least 85% identical to SEQ ID NO:20, and an hHCR1 sequence consisting of a sequence that is at least 85% identical to SEQ ID NO:22. In certain embodiments, the polynucleotide comprises from 5′ to 3′: a reverse complement hAAT promoter sequence consisting of a sequence that is at least 85% identical to SEQ ID NO:20, and a reverse complement hHCR1 sequence consisting of a sequence that is at least 85% identical to SEQ ID NO:23. In certain embodiments, the polynucleotide comprises from 5′ to 3′: a reverse complement hAAT promoter sequence consisting of a sequence that is at least 85% identical to SEQ ID NO:17, and an hHCR1 sequence consisting of a sequence that is at least 85% identical to SEQ ID NO:24. In certain embodiments, the polynucleotide comprises from 5′ to 3′: a reverse complement hAAT promoter sequence consisting of a sequence that is at least 85% identical to SEQ ID NO:18, and an hHCR1 sequence consisting of a sequence that is at least 85% identical to SEQ ID NO:24. In certain embodiments, the polynucleotide comprises from 5′ to 3′: a reverse complement hAAT promoter sequence consisting of a sequence that is at least 85% identical to SEQ ID NO:18, and a reverse complement hHCR1 sequence consisting of a sequence that is at least 85% identical to SEQ ID NO:25. In certain embodiments, the polynucleotide comprises from 5′ to 3′: a reverse complement hAAT promoter sequence consisting of a sequence that is at least 85% identical to SEQ ID NO:20, and an hHCR1 sequence consisting of a sequence that is at least 85% identical to SEQ ID NO:24. In certain embodiments, the polynucleotide comprises from 5′ to 3′: a reverse complement hAAT promoter sequence consisting of a sequence that is at least 85% identical to SEQ ID NO:20, and a reverse complement hHCR1 sequence consisting of a sequence that is at least 85% identical to SEQ ID NO:25. In certain embodiments, the polynucleotide comprises from 5′ to 3′: a reverse complement hAAT promoter sequence consisting of SEQ ID NO:17, and an hHCR1 sequence consisting of SEQ ID NO:22. In certain embodiments, the polynucleotide comprises from 5′ to 3′: a reverse complement hAAT promoter sequence consisting of SEQ ID NO:17, and a reverse complement hHCR1 sequence consisting of SEQ ID NO:23. In certain embodiments, the polynucleotide comprises from 5′ to 3′: a reverse complement hAAT promoter sequence consisting of SEQ ID NO:18, and an hHCR1 sequence consisting of SEQ ID NO:22. In certain embodiments, the polynucleotide comprises from 5′ to 3′: a reverse complement hAAT promoter sequence consisting of SEQ ID NO:18, and a reverse complement hHCR1 sequence consisting of SEQ ID NO:23. In certain embodiments, the polynucleotide comprises from 5′ to 3′: a reverse complement hAAT promoter sequence consisting of SEQ ID NO:20, and an hHCR1 sequence consisting of SEQ ID NO:22. In certain embodiments, the polynucleotide comprises from 5′ to 3′: a reverse complement hAAT promoter sequence consisting of SEQ ID NO:20, and a reverse complement hHCR1 consisting of SEQ ID NO:23. In certain embodiments, the polynucleotide comprises from 5′ to 3′: a reverse complement hAAT promoter sequence consisting of SEQ ID NO:17, and an hHCR1 sequence consisting of SEQ ID NO:24. In certain embodiments, the polynucleotide comprises from 5′ to 3′: a reverse complement hAAT promoter sequence consisting of SEQ ID NO:18, and an hHCR1 sequence consisting of SEQ ID NO:24. In certain embodiments, the polynucleotide comprises from 5′ to 3′: a reverse complement hAAT promoter sequence consisting of SEQ ID NO:18, and a reverse complement hHCR1 sequence consisting of SEQ ID NO:25. In certain embodiments, the polynucleotide comprises from 5′ to 3′: a reverse complement hAAT promoter sequence consisting of SEQ ID NO:20, and an hHCR1 sequence consisting of SEQ ID NO:24. In certain embodiments, the polynucleotide comprises from 5′ to 3′: a reverse complement hAAT promoter sequence consisting of SEQ ID NO:20, and a reverse complement hHCR1 sequence consisting of SEQ ID NO:25.

In certain embodiments, the isolated polynucleotide comprises a sequence selected from the group consisting of SEQ ID NOs:36-40 and 72.

In certain embodiments of each of the foregoing embodiments, the isolated polynucleotide further comprises a TTR promoter sequence positioned 3′ to the hHCR1 sequence or the reverse complement hHCR1 sequence. In certain embodiments, the TTR promoter sequence comprises a nucleic acid sequence that is at least 85% identical to SEQ ID NO:26. In certain embodiments, the TTR promoter sequence consists of sequence that is at least 85% identical to SEQ ID NO:26. In certain embodiments, the TTR promoter sequence comprises SEQ ID NO:26. In certain embodiments, the TTR promoter sequence consists of SEQ ID NO:26.

In another aspect, the instant disclosure provides an isolated polynucleotide comprising from 5′ to 3′: an hHCR1 sequence, and a TTR promoter sequence.

In certain embodiments, the isolated polynucleotide comprises from 5′ to 3′: an hHCR1 sequence consisting of a sequence that is at least 85% identical to SEQ ID NO:22, and a TTR promoter sequence consisting of a sequence that is at least 85% identical to SEQ ID NO:26. In certain embodiments, the isolated polynucleotide comprises from 5′ to 3′: an hHCR1 sequence consisting of SEQ ID NO:22, and a TTR promoter sequence consisting of SEQ ID NO:26.

In certain embodiments, the isolated polynucleotide comprises from 5′ to 3′: a reverse complement hHCR1 sequence consisting of a sequence that is at least 85% identical to SEQ ID NO:23, and a TTR promoter sequence consisting of a sequence that is at least 85% identical to SEQ ID NO:26. In certain embodiments, the isolated polynucleotide comprises from 5′ to 3′: a reverse complement hHCR1 sequence consisting of SEQ ID NO:23, and a TTR promoter sequence consisting of SEQ ID NO:26.

In certain embodiments, the isolated polynucleotide comprises from 5′ to 3′: an hHCR1 sequence consisting of a sequence that is at least 85% identical to SEQ ID NO:24, and a TTR promoter sequence consisting of a sequence that is at least 85% identical to SEQ ID NO:26. In certain embodiments, the isolated polynucleotide comprises from 5′ to 3′: an hHCR1 sequence consisting of SEQ ID NO:24, and a TTR promoter sequence consisting of SEQ ID NO:26.

In certain embodiments, the isolated polynucleotide comprises from 5′ to 3′: a reverse complement hHCR1 sequence consisting of a sequence that is at least 85% identical to SEQ ID NO:25, and a TTR promoter sequence consisting of a sequence that is at least 85% identical to SEQ ID NO:26. In certain embodiments, the isolated polynucleotide comprises from 5′ to 3′: a reverse complement hHCR1 sequence consisting of SEQ ID NO:25, and a TTR promoter sequence consisting of SEQ ID NO:26.

In certain embodiments, the isolated polynucleotide comprises a sequence selected from the group consisting of SEQ ID NOs:41-43.

In certain embodiments of each of the foregoing embodiments, the isolated polynucleotide further comprises a polyadenylation sequence, or a reverse complement thereof, interposed between the hHCR1 sequence or the reverse complement hHCR1 sequence and the TTR promoter sequence. In certain embodiments, the polyadenylation sequence is a BGpA sequence. In certain embodiments, the BGpA sequence comprises a sequence that is at least 85% identical to SEQ ID NO:27. In certain embodiments, the BGpA sequence consisting of a sequence that is at least 85% identical to SEQ ID NO:27. In certain embodiments, the BGpA sequence comprises SEQ ID NO:27. In certain embodiments, the BGpA sequence consists of SEQ ID NO:27.

In certain embodiments, the isolated polynucleotide comprises a sequence that is at least 85% identical to a sequence selected from the group consisting of SEQ ID NOs:60-71 and 76-78. In certain embodiments, the isolated polynucleotide comprises a sequence selected from the group consisting of SEQ ID NOs:60-71 and 76-78.

In certain embodiments of each of the foregoing embodiments, the isolated polynucleotide further comprises a first intron element positioned 5′ to the reverse complement hAAT promoter sequence. In certain embodiments of each of the foregoing embodiments, the isolated polynucleotide further comprises a second intron element positioned 3′ to the TTR promoter sequence.

Any intron element can be used as the first or second intron element in the polynucleotides disclosed herein. Intron elements can be naturally occurring or synthetic (e.g., chimeric intron elements). In certain embodiments, the first intron element comprises a sequence that is at least 85% identical to SEQ ID NO:28, 29, 30, 31, or 32, optionally wherein the first intron element comprises SEQ ID NO:28, 29, 30, 31, or 32, optionally wherein the sequence of the first intron element consists of SEQ ID NO:28, 29, 30, 31, or 32. In certain embodiments, the second intron element comprises a sequence that is at least 85% identical to SEQ ID NO:28, 29, 30, 31, or 32, optionally wherein the second intron element comprises SEQ ID NO:28, 29, 30, 31, or 32, optionally wherein the sequence of the second intron element consists of SEQ ID NO:28, 29, 30, 31, or 32.

In certain embodiments, the polynucleotides disclosed herein comprise from 5′ to 3′: (a) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:29; (b) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:30; (c) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:31; or (d) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:32.

In certain embodiments, the polynucleotides disclosed herein comprise from 5′ to 3′: (a) a first intron element comprising the sequence of SEQ ID NO:28; a nucleic sequence selected from the group consisting of SEQ ID NOs:60-71 and 76-78; and a second intron element comprising the sequence of SEQ ID NO:29; (b) a first intron element comprising the sequence of SEQ ID NO:28; a nucleic sequence selected from the group consisting of SEQ ID NOs:60-71 and 76-78; and a second intron element comprising the sequence of SEQ ID NO:30; (c) a first intron element comprising the sequence of SEQ ID NO:28; a nucleic sequence selected from the group consisting of SEQ ID NOs:60-71 and 76-78; and a second intron element comprising the sequence of SEQ ID NO:31; or (d) a first intron element comprising the sequence of SEQ ID NO:28; a nucleic sequence selected from the group consisting of SEQ ID NOs:60-71 and 76-78; and a second intron element comprising the sequence of SEQ ID NO:32.

In certain embodiments, the polynucleotides disclosed herein comprise a sequence that is at least 85% identical to a sequence selected from the group consisting of SEQ ID NOs:79-116. In certain embodiments, the polynucleotides disclosed herein comprise a sequence selected from the group consisting of SEQ ID NOs:79-116.

The foregoing polynucleotides are useful for the efficient simultaneous expression of two coding sequences. Accordingly, in certain embodiments of each of the foregoing embodiments, the polynucleotides further comprise: a reverse complement first coding sequence positioned 5′ of the reverse complement hAAT promoter sequence or the first intron element; and a second coding sequence positioned 3′ of the TTR promoter sequence of the second intron element.

In certain embodiments of each of the foregoing embodiments, the polynucleotides further comprise: a reverse complement first polyadenylation sequence positioned 5′ to the reverse complement first coding sequence; and/or a second polyadenylation sequence positioned 3′ to the second coding sequence. Any polyadenylation sequence can be employed as the first and/or second polyadenylation sequence in the polynucleotides disclosed herein. For example, in certain embodiments, the reverse complement first polyadenylation sequence comprises a sequence that is at least 85% identical to SEQ ID NO:33, 34, or 35, optionally wherein the reverse complement first polyadenylation sequence comprises SEQ ID NO:33, 34, or 35, optionally wherein the sequence of the reverse complement first polyadenylation sequence consists of SEQ ID NO:33, 34, or 35; and/or the second polyadenylation sequence comprises a sequence that is at least 85% identical to SEQ ID NO:33, 34, or 35, optionally wherein the second polyadenylation sequence comprises SEQ ID NO:33, 34, or 35, optionally wherein the sequence of the second polyadenylation sequence consists of SEQ ID NO:33, 34, or 35.

In certain embodiments, the isolated polynucleotides disclosed herein comprises from 5′ to 3′: (a) a reverse complement first polyadenylation sequence comprising a sequence that is at least 85% identical to SEQ ID NO:33; a reverse complement first coding sequence; a nucleic sequence that is at least 85% identical to a nucleic acid sequence selected from the group consisting of SEQ ID NOs:60-71 and 76-116; a second coding sequence; and a second polyadenylation sequence comprising a sequence that is at least 85% identical to SEQ ID NO:35; or (b) a reverse complement first polyadenylation sequence comprising a sequence that is at least 85% identical to SEQ ID NO:34; a reverse complement first coding sequence; a nucleic sequence that is at least 85% identical to a nucleic acid sequence selected from the group consisting of SEQ ID NOs:60-71 and 76-116; a second coding sequence; and a second polyadenylation sequence comprising a sequence that is at least 85% identical to SEQ ID NO:35. In certain embodiments, the isolated polynucleotides disclosed herein comprises from 5′ to 3′: (a) a reverse complement first polyadenylation sequence comprising the sequence of SEQ ID NO:33; a reverse complement first coding sequence; a nucleic sequence selected from the group consisting of SEQ ID NOs:60-71 and 76-116; a second coding sequence; and a second polyadenylation sequence comprising the sequence of SEQ ID NO:35; or (b) a reverse complement first polyadenylation sequence comprising the sequence of SEQ ID NO:34; a reverse complement first coding sequence; a nucleic sequence selected from the group consisting of SEQ ID NOs:60-71 and 76-116; a second coding sequence; and a second polyadenylation sequence comprising the sequence of SEQ ID NO:35.

Any two coding sequences can be expressed using the polynucleotides disclosed herein. In certain embodiments, the first and second coding sequence encode two polypeptides chain of a heteromultimeric protein, e.g., the two different polypeptide chains of an antibody or T cell receptor. In certain embodiments, the first coding sequence encodes an antibody heavy chain or an antigen-binding fragment thereof, and the second coding sequence encodes an antibody light chain or an antigen-binding fragment thereof. In certain embodiments, the first coding sequence encodes an antibody light chain or an antigen-binding fragment thereof and the second coding sequence encodes an antibody heavy chain or an antigen-binding fragment thereof.

Any antibody or an antigen-binding fragment thereof can be expressed using the polynucleotides disclosed herein. Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab. In certain embodiments, the antibody heavy chain comprises the amino acid sequence of SEQ ID NO:178, and/or the antibody light chain comprises the amino acid sequence of SEQ ID NO:179.

In certain embodiments, the first and/or second coding sequence comprise a coding sequence that has been optimized to improve expression, improve polynucleotide stability, and/or reduce immunogenicity.

In certain embodiments, the first coding sequence comprises a nucleic acid sequence selected from the group consisting of SEQ ID NOs:180-199, and the second coding sequence comprises a nucleic acid sequence selected from the group consisting of SEQ ID NOs:200-217. In certain embodiments, the first coding sequence comprises a nucleic acid sequence selected from the group consisting of SEQ ID NOs:200-217, and the second coding sequence comprises a nucleic acid sequence selected from the group consisting of SEQ ID NOs:180-199. In certain embodiments, the first coding sequence and the second coding sequence, respectively, comprise the nucleic acid sequences set forth in SEQ ID NOs:181 and 200, 191 and 209; 193 and 211, 195 and 213, 197 and 215, or 199 and 217. In certain embodiments, the second coding sequence and the first coding sequence, respectively, comprise the nucleic acid sequences set forth in SEQ ID NOs:181 and 200, 191 and 209; 193 and 211, 195 and 213, 197 and 215, or 199 and 217.

In certain embodiments, the first coding sequence comprises the sequence selected of SEQ ID NO:180, and the second coding sequence comprises a nucleic acid sequence selected from the group consisting of SEQ ID NOs:200-217. In certain embodiments, the first coding sequence comprises the sequence selected of SEQ ID NO:181, and the second coding sequence comprises a nucleic acid sequence selected from the group consisting of SEQ ID NOs:200-217. In certain embodiments, the first coding sequence comprises the sequence selected of SEQ ID NO:182, and the second coding sequence comprises a nucleic acid sequence selected from the group consisting of SEQ ID NOs:200-217. In certain embodiments, the first coding sequence comprises the sequence selected of SEQ ID NO:183, and the second coding sequence comprises a nucleic acid sequence selected from the group consisting of SEQ ID NOs:200-217. In certain embodiments, the first coding sequence comprises the sequence selected of SEQ ID NO:184, and the second coding sequence comprises a nucleic acid sequence selected from the group consisting of SEQ ID NOs:200-217. In certain embodiments, the first coding sequence comprises the sequence selected of SEQ ID NO:185, and the second coding sequence comprises a nucleic acid sequence selected from the group consisting of SEQ ID NOs:200-217. In certain embodiments, the first coding sequence comprises the sequence selected of SEQ ID NO:186, and the second coding sequence comprises a nucleic acid sequence selected from the group consisting of SEQ ID NOs:200-217. In certain embodiments, the first coding sequence comprises the sequence selected of SEQ ID NO:187, and the second coding sequence comprises a nucleic acid sequence selected from the group consisting of SEQ ID NOs:200-217. In certain embodiments, the first coding sequence comprises the sequence selected of SEQ ID NO:188, and the second coding sequence comprises a nucleic acid sequence selected from the group consisting of SEQ ID NOs:200-217. In certain embodiments, the first coding sequence comprises the sequence selected of SEQ ID NO:189, and the second coding sequence comprises a nucleic acid sequence selected from the group consisting of SEQ ID NOs:200-217. In certain embodiments, the first coding sequence comprises the sequence selected of SEQ ID NO:190, and the second coding sequence comprises a nucleic acid sequence selected from the group consisting of SEQ ID NOs:200-217. In certain embodiments, the first coding sequence comprises the sequence selected of SEQ ID NO:191, and the second coding sequence comprises a nucleic acid sequence selected from the group consisting of SEQ ID NOs:200-217. In certain embodiments, the first coding sequence comprises the sequence selected of SEQ ID NO:192, and the second coding sequence comprises a nucleic acid sequence selected from the group consisting of SEQ ID NOs:200-217. In certain embodiments, the first coding sequence comprises the sequence selected of SEQ ID NO:193, and the second coding sequence comprises a nucleic acid sequence selected from the group consisting of SEQ ID NOs:200-217. In certain embodiments, the first coding sequence comprises the sequence selected of SEQ ID NO:194, and the second coding sequence comprises a nucleic acid sequence selected from the group consisting of SEQ ID NOs:200-217. In certain embodiments, the first coding sequence comprises the sequence selected of SEQ ID NO:195, and the second coding sequence comprises a nucleic acid sequence selected from the group consisting of SEQ ID NOs:200-217. In certain embodiments, the first coding sequence comprises the sequence selected of SEQ ID NO:196, and the second coding sequence comprises a nucleic acid sequence selected from the group consisting of SEQ ID NOs:200-217. In certain embodiments, the first coding sequence comprises the sequence selected of SEQ ID NO:197, and the second coding sequence comprises a nucleic acid sequence selected from the group consisting of SEQ ID NOs:200-217. In certain embodiments, the first coding sequence comprises the sequence selected of SEQ ID NO:198, and the second coding sequence comprises a nucleic acid sequence selected from the group consisting of SEQ ID NOs:200-217. In certain embodiments, the first coding sequence comprises the sequence selected of SEQ ID NO:199, and the second coding sequence comprises a nucleic acid sequence selected from the group consisting of SEQ ID NOs:200-217. In certain embodiments, the second coding sequence comprises the sequence selected of SEQ ID NO:180, and the first coding sequence comprises a nucleic acid sequence selected from the group consisting of SEQ ID NOs:200-217. In certain embodiments, the second coding sequence comprises the sequence selected of SEQ ID NO:181, and the first coding sequence comprises a nucleic acid sequence selected from the group consisting of SEQ ID NOs:200-217. In certain embodiments, the second coding sequence comprises the sequence selected of SEQ ID NO:182, and the first coding sequence comprises a nucleic acid sequence selected from the group consisting of SEQ ID NOs:200-217. In certain embodiments, the second coding sequence comprises the sequence selected of SEQ ID NO:183, and the first coding sequence comprises a nucleic acid sequence selected from the group consisting of SEQ ID NOs:200-217. In certain embodiments, the second coding sequence comprises the sequence selected of SEQ ID NO:184, and the first coding sequence comprises a nucleic acid sequence selected from the group consisting of SEQ ID NO:200-217. In certain embodiments, the second coding sequence comprises the sequence selected of SEQ ID NO:185, and the first coding sequence comprises a nucleic acid sequence selected from the group consisting of SEQ ID NOs:200-217. In certain embodiments, the second coding sequence comprises the sequence selected of SEQ ID NO:186, and the first coding sequence comprises a nucleic acid sequence selected from the group consisting of SEQ ID NOs:200-217. In certain embodiments, the second coding sequence comprises the sequence selected of SEQ ID NO:187, and the first coding sequence comprises a nucleic acid sequence selected from the group consisting of SEQ ID NOs:200-217. In certain embodiments, the second coding sequence comprises the sequence selected of SEQ ID NO:188, and the first coding sequence comprises a nucleic acid sequence selected from the group consisting of SEQ ID NOs:200-217. In certain embodiments, the second coding sequence comprises the sequence selected of SEQ ID NO:189, and the first coding sequence comprises a nucleic acid sequence selected from the group consisting of SEQ ID NOs:200-217. In certain embodiments, the second coding sequence comprises the sequence selected of SEQ ID NO:190, and the first coding sequence comprises a nucleic acid sequence selected from the group consisting of SEQ ID NOs:200-217. In certain embodiments, the second coding sequence comprises the sequence selected of SEQ ID NO:191, and the first coding sequence comprises a nucleic acid sequence selected from the group consisting of SEQ ID NOs:200-217. In certain embodiments, the second coding sequence comprises the sequence selected of SEQ ID NO:192, and the first coding sequence comprises a nucleic acid sequence selected from the group consisting of SEQ ID NOs:200-217. In certain embodiments, the second coding sequence comprises the sequence selected of SEQ ID NO:193, and the first coding sequence comprises a nucleic acid sequence selected from the group consisting of SEQ ID NOs:200-217. In certain embodiments, the second coding sequence comprises the sequence selected of SEQ ID NO:194, and the first coding sequence comprises a nucleic acid sequence selected from the group consisting of SEQ ID NOs:200-217. In certain embodiments, the second coding sequence comprises the sequence selected of SEQ ID NO:195, and the first coding sequence comprises a nucleic acid sequence selected from the group consisting of SEQ ID NOs:200-217. In certain embodiments, the second coding sequence comprises the sequence selected of SEQ ID NO:196, and the first coding sequence comprises a nucleic acid sequence selected from the group consisting of SEQ ID NOs:200-217. In certain embodiments, the second coding sequence comprises the sequence selected of SEQ ID NO:197, and the first coding sequence comprises a nucleic acid sequence selected from the group consisting of SEQ ID NOs:200-217. In certain embodiments, the second coding sequence comprises the sequence selected of SEQ ID NO:198, and the first coding sequence comprises a nucleic acid sequence selected from the group consisting of SEQ ID NOs:200-217. In certain embodiments, the second coding sequence comprises the sequence selected of SEQ ID NO:199, and the first coding sequence comprises a nucleic acid sequence selected from the group consisting of SEQ ID NOs:200-217.

In certain embodiments, the isolated polynucleotide comprises from 5′ to 3′: SEQ ID NO:33; the reverse complement of SEQ ID NO:191; the reverse complement of SEQ ID NO:172, a nucleic sequence selected from the group consisting of SEQ ID NOs:60-71 and 76-116; SEQ ID NO:177; SEQ ID NO:209; and SEQ ID NO:35. In certain embodiments, the isolated polynucleotide comprises from 5′ to 3′: SEQ ID NO:34; the reverse complement of SEQ ID NO:181; the reverse complement of SEQ ID NO:168, a nucleic sequence selected from the group consisting of SEQ ID NOs:60-71 and 76-116; SEQ ID NO:174; SEQ ID NO:200; and SEQ ID NO:35. In certain embodiments, the isolated polynucleotide comprises from 5′ to 3′: SEQ ID NO:34; the reverse complement of SEQ ID NO:191; the reverse complement of SEQ ID NO:172; a nucleic sequence selected from the group consisting of SEQ ID NOs:60-71 and 76-116; SEQ ID NO:177; SEQ ID NO:209; and SEQ ID NO:35. In certain embodiments, the isolated polynucleotide comprises from 5′ to 3′: SEQ ID NO:34; the reverse complement of SEQ ID NO:193; the reverse complement of SEQ ID NO:172, a nucleic sequence selected from the group consisting of SEQ ID NOs:60-71 and 76-116; SEQ ID NO:177; SEQ ID NO:211; and SEQ ID NO:35. In certain embodiments, the isolated polynucleotide comprises from 5′ to 3′: SEQ ID NO:34; the reverse complement of SEQ ID NO:195; the reverse complement of SEQ ID NO:172, a nucleic sequence selected from the group consisting of SEQ ID NOs:60-71 and 76-116; SEQ ID NO:177; SEQ ID NO:213; and SEQ ID NO:35. In certain embodiments, the isolated polynucleotide comprises from 5′ to 3′: SEQ ID NO:34; the reverse complement of SEQ ID NO:197; the reverse complement of SEQ ID NO:172, a nucleic sequence selected from the group consisting of SEQ ID NOs:60-71 and 76-116; SEQ ID NO:177; SEQ ID NO:215; and SEQ ID NO:35. In certain embodiments, the isolated polynucleotide comprises from 5′ to 3′: SEQ ID NO:34; the reverse complement of SEQ ID NO:199; the reverse complement of SEQ ID NO:172, a nucleic sequence selected from the group consisting of SEQ ID NOs:60-71 and 76-116; SEQ ID NO:177; SEQ ID NO:217; and SEQ ID NO:35.

In certain embodiments, the isolated polynucleotide comprises from 5′ to 3′: SEQ ID NO:33; the reverse complement of SEQ ID NO:209; the reverse complement of SEQ ID NO:177, a nucleic sequence selected from the group consisting of SEQ ID NOs:60-71 and 76-116; SEQ ID NO:172; SEQ ID NO:191; and SEQ ID NO:35. In certain embodiments, the isolated polynucleotide comprises from 5′ to 3′: SEQ ID NO:34; the reverse complement of SEQ ID NO:200; the reverse complement of SEQ ID NO:174, a nucleic sequence selected from the group consisting of SEQ ID NOs:60-71 and 76-116; SEQ ID NO:168; SEQ ID NO:181; and SEQ ID NO:35. In certain embodiments, the isolated polynucleotide comprises from 5′ to 3′: SEQ ID NO:34; the reverse complement of SEQ ID NO:209; the reverse complement of SEQ ID NO:177, a nucleic sequence selected from the group consisting of SEQ ID NOs:60-71 and 76-116; SEQ ID NO:172; SEQ ID NO:191; and SEQ ID NO:35. In certain embodiments, the isolated polynucleotide comprises from 5′ to 3′: SEQ ID NO:34; the reverse complement of SEQ ID NO:211; the reverse complement of SEQ ID NO:177, a nucleic sequence selected from the group consisting of SEQ ID NOs:60-71 and 76-116; SEQ ID NO:172; SEQ ID NO:193; and SEQ ID NO:35. In certain embodiments, the isolated polynucleotide comprises from 5′ to 3′: SEQ ID NO:34; the reverse complement of SEQ ID NO:213; the reverse complement of SEQ ID NO:177, a nucleic sequence selected from the group consisting of SEQ ID NOs:60-71 and 76-116; SEQ ID NO:172; SEQ ID NO:195; and SEQ ID NO:35. In certain embodiments, the isolated polynucleotide comprises from 5′ to 3′: SEQ ID NO:34; the reverse complement of SEQ ID NO:215; the reverse complement of SEQ ID NO:177, a nucleic sequence selected from the group consisting of SEQ ID NOs:60-71 and 76-116; SEQ ID NO:172; SEQ ID NO:197; and SEQ ID NO:35. In certain embodiments, the isolated polynucleotide comprises from 5′ to 3′: SEQ ID NO:34; the reverse complement of SEQ ID NO:217; the reverse complement of SEQ ID NO:177, a nucleic sequence selected from the group consisting of SEQ ID NOs:60-71 and 76-116; SEQ ID NO:172; SEQ ID NO:199; and SEQ ID NO:35.

In certain embodiments, the first coding sequence and/or the second coding sequence comprises a signal sequence. Any signal sequence can direct secretion of a polypeptide from a mammalian cell is suitable. In certain embodiments, the signal sequence comprises the amino acid sequence of SEQ ID NO:167 or 173. In certain embodiments, the first coding sequence and/or the second coding sequence comprise a nucleic acid sequence selected from the group consisting of SEQ ID NOs:168-172 and 174-177.

In certain embodiments, the instant disclosure provides polynucleotides comprising from 5′ to 3′: SEQ ID NO:168 and any one of SEQ ID NOs:180-217; SEQ ID NO:169 and any one of SEQ ID NOs:180-217; SEQ ID NO:170 and any one of SEQ ID NOs:180-217; SEQ ID NO:171 and any one of SEQ ID NOs:180-217; SEQ ID NO:172 and any one of SEQ ID NOs:180-217; SEQ ID NO:174 and any one of SEQ ID NOs:180-217; SEQ ID NO:175 and any one of SEQ ID NOs:180-217; SEQ ID NO:176 and any one of SEQ ID NOs:180-217; or SEQ ID NO:177 and any one of SEQ ID NOs:180-217.

In certain embodiments, the instant disclosure provides a polynucleotide comprising a nucleic acid sequence selected from the group consisting of SEQ ID NOs:19, 29-31, 44-59, 117-140, 168-172, 174-177 and 180-217.

In certain embodiments, the instant disclosure provides polynucleotides comprising an hAAT promoter sequence consisting of a sequence that is at least 85% identical to SEQ ID NO:21, or 22, optionally wherein the hAAT promoter sequence consists of SEQ ID NO:21, or 22.

In certain embodiments, the instant disclosure provides polynucleotides comprising an hAAT promoter sequence comprising SEQ ID NO:19, optionally wherein the hAAT promoter sequence consists of SEQ ID NO:19.

In certain embodiments, the polynucleotide is comprised within an rAAV genome, the rAAV genome comprising a 5′ inverted terminal repeat (5′ ITR) sequence, and a 3′ inverted terminal repeat (3′ ITR) sequence. Any 5′ and 3′ AAV ITR sequences can be employed in the rAAV genome. In certain embodiments, the 5′ ITR nucleic acid sequence is at least 85% identical to the nucleic acid sequence set forth in SEQ ID NO:165, and/or the 3′ ITR nucleic acid sequence is at least 85% identical to the nucleic acid sequence set forth in SEQ ID NO:166. In certain embodiments, the 5′ ITR nucleic acid sequence comprises the nucleic acid sequence set forth in SEQ ID NO:165, and/or the 3′ ITR nucleic acid sequence comprises the nucleic acid sequence set forth in SEQ ID NO:166. In certain embodiments, the rAAV genome comprises a nucleic acid sequence that is at least 85% to the nucleic acid sequence set forth in SEQ ID NOs:141-164, or the complement thereof. Exemplary rAAV genomes are set forth in Table 1 herein. Individual sequence elements (e.g., hAAT promoter sequence, hHCR sequence, etc.) present in any of the rAAV genomes in Table 1 can be contiguous or can have additional nucleotides interposed between the sequence elements. In certain embodiments, at least 2 (e.g., 2, 3, 4, 5, 6, 7, 8, 9, 10, 11, or 12) of the individual sequence elements are contiguous. In certain embodiments, all of the individual sequence elements are contiguous.

In certain embodiments, the rAAV genome comprises from 5′ to 3′: SEQ ID NO:34; the reverse complement of SEQ ID NO:181; the reverse complement of SEQ ID NO:168; SEQ ID NO:28; SEQ ID NO:17; SEQ ID NO:25; SEQ ID NO:26; SEQ ID NO:32; SEQ ID NO:174; SEQ ID NO:200; and SEQ ID NO:35. In certain embodiments, the rAAV genome comprises from 5′ to 3′: SEQ ID NO:34; the reverse complement of SEQ ID NO:181; the reverse complement of SEQ ID NO:168; SEQ ID NO:28; SEQ ID NO:17; SEQ ID NO:22; SEQ ID NO:26; SEQ ID NO:32; SEQ ID NO:174; SEQ ID NO:200; and SEQ ID NO:35. In certain embodiments, the rAAV genome comprises from 5′ to 3′: SEQ ID NO:34; the reverse complement of SEQ ID NO:181; the reverse complement of SEQ ID NO:168; SEQ ID NO:28; SEQ ID NO:17; SEQ ID NO:23; SEQ ID NO:26; SEQ ID NO:32; SEQ ID NO:174; SEQ ID NO:200; and SEQ ID NO:35. In certain embodiments, the rAAV genome comprises from 5′ to 3′: SEQ ID NO:34; the reverse complement of SEQ ID NO:181; the reverse complement of SEQ ID NO:168; SEQ ID NO:28; SEQ ID NO:20; SEQ ID NO:22; SEQ ID NO:26; SEQ ID NO:32; SEQ ID NO:174; SEQ ID NO:200; and SEQ ID NO:35. In certain embodiments, the rAAV genome comprises from 5′ to 3′: SEQ ID NO:34; the reverse complement of SEQ ID NO:181; the reverse complement of SEQ ID NO:168; SEQ ID NO:28; SEQ ID NO:20; SEQ ID NO:23; SEQ ID NO:26; SEQ ID NO:32; SEQ ID NO:174; SEQ ID NO:200; and SEQ ID NO:35. In certain embodiments, the rAAV genome comprises from 5′ to 3′: SEQ ID NO:34; the reverse complement of SEQ ID NO:181; the reverse complement of SEQ ID NO:168; SEQ ID NO:28; SEQ ID NO:17; SEQ ID NO:25; SEQ ID NO:27; SEQ ID NO:26; SEQ ID NO:32; SEQ ID NO:174; SEQ ID NO:200; and SEQ ID NO:35. In certain embodiments, the rAAV genome comprises from 5′ to 3′: SEQ ID NO:34; the reverse complement of SEQ ID NO:191; the reverse complement of SEQ ID NO:172; SEQ ID NO:28; SEQ ID NO:17; SEQ ID NO:22; SEQ ID NO:26; SEQ ID NO:32; SEQ ID NO:177; SEQ ID NO:209; and SEQ ID NO:35. In certain embodiments, the rAAV genome comprises from 5′ to 3′: SEQ ID NO:34; the reverse complement of SEQ ID NO:193; the reverse complement of SEQ ID NO:172; SEQ ID NO:28; SEQ ID NO:17; SEQ ID NO:22; SEQ ID NO:26; SEQ ID NO:32; SEQ ID NO:177; SEQ ID NO:211; and SEQ ID NO:35. In certain embodiments, the rAAV genome comprises from 5′ to 3′: SEQ ID NO:34; the reverse complement of SEQ ID NO:195; the reverse complement of SEQ ID NO:172; SEQ ID NO:28; SEQ ID NO:17; SEQ ID NO:22; SEQ ID NO:26; SEQ ID NO:32; SEQ ID NO:177; SEQ ID NO:213; and SEQ ID NO:35. In certain embodiments, the rAAV genome comprises from 5′ to 3′: SEQ ID NO:34; the reverse complement of SEQ ID NO:197; the reverse complement of SEQ ID NO:172; SEQ ID NO:28; SEQ ID NO:17; SEQ ID NO:22; SEQ ID NO:26; SEQ ID NO:32; SEQ ID NO:177; SEQ ID NO:215; and SEQ ID NO:35. In certain embodiments, the rAAV genome comprises from 5′ to 3′: SEQ ID NO:34; the reverse complement of SEQ ID NO:199; the reverse complement of SEQ ID NO:172; SEQ ID NO:28; SEQ ID NO:17; SEQ ID NO:22; SEQ ID NO:26; SEQ ID NO:32; SEQ ID NO:177; SEQ ID NO:217; and SEQ ID NO:35. In certain embodiments, the rAAV genome comprises from 5′ to 3′: SEQ ID NO:34; the reverse complement of SEQ ID NO:191; the reverse complement of SEQ ID NO:172; SEQ ID NO:28; SEQ ID NO:17; SEQ ID NO:25; SEQ ID NO:26; SEQ ID NO:32; SEQ ID NO:177; SEQ ID NO:209; and SEQ ID NO:35. In certain embodiments, the rAAV genome comprises from 5′ to 3′: SEQ ID NO:34; the reverse complement of SEQ ID NO:191; the reverse complement of SEQ ID NO:172; SEQ ID NO:28; SEQ ID NO:17; SEQ ID NO:23; SEQ ID NO:26; SEQ ID NO:32; SEQ ID NO:177; SEQ ID NO:209; and SEQ ID NO:35. In certain embodiments, the rAAV genome comprises from 5′ to 3′: SEQ ID NO:34; the reverse complement of SEQ ID NO:191; the reverse complement of SEQ ID NO:172; SEQ ID NO:28; SEQ ID NO:20; SEQ ID NO:22; SEQ ID NO:26; SEQ ID NO:32; SEQ ID NO:177; SEQ ID NO:209; and SEQ ID NO:35. In certain embodiments, the rAAV genome comprises from 5′ to 3′: SEQ ID NO:34; the reverse complement of SEQ ID NO:191; the reverse complement of SEQ ID NO:172; SEQ ID NO:28; SEQ ID NO:20; SEQ ID NO:23; SEQ ID NO:26; SEQ ID NO:32; SEQ ID NO:177; SEQ ID NO:209; and SEQ ID NO:35. In certain embodiments, the rAAV genome comprises from 5′ to 3′: SEQ ID NO:33; the reverse complement of SEQ ID NO:191; the reverse complement of SEQ ID NO:172; SEQ ID NO:28; SEQ ID NO:18; SEQ ID NO:22; SEQ ID NO:26; SEQ ID NO:31; SEQ ID NO:177; SEQ ID NO:209; and SEQ ID NO:35. In certain embodiments, the rAAV genome comprises from 5′ to 3′: SEQ ID NO:33; the reverse complement of SEQ ID NO:191; the reverse complement of SEQ ID NO:172; SEQ ID NO:28; SEQ ID NO:17; SEQ ID NO:22; SEQ ID NO:26; SEQ ID NO:31; SEQ ID NO:177; SEQ ID NO:209; and SEQ ID NO:35. In certain embodiments, the rAAV genome comprises from 5′ to 3′: SEQ ID NO:33; the reverse complement of SEQ ID NO:191; the reverse complement of SEQ ID NO:172; SEQ ID NO:28; SEQ ID NO:17; SEQ ID NO:22; SEQ ID NO:26; SEQ ID NO:32; SEQ ID NO:177; SEQ ID NO:209; and SEQ ID NO:35. In certain embodiments, the rAAV genome comprises from 5′ to 3′: SEQ ID NO:34; the reverse complement of SEQ ID NO:191; the reverse complement of SEQ ID NO:172; SEQ ID NO:28; SEQ ID NO:17; SEQ ID NO:22; SEQ ID NO:26; SEQ ID NO:29; SEQ ID NO:177; SEQ ID NO:209; and SEQ ID NO:35. In certain embodiments, the rAAV genome comprises from 5′ to 3′: SEQ ID NO:33; the reverse complement of SEQ ID NO:191; the reverse complement of SEQ ID NO:172; SEQ ID NO:28; SEQ ID NO:17; SEQ ID NO:22; SEQ ID NO:26; SEQ ID NO:29; SEQ ID NO:177; SEQ ID NO:209; and SEQ ID NO:35. In certain embodiments, the rAAV genome comprises from 5′ to 3′: SEQ ID NO:34; the reverse complement of SEQ ID NO:181; the reverse complement of SEQ ID NO:172; SEQ ID NO:28; SEQ ID NO:17; SEQ ID NO:25; SEQ ID NO:26; SEQ ID NO:32; SEQ ID NO:177; SEQ ID NO:200; and SEQ ID NO:35. In certain embodiments, the rAAV genome comprises from 5′ to 3′: SEQ ID NO:33; the reverse complement of SEQ ID NO:191; the reverse complement of SEQ ID NO:172; SEQ ID NO:28; SEQ ID NO:17; SEQ ID NO:22; SEQ ID NO:27; SEQ ID NO:26; SEQ ID NO:29; SEQ ID NO:177; SEQ ID NO:209; and SEQ ID NO:35. In certain embodiments, the rAAV genome comprises from 5′ to 3′: SEQ ID NO:34; the reverse complement of SEQ ID NO:191; the reverse complement of SEQ ID NO:172; SEQ ID NO:28; SEQ ID NO:17; SEQ ID NO:25; SEQ ID NO:27; SEQ ID NO:26; SEQ ID NO:29; SEQ ID NO:177; SEQ ID NO:209; and SEQ ID NO:35. In certain embodiments, the rAAV genome comprises from 5′ to 3′: SEQ ID NO:34; the reverse complement of SEQ ID NO:191; the reverse complement of SEQ ID NO:172; SEQ ID NO:28; SEQ ID NO:20; SEQ ID NO:25; SEQ ID NO:27; SEQ ID NO:26; SEQ ID NO:29; SEQ ID NO:177; SEQ ID NO:209; and SEQ ID NO:35.

In certain embodiments, the rAAV genome comprises the nucleic acid sequence set forth in SEQ ID NOs:141-164, or the complement thereof. In certain embodiments, the rAAV genome is a single stranded rAAV genome. In certain embodiments, the rAAV genome is a self-complementary rAAV genome.

TABLE 1
Exemplary rAAV genomes

| | | | | | | | | | | | | | Genome | Full |
|---|---|---|---|---|---|---|---|---|---|---|---|---|---|---|
| | SEQ ID NOs of Major Sequence Elements (right to left = 5′ to 3′) | | | | | | | | | | | | sequence | genome |
| Genome | (RC denotes the reverse complement of the sequence in the given SEQ ID NO) | | | | | | | | | | | | minus ITRs | sequence |
| E1-100 | 34 | RC of 181 | RC of 168 | 28 | 17 | 25 | 27 | 218 | 32 | 174 | 200 | 35 | — | 219 |
| E24 | 34 | RC of 181 | RC of 168 | 28 | 17 | 25 | None | 26 | 32 | 174 | 200 | 35 | 117 | 141 |
| E27 | 34 | RC of 181 | RC of 168 | 28 | 17 | 22 | None | 26 | 32 | 174 | 200 | 35 | 118 | 142 |
| E28 | 34 | RC of 181 | RC of 168 | 28 | 17 | 23 | None | 26 | 32 | 174 | 200 | 35 | 119 | 143 |
| E31 | 34 | RC of 181 | RC of 168 | 28 | 20 | 22 | None | 26 | 32 | 174 | 200 | 35 | 120 | 144 |
| E32 | 34 | RC of 181 | RC of 168 | 28 | 20 | 23 | None | 26 | 32 | 174 | 200 | 35 | 121 | 145 |
| E37 | 34 | RC of 181 | RC of 168 | 28 | 17 | 25 | 27 | 26 | 32 | 174 | 200 | 35 | 122 | 146 |
| E40 | 34 | RC of 191 | RC of 172 | 28 | 17 | 22 | None | 26 | 32 | 177 | 209 | 35 | 123 | 147 |
| E41 | 34 | RC of 193 | RC of 172 | 28 | 17 | 22 | None | 26 | 32 | 177 | 211 | 35 | 124 | 148 |
| E42 | 34 | RC of 195 | RC of 172 | 28 | 17 | 22 | None | 26 | 32 | 177 | 213 | 35 | 125 | 149 |
| E43 | 34 | RC of 197 | RC of 172 | 28 | 17 | 22 | None | 26 | 32 | 177 | 215 | 35 | 126 | 150 |
| E44 | 34 | RC of 199 | RC of 172 | 28 | 17 | 22 | None | 26 | 32 | 177 | 217 | 35 | 127 | 151 |
| E45 | 34 | RC of 191 | RC of 172 | 28 | 17 | 25 | None | 26 | 32 | 177 | 209 | 35 | 128 | 152 |
| E46 | 34 | RC of 191 | RC of 172 | 28 | 17 | 23 | None | 26 | 32 | 177 | 209 | 35 | 129 | 153 |
| E47 | 34 | RC of 191 | RC of 172 | 28 | 20 | 22 | None | 26 | 32 | 177 | 209 | 35 | 130 | 154 |
| E48 | 34 | RC of 191 | RC of 172 | 28 | 20 | 23 | None | 26 | 32 | 177 | 209 | 35 | 131 | 155 |
| E49 | 33 | RC of 191 | RC of 172 | 28 | 18 | 22 | None | 26 | 31 | 177 | 209 | 35 | 132 | 156 |
| E50 | 33 | RC of 191 | RC of 172 | 28 | 17 | 22 | None | 26 | 31 | 177 | 209 | 35 | 133 | 157 |
| E51 | 33 | RC of 191 | RC of 172 | 28 | 17 | 22 | None | 26 | 32 | 177 | 209 | 35 | 134 | 158 |
| E52 | 34 | RC of 191 | RC of 172 | 28 | 17 | 22 | None | 26 | 29 | 177 | 209 | 35 | 135 | 159 |
| E53 | 33 | RC of 191 | RC of 172 | 28 | 17 | 22 | None | 26 | 29 | 177 | 209 | 35 | 136 | 160 |
| E54 | 34 | RC of 181 | RC of 168 | 28 | 17 | 25 | None | 26 | 32 | 177 | 200 | 35 | 137 | 161 |
| E55 | 33 | RC of 191 | RC of 172 | 28 | 17 | 22 | 27 | 26 | 29 | 177 | 209 | 35 | 138 | 162 |
| E56 | 34 | RC of 191 | RC of 172 | 28 | 17 | 25 | 27 | 26 | 29 | 177 | 209 | 35 | 139 | 163 |
| E57 | 34 | RC of 191 | RC of 172 | 28 | 20 | 22 | 27 | 26 | 29 | 177 | 209 | 35 | 140 | 164 |
| E97 | 220 | RC of 191 | RC of 172 | 28 | 17 | 22 | None | 26 | 29 | 177 | 209 | 35 | 221 | 222 |

The polynucleotides or rAAV genomes disclosed herein can be double stranded or single stranded. In certain embodiments, the instant disclosure provides a polynucleotide that is the complement of the polynucleotide of any one of the preceding embodiments.

In certain embodiments, the instant disclosure provides nucleic acid vectors comprising the isolated polynucleotide or rAAV genome of any one of the preceding embodiments. Any nucleic vector that can accommodate the polynucleotide or rAAV genome of any one of the preceding embodiments is suitable. In certain embodiments, the nucleic acid vector is a plasmid, a virus, or a DNA minimal vector. Suitable DNA minimal vectors include, without limitation, linear covalently closed DNA (e.g., ministring DNA), linear covalently closed dumbbell shaped DNA (e.g., doggybone DNA, dumbbell DNA), minicircles, Nanoplasmids™, minimalistic immunologically defined gene expression (MIDGE) vectors, and others known to those of skill in the art. DNA minimal vectors and their methods of production are described in, e.g., U.S. Patent Application Nos. 20100233814, 20120282283, 20130216562, 20150218565, 20150218586, 20160008488, 20160215296, 20160355827, 20190185924, 20200277624, and 20210010021, all of which are herein incorporated by reference in their entireties.

### III. Adeno-Associated Virus Compositions

In another aspect, the instant disclosure provides an rAAV comprising: an AAV capsid comprising an AAV capsid protein; and an rAAV genome disclosed herein.

Any capsid protein can be used in the rAAV disclosed herein, including, without limitation, AAV1, AAV2, AAV3, AAV4, AAV5, AAV6, AAV7, AAV8, AAV9, AAVrh10, AAVrh74, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PHP.S.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of any one of SEQ ID NOs:1-15. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of any one of SEQ ID NOs:1-15, wherein: the amino acid in the capsid protein corresponding to amino acid 206 of SEQ ID NO:15 is C; the amino acid in the capsid protein corresponding to amino acid 296 of SEQ ID NO:15 is H; the amino acid in the capsid protein corresponding to amino acid 312 of SEQ ID NO:15 is Q; the amino acid in the capsid protein corresponding to amino acid 346 of SEQ ID NO:15 is A; the amino acid in the capsid protein corresponding to amino acid 464 of SEQ ID NO:15 is N; the amino acid in the capsid protein corresponding to amino acid 468 of SEQ ID NO:15 is S; the amino acid in the capsid protein corresponding to amino acid 501 of SEQ ID NO:15 is I; the amino acid in the capsid protein corresponding to amino acid 505 of SEQ ID NO:15 is R; the amino acid in the capsid protein corresponding to amino acid 590 of SEQ ID NO:15 is R; the amino acid in the capsid protein corresponding to amino acid 626 of SEQ ID NO:15 is G or Y; the amino acid in the capsid protein corresponding to amino acid 681 of SEQ ID NO:15 is M; the amino acid in the capsid protein corresponding to amino acid 687 of SEQ ID NO:15 is R; the amino acid in the capsid protein corresponding to amino acid 690 of SEQ ID NO:15 is K; the amino acid in the capsid protein corresponding to amino acid 706 of SEQ ID NO:15 is C; or, the amino acid in the capsid protein corresponding to amino acid 718 of SEQ ID NO:15 is G.

In certain embodiments, the amino acid in the capsid protein corresponding to amino acid 626 of SEQ ID NO:16 is G, and the amino acid in the capsid protein corresponding to amino acid 718 of SEQ ID NO:16 is G. In certain embodiments, the amino acid in the capsid protein corresponding to amino acid 296 of SEQ ID NO:16 is H, the amino acid in the capsid protein corresponding to amino acid 464 of SEQ ID NO:16 is N, the amino acid in the capsid protein corresponding to amino acid 505 of SEQ ID NO:16 is R, and the amino acid in the capsid protein corresponding to amino acid 681 of SEQ ID NO:16 is M. In certain embodiments, the amino acid in the capsid protein corresponding to amino acid 505 of SEQ ID NO:16 is R, and the amino acid in the capsid protein corresponding to amino acid 687 of SEQ ID NO:16 is R. In certain embodiments, the amino acid in the capsid protein corresponding to amino acid 346 of SEQ ID NO:16 is A, and the amino acid in the capsid protein corresponding to amino acid 505 of SEQ ID NO:16 is R. In certain embodiments, the amino acid in the capsid protein corresponding to amino acid 501 of SEQ ID NO:16 is I, the amino acid in the capsid protein corresponding to amino acid 505 of SEQ ID NO:16 is R, and the amino acid in the capsid protein corresponding to amino acid 706 of SEQ ID NO:16 is C. In certain embodiments, the capsid protein comprises the amino acid sequence of amino acids 203-736 of any one of SEQ ID NOs:1-15.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of any one of SEQ ID NOs:1-15. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of any one of SEQ ID NOs:1-15, wherein: the amino acid in the capsid protein corresponding to amino acid 151 of SEQ ID NO:16 is R; the amino acid in the capsid protein corresponding to amino acid 160 of SEQ ID NO:16 is D; the amino acid in the capsid protein corresponding to amino acid 206 of SEQ ID NO:16 is C; the amino acid in the capsid protein corresponding to amino acid 296 of SEQ ID NO:16 is H; the amino acid in the capsid protein corresponding to amino acid 312 of SEQ ID NO:16 is Q; the amino acid in the capsid protein corresponding to amino acid 346 of SEQ ID NO:16 is A; the amino acid in the capsid protein corresponding to amino acid 464 of SEQ ID NO:16 is N; the amino acid in the capsid protein corresponding to amino acid 468 of SEQ ID NO:16 is S; the amino acid in the capsid protein corresponding to amino acid 501 of SEQ ID NO:16 is I; the amino acid in the capsid protein corresponding to amino acid 505 of SEQ ID NO:16 is R; the amino acid in the capsid protein corresponding to amino acid 590 of SEQ ID NO:16 is R; the amino acid in the capsid protein corresponding to amino acid 626 of SEQ ID NO:16 is G or Y; the amino acid in the capsid protein corresponding to amino acid 681 of SEQ ID NO:16 is M; the amino acid in the capsid protein corresponding to amino acid 687 of SEQ ID NO:16 is R; the amino acid in the capsid protein corresponding to amino acid 690 of SEQ ID NO:16 is K; the amino acid in the capsid protein corresponding to amino acid 706 of SEQ ID NO:16 is C; or, the amino acid in the capsid protein corresponding to amino acid 718 of SEQ ID NO:16 is G. In certain embodiments, the amino acid in the capsid protein corresponding to amino acid 626 of SEQ ID NO:16 is G, and the amino acid in the capsid protein corresponding to amino acid 718 of SEQ ID NO:16 is G. In certain embodiments, the amino acid in the capsid protein corresponding to amino acid 296 of SEQ ID NO:16 is H, the amino acid in the capsid protein corresponding to amino acid 464 of SEQ ID NO:16 is N, the amino acid in the capsid protein corresponding to amino acid 505 of SEQ ID NO:16 is R, and the amino acid in the capsid protein corresponding to amino acid 681 of SEQ ID NO:16 is M. In certain embodiments, the amino acid in the capsid protein corresponding to amino acid 505 of SEQ ID NO:16 is R, and the amino acid in the capsid protein corresponding to amino acid 687 of SEQ ID NO:16 is R. In certain embodiments, the amino acid in the capsid protein corresponding to amino acid 346 of SEQ ID NO:16 is A, and the amino acid in the capsid protein corresponding to amino acid 505 of SEQ ID NO:16 is R. In certain embodiments, the amino acid in the capsid protein corresponding to amino acid 501 of SEQ ID NO:16 is I, the amino acid in the capsid protein corresponding to amino acid 505 of SEQ ID NO:16 is R, and the amino acid in the capsid protein corresponding to amino acid 706 of SEQ ID NO:16 is C. In certain embodiments, the capsid protein comprises the amino acid sequence of amino acids 138-736 of any one of SEQ ID NOs:1-15.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of any one of SEQ ID NOs:1-15. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of any one of SEQ ID NOs:1-15, wherein: the amino acid in the capsid protein corresponding to amino acid 2 of SEQ ID NO:16 is T; the amino acid in the capsid protein corresponding to amino acid 65 of SEQ ID NO:16 is I; the amino acid in the capsid protein corresponding to amino acid 68 of SEQ ID NO:16 is V; the amino acid in the capsid protein corresponding to amino acid 77 of SEQ ID NO:16 is R; the amino acid in the capsid protein corresponding to amino acid 119 of SEQ ID NO:16 is L; the amino acid in the capsid protein corresponding to amino acid 151 of SEQ ID NO:16 is R; the amino acid in the capsid protein corresponding to amino acid 160 of SEQ ID NO:16 is D; the amino acid in the capsid protein corresponding to amino acid 206 of SEQ ID NO:16 is C; the amino acid in the capsid protein corresponding to amino acid 296 of SEQ ID NO:16 is H; the amino acid in the capsid protein corresponding to amino acid 312 of SEQ ID NO:16 is Q; the amino acid in the capsid protein corresponding to amino acid 346 of SEQ ID NO:16 is A; the amino acid in the capsid protein corresponding to amino acid 464 of SEQ ID NO:16 is N; the amino acid in the capsid protein corresponding to amino acid 468 of SEQ ID NO:16 is S; the amino acid in the capsid protein corresponding to amino acid 501 of SEQ ID NO:16 is I; the amino acid in the capsid protein corresponding to amino acid 505 of SEQ ID NO:16 is R; the amino acid in the capsid protein corresponding to amino acid 590 of SEQ ID NO:16 is R; the amino acid in the capsid protein corresponding to amino acid 626 of SEQ ID NO:16 is G or Y; the amino acid in the capsid protein corresponding to amino acid 681 of SEQ ID NO:16 is M; the amino acid in the capsid protein corresponding to amino acid 687 of SEQ ID NO:16 is R; the amino acid in the capsid protein corresponding to amino acid 690 of SEQ ID NO:16 is K; the amino acid in the capsid protein corresponding to amino acid 706 of SEQ ID NO:16 is C; or, the amino acid in the capsid protein corresponding to amino acid 718 of SEQ ID NO:16 is G. In certain embodiments, the amino acid in the capsid protein corresponding to amino acid 2 of SEQ ID NO:16 is T, and the amino acid in the capsid protein corresponding to amino acid 312 of SEQ ID NO:16 is Q. In certain embodiments, the amino acid in the capsid protein corresponding to amino acid 65 of SEQ ID NO:16 is I, and the amino acid in the capsid protein corresponding to amino acid 626 of SEQ ID NO:16 is Y. In certain embodiments, the amino acid in the capsid protein corresponding to amino acid 77 of SEQ ID NO:16 is R, and the amino acid in the capsid protein corresponding to amino acid 690 of SEQ ID NO:16 is K. In certain embodiments, the amino acid in the capsid protein corresponding to amino acid 119 of SEQ ID NO:16 is L, and the amino acid in the capsid protein corresponding to amino acid 468 of SEQ ID NO:16 is S. In certain embodiments, the amino acid in the capsid protein corresponding to amino acid 626 of SEQ ID NO:16 is G, and the amino acid in the capsid protein corresponding to amino acid 718 of SEQ ID NO:16 is G. In certain embodiments, the amino acid in the capsid protein corresponding to amino acid 296 of SEQ ID NO:16 is H, the amino acid in the capsid protein corresponding to amino acid 464 of SEQ ID NO:16 is N, the amino acid in the capsid protein corresponding to amino acid 505 of SEQ ID NO:16 is R, and the amino acid in the capsid protein corresponding to amino acid 681 of SEQ ID NO:16 is M. In certain embodiments, the amino acid in the capsid protein corresponding to amino acid 505 of SEQ ID NO:16 is R, and the amino acid in the capsid protein corresponding to amino acid 687 of SEQ ID NO:16 is R. In certain embodiments, the amino acid in the capsid protein corresponding to amino acid 346 of SEQ ID NO:16 is A, and the amino acid in the capsid protein corresponding to amino acid 505 of SEQ ID NO:16 is R. In certain embodiments, the amino acid in the capsid protein corresponding to amino acid 501 of SEQ ID NO:16 is I, the amino acid in the capsid protein corresponding to amino acid 505 of SEQ ID NO:16 is R, and the amino acid in the capsid protein corresponding to amino acid 706 of SEQ ID NO:16 is C. In certain embodiments, the capsid protein comprises the amino acid sequence of amino acids 1-736 of any one of SEQ ID NOs:1-15.

In certain embodiments, the instant disclosure provides an rAAV comprising: (a) the AAV capsid comprises one or more of (i) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO:15, (ii) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO:15, and (iii) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO:15; and (b) an rAAV genome comprising any one of the sequences set forth in SEQ ID NOs:117-164.

In certain embodiments, the instant disclosure provides an rAAV comprising: (a) the AAV capsid comprising one or more of (i) a capsid protein comprising the amino acid sequence of amino acids 203-736 of SEQ ID NO:15, (ii) a capsid protein comprising the amino acid sequence of amino acids 138-736 of SEQ ID NO:15, and (iii) a capsid protein comprising the amino acid sequence of amino acids 1-736 of SEQ ID NO:15; and (b) an rAAV genome comprising any one of the sequences set forth in SEQ ID NOs:117-164.

In certain embodiments, the instant disclosure provides an rAAV comprising: (a) the AAV capsid comprising two or more of (i) a capsid protein comprising the amino acid sequence of amino acids 203-736 of SEQ ID NO:15, (ii) a capsid protein comprising the amino acid sequence of amino acids 138-736 of SEQ ID NO:15, and (iii) a capsid protein comprising the amino acid sequence of amino acids 1-736 of SEQ ID NO:15; and (b) an rAAV genome comprising any one of the sequences set forth in SEQ ID NOs:117-164.

In certain embodiments, the instant disclosure provides an rAAV comprising: (a) the AAV capsid comprising (i) a capsid protein comprising the amino acid sequence of amino acids 203-736 of SEQ ID NO:15, (ii) a capsid protein comprising the amino acid sequence of amino acids 138-736 of SEQ ID NO:15, and (iii) a capsid protein comprising the amino acid sequence of amino acids 1-736 of SEQ ID NO:15; and (b) an rAAV genome comprising any one of the sequences set forth in SEQ ID NOs:117-164.

In certain embodiments, the instant disclosure provides an rAAV comprising: (a) the AAV capsid comprises one or more of (i) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO:13, (ii) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO:13, and (iii) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO:13; and (b) an rAAV genome comprising any one of the sequences set forth in SEQ ID NOs:117-164.

In certain embodiments, the instant disclosure provides an rAAV comprising: (a) the AAV capsid comprising one or more of (i) a capsid protein comprising the amino acid sequence of amino acids 203-736 of SEQ ID NO:13, (ii) a capsid protein comprising the amino acid sequence of amino acids 138-736 of SEQ ID NO:13, and (iii) a capsid protein comprising the amino acid sequence of amino acids 1-736 of SEQ ID NO:13; and (b) an rAAV genome comprising any one of the sequences set forth in SEQ ID NOs:117-164.

In certain embodiments, the instant disclosure provides an rAAV comprising: (a) the AAV capsid comprising two or more of (i) a capsid protein comprising the amino acid sequence of amino acids 203-736 of SEQ ID NO:13, (ii) a capsid protein comprising the amino acid sequence of amino acids 138-736 of SEQ ID NO:13, and (iii) a capsid protein comprising the amino acid sequence of amino acids 1-736 of SEQ ID NO:13; and (b) an rAAV genome comprising any one of the sequences set forth in SEQ ID NOs:117-164.

In certain embodiments, the instant disclosure provides an rAAV comprising: (a) the AAV capsid comprising (i) a capsid protein comprising the amino acid sequence of amino acids 203-736 of SEQ ID NO:13, (ii) a capsid protein comprising the amino acid sequence of amino acids 138-736 of SEQ ID NO:13, and (iii) a capsid protein comprising the amino acid sequence of amino acids 1-736 of SEQ ID NO:13; and (b) an rAAV genome comprising any one of the sequences set forth in SEQ ID NOs:117-164.

In another aspect, the instant disclosure provides pharmaceutical compositions comprising a polynucleotide, an rAAV genome, or an rAAV, as disclosed herein, together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current ed., Mack Publishing Co., Easton Pa. 18042, USA; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., 7th ed., Lippincott, Williams, & Wilkins; and Handbook of Pharmaceutical Excipients (2000) A. H. Kibbe et al., 3rd ed. Amer. Pharmaceutical Assoc.

### IV. Methods of Use

In another aspect, the instant disclosure provides methods for expressing a heteromultimeric protein (e.g., an antibody) in a cell. The methods generally comprise introducing into a cell a polynucleotide or rAAV genome, as disclosed herein, or transducing the cell with an rAAV, as disclosed herein.

The methods disclosed herein can be applied to any mammalian cell in which expression of a heteromultimeric protein (e.g., an antibody) is desired, including, but not limited to, hepatocytes (e.g., human hepatocytes).

In certain embodiments, the cell is in a mammalian subject and the polynucleotide, rAAV genome, or rAAV is administered to the subject in an amount effective cause expression of the heteromultimeric protein (e.g., an antibody) in the subject. Accordingly, in certain embodiments, the instant disclosure provides a method for treating a subject having a disease or disorder that would benefit from the expression and secretion of an antibody that specifically binds a therapeutic target, the method generally comprising administering to the subject an effective amount of a polynucleotide, rAAV genome, or rAAV as disclosed herein.

In certain embodiments, the antibody specifically binds to C5 and the disease or disorder is a C5-mediated disease. Accordingly, in certain embodiments, the instant disclosure provides a method of treating a C5-mediated disease in a subject in need thereof, the method comprising administering to the subject an effective amount of a polynucleotide, rAAV genome, or rAAV as disclosed herein. The subject can be a human subject or a rodent subject (e.g., a mouse) into which human liver cells (e.g., human hepatocytes) have been engrafted. Any C5-mediated disease can be treated using the methods disclosed herein. Suitable C5-mediated diseases include, without limitation, paroxysmal nocturnal hemoglobinuria (PNH), neuromyelitis optica spectrum disorder (NMOSD), atypical hemolytic uremic syndrome (aHUS), myasthenia gravis, hematopoietic stem cell transplantation-transplant-associated thrombotic microangiopathy (HSCT-TMA), complement-mediated thrombotic microangiopathy (CM-TMA), Guillain-Barré syndrome, amyotrophic lateral sclerosis (ALS), primary progressive multiple sclerosis (PPMS), multifocal motor neuropathy, antibody-mediated kidney rejection, C3 glomerulopathy, neuromyelitis optica spectrum disorders (NMOSD), age-related macular degeneration (AMD), AQP4 IgG-positive neuromyelitis optica, systemic lupus erythematosus, psoriasis, rheumatoid arthritis (RA), dermatomyositis, idiopathic membranous glomerulopathy, demyelinating neuropathy, complement hyperactivation, angiopathic thrombosis, protein losing enteropathy (CHAPLE) syndrome, geographic atrophy (GA), asthma, proliferative nephritis, and sepsis.

In certain embodiments, the rAAV used in the foregoing methods comprises: (a) the AAV capsid comprises one or more of (i) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO:15, (ii) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO:15, and (iii) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO:15; and (b) an rAAV genome comprising any one of the sequences set forth in SEQ ID NOs:117-164.

In certain embodiments, the rAAV used in the foregoing methods comprises: (a) the AAV capsid comprising one or more of (i) a capsid protein comprising the amino acid sequence of amino acids 203-736 of SEQ ID NO:15, (ii) a capsid protein comprising the amino acid sequence of amino acids 138-736 of SEQ ID NO:15, and (iii) a capsid protein comprising the amino acid sequence of amino acids 1-736 of SEQ ID NO:15; and (b) an rAAV genome comprising any one of the sequences set forth in SEQ ID NOs:117-164.

In certain embodiments, the rAAV used in the foregoing methods comprises: (a) the AAV capsid comprising two or more of (i) a capsid protein comprising the amino acid sequence of amino acids 203-736 of SEQ ID NO:15, (ii) a capsid protein comprising the amino acid sequence of amino acids 138-736 of SEQ ID NO:15, and (iii) a capsid protein comprising the amino acid sequence of amino acids 1-736 of SEQ ID NO:15; and (b) an rAAV genome comprising any one of the sequences set forth in SEQ ID NOs:117-164.

In certain embodiments, the rAAV used in the foregoing methods comprises: (a) the AAV capsid comprising (i) a capsid protein comprising the amino acid sequence of amino acids 203-736 of SEQ ID NO:15, (ii) a capsid protein comprising the amino acid sequence of amino acids 138-736 of SEQ ID NO:15, and (iii) a capsid protein comprising the amino acid sequence of amino acids 1-736 of SEQ ID NO:15; and (b) an rAAV genome comprising any one of the sequences set forth in SEQ ID NOs:117-164.

In certain embodiments, the instant disclosure provides an rAAV comprising: (a) the AAV capsid comprises one or more of (i) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO:13, (ii) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO:13, and (iii) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO:13; and (b) an rAAV genome comprising any one of the sequences set forth in SEQ ID NOs:117-164.

In certain embodiments, the rAAV used in the foregoing methods comprises: (a) the AAV capsid comprising one or more of (i) a capsid protein comprising the amino acid sequence of amino acids 203-736 of SEQ ID NO:13, (ii) a capsid protein comprising the amino acid sequence of amino acids 138-736 of SEQ ID NO:13, and (iii) a capsid protein comprising the amino acid sequence of amino acids 1-736 of SEQ ID NO:13; and (b) an rAAV genome comprising any one of the sequences set forth in SEQ ID NOs:117-164.

In certain embodiments, the rAAV used in the foregoing methods comprises: (a) the AAV capsid comprising two or more of (i) a capsid protein comprising the amino acid sequence of amino acids 203-736 of SEQ ID NO:13, (ii) a capsid protein comprising the amino acid sequence of amino acids 138-736 of SEQ ID NO:13, and (iii) a capsid protein comprising the amino acid sequence of amino acids 1-736 of SEQ ID NO:13; and (b) an rAAV genome comprising any one of the sequences set forth in SEQ ID NOs:117-164.

In certain embodiments, the rAAV used in the foregoing methods comprises: (a) the AAV capsid comprising (i) a capsid protein comprising the amino acid sequence of amino acids 203-736 of SEQ ID NO:13, (ii) a capsid protein comprising the amino acid sequence of amino acids 138-736 of SEQ ID NO:13, and (iii) a capsid protein comprising the amino acid sequence of amino acids 1-736 of SEQ ID NO:13; and (b) an rAAV genome comprising any one of the sequences set forth in SEQ ID NOs:117-164.

An AAV composition disclosed herein can be administered to a subject by any appropriate route including, without limitation, intravenous, intraperitoneal, subcutaneous, intramuscular, intranasal, topical, or intradermal routes. In certain embodiments, the composition is formulated for administration via intravenous injection or subcutaneous injection.

### V. AAV Packaging Systems

In another aspect, the instant disclosure provides packaging systems for recombinant preparation of a recombinant adeno-associated virus (rAAV) disclosed herein. Such packaging systems generally comprise: a first nucleotide encoding one or more AAV Rep proteins; a second nucleotide encoding a capsid protein of any of the AAVs as disclosed herein; and a third nucleotide sequence comprising any of the rAAV genomes as disclosed herein, wherein the packaging system is operative in a cell for enclosing the rAAV genome in the capsid to form the AAV.

In certain embodiments, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.).

Any AAV Rep protein can be employed in the packaging systems disclosed herein. In certain embodiments of the packaging system, the Rep nucleotide sequence encodes an AAV2 Rep protein. Suitable AAV2 Rep proteins include, without limitation, Rep 78/68 or Rep 68/52.

In certain embodiments of the packaging system, the packaging system further comprises a fourth nucleotide sequence comprising one or more helper virus genes. In certain embodiments of the packaging system, the packaging system further comprises a third vector, e.g., a helper virus vector, comprising the fourth nucleotide sequence comprising the one or more helper virus genes. The third vector may be an independent third vector, integral with the first vector, or integral with the second vector.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4, and VA. In certain embodiments of the packaging system, where the helper virus is HSV, the HSV genome comprises one or more of HSV genes selected from the group consisting of UL5/8/52, ICPO, ICP4, ICP22, and UL30/UL42.

In certain embodiments of the packaging system, the first, second, and/or third vector are contained within one or more plasmids. In certain embodiments, the first vector and the third vector are contained within a first plasmid. In certain embodiments the second vector and the third vector are contained within a second plasmid.

In certain embodiments of the packaging system, the first, second, and/or third vector are contained within one or more recombinant helper viruses. In certain embodiments, the first vector and the third vector are contained within a recombinant helper virus. In certain embodiments, the second vector and the third vector are contained within a recombinant helper virus.

In a further aspect, the disclosure provides a method for recombinant preparation of an AAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g., with one or more recombinant helper viruses, such as a adenovirus, poxvirus (such as vaccinia virus), herpes virus (including HSV, cytomegalovirus, or baculovirus, containing a first, and a second, and optionally a third vector as described herein)), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more AAV capsid proteins as described herein, and with an rAAV genome as described herein being delivered in the form of a plasmid or a recombinant helper virus).

Accordingly, the instant disclosure provides a packaging system for preparation of a recombinant AAV (rAAV), wherein the packaging system comprises a first nucleotide sequence encoding one or more AAV Rep proteins; a second nucleotide sequence encoding a capsid protein of any one of the AAVs described herein; a third nucleotide sequence comprising an rAAV genome sequence of any one of the AAVs described herein; and optionally a fourth nucleotide sequence comprising one or more helper virus genes.

### VI. Examples

The following examples demonstrate the efficient and sustained expression of antibodies (e.g., anti-C5 antibodies) in a subject using rAAV vectors comprising the novel bidirectional dual promoter expression systems disclosed herein. These examples are offered by way of illustration, and not by way of limitation.

### Example 1: Evaluation of Performance of Single and Dual Promoter Vector Designs

Three different anti-C5 antibody-expressing rAAV vectors were designed (Ab-02, Ab-03, and Ab-04), and the ability of these vectors to effect efficient and sustained expression of anti-C5 antibody in the serum of NOD-SCID mice was determined.

Vector Ab-02 uses a single liver-specific promoter to drive expression of the antibody heavy chain and light chain. Specifically, Ab02, comprises, from 5′ to 3′, the following genetic elements: a transcriptional regulatory element comprising the liver-specific LP1 promoter; a coding sequence encoding a human IgG2 signal sequence operably linked to an anti-C5 antibody heavy chain; a nucleic acid sequence encoding a 2A ribosomal skipping peptide; a coding sequence encoding an Igκ signal sequence operably linked to an anti-C5 antibody light chain; and an SV40 late polyadenylation sequence. The nucleic sequence of this vector is set forth in SEQ ID NO:73.

Vector Ab-03 uses two tandem liver-specific promoters to drive expression of the antibody heavy chain and light chain. Specifically, Ab03 comprises, from 5′ to 3′, the following genetic elements: a transcriptional regulatory element comprising the liver-specific LP1 promoter; a coding sequence encoding a human IgG2 signal sequence operably linked to an anti-C5 antibody heavy chain; a bovine growth hormone polyadenylation signal; a transcriptional regulatory element comprising the liver-specific DnG promoter; a coding sequence encoding an Igκ signal sequence operably linked to an anti-C5 antibody light chain; and an SV40 late polyadenylation sequence. The nucleic sequence of this vector is set forth in SEQ ID NO:74.

Vector Ab-04 uses a liver-specific bidirectional dual promoter system to drive expression of the antibody heavy chain and light chain. Specifically, Ab04 comprises from 5′ to 3′, the following genetic elements: a reverse complement bovine growth hormone polyadenylation signal; a reverse complement coding sequence encoding a human IgG2 signal sequence operably linked to an anti-C5 antibody heavy chain; a reverse complement transcriptional regulatory element comprising the liver-specific LP1 promoter; a rabbit beta globin polyadenylation sequence; a transcriptional regulatory element comprising the liver-specific DnG promoter; a coding sequence encoding an Igκ signal sequence operably linked to an anti-C5 antibody light chain; and an SV40 late polyadenylation sequence. The nucleic sequence of this vector is set forth in SEQ ID NO:75.

Each of the aforementioned constructs were packaged into both AAVHSC15 and AAVHSC17 capsids and administered to male NOD-SCID mice at dose of 1e13 vg/kg. Serum samples were collected after 1, 3, 5, 7, 9, 13, and 16 weeks (3 mice per time point), and antibody levels were measured using a human IgG-specific ELISA (the SIMPLESTEP ELISA® kit from Abcam), according to the manufacturer's instructions.

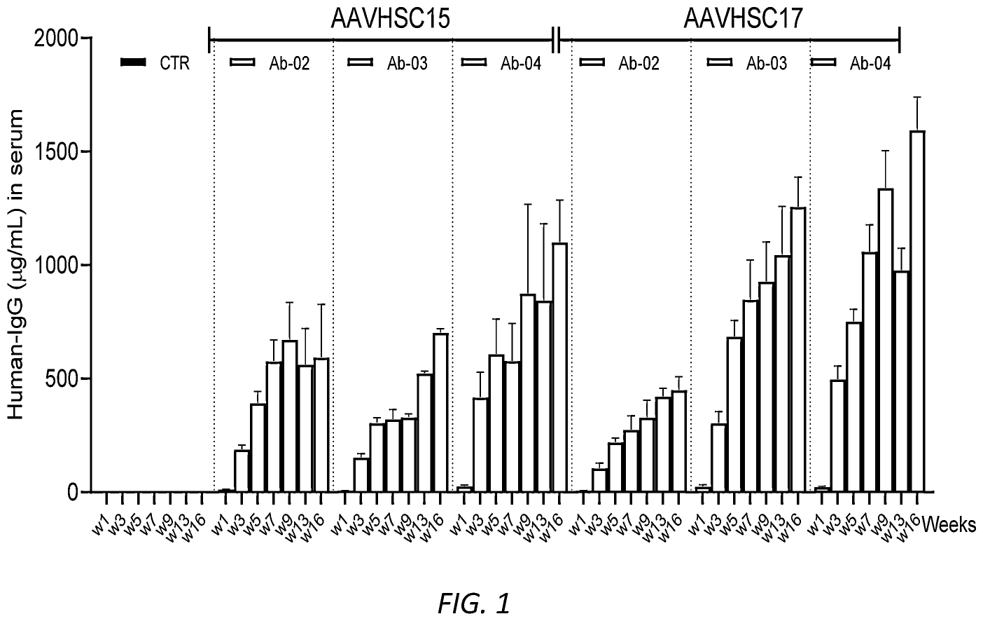

As shown in FIG. 1, the bidirectional dual promoter construct (Ab04) packaged in either an AAVHSC15 or an AAVHSC17 capsid resulted in higher serum IgG levels than either the single promoter construct (Ab04) or the tandem dual promoter construct (Ab03).

### Example 2: Bidirectional Dual Promoter Vectors

In view of the superior serum antibody expression levels exhibited by the bidirectional dual promoter vector Ab-04 relative to vectors Ab-02, and Ab-03, 25 alternative bidirectional dual promoter vectors were designed in an effort to further optimize antibody expression and reduce immunogenicity. The sequences of the major genetic elements present in these vectors are set forth in Table 1 herein. Each of these vectors comprised from 5′ to 3′: a reverse complement first polyadenylation sequence; a reverse complement coding sequence for an antibody light chain; an intron element; a reverse complement hAAT promoter sequence; an hHCR1 sequence or complement reverse hHCR1 sequence; a TTR promoter sequence; an intron element; a coding sequence for an antibody heavy chain; and a second polyadenylation sequence. In certain vectors an rBG-pA sequence was interposed between the hHCR1 sequence and the TTR promoter sequence.

### Example 3: In Vivo Expression of Humanized Anti-C5 Antibodies in Mice

The anti-C5 antibody expression and functional activity of 19 of the vector designs set forth in Table 1 were evaluated in vivo in NOD-SCID mice (NOD.CB-17-Prkdcscid/J mice; The Jackson Laboratory, Bar Harbor, ME, USA). An immunocompromised model was selected because humanized antibodies have the potential to elicit anti-drug antibodies in mice. NOD-SCID mice were selected because they are deficient for endogenous murine C5, as they carry the HCO allele. The absence of endogenous C5 allowed for the precise measurement of the functional activity of the anti-C5 antibody using an ex vivo hemolysis assay.

In order to evaluate whether sufficiently high levels of anti-C5 antibodies can be expressed in the serum of an organism, NOD-SCID mice were dosed intravenously at 1e13 vg/kg with each of vectors E1-100, E40, E41, E42, E43, E44, E45, E46, E47, E48, E49, E50, E51, E52, E53, E54, E55, E56, and E57 (each packaged in an AAVHSC13 capsid), or with formulation buffer control (FB). Serum samples were collected at 1, 3, 5, 7, 9, 11, and 12 weeks (5 mice per time point), and antibody levels were measured using a human IgG-specific ELISA, as described in Example 1. Vector genome copy number in the liver of these mice was determined after 12 weeks by droplet digital PCR (ddPCR) using a primer probe set common to all 19 vectors and not present in the murine genome.

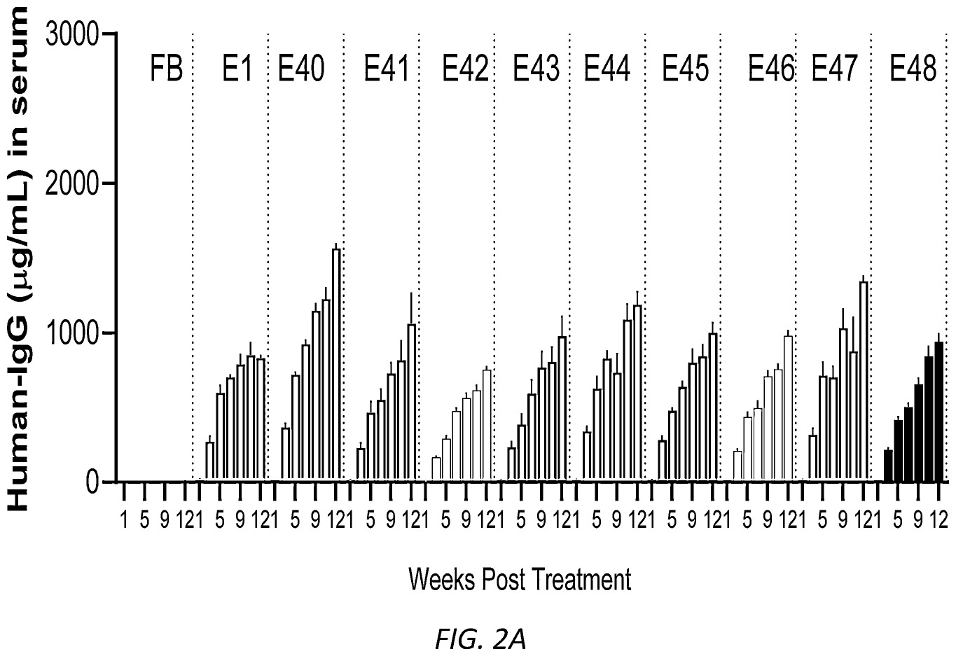

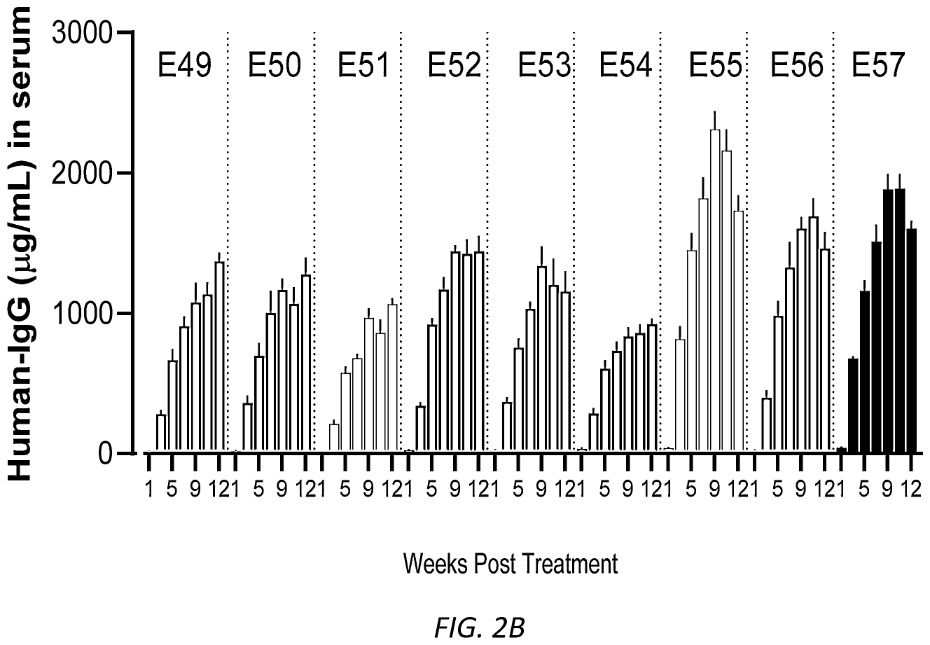

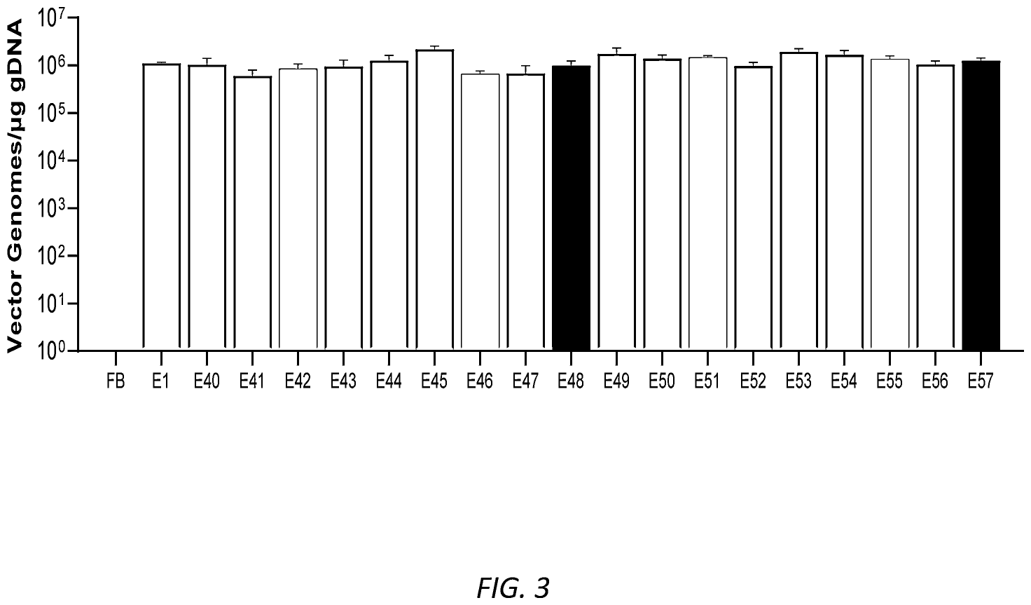

As shown in FIG. 2A and FIG. 2B, antibody levels were detectable 1 week post treatment. All vectors resulted in elevated and sustained levels of anti-C5 antibodies in serum over time. Moreover, many constructs showed improved antibody levels as compared to reference construct E1-100, with some constructs achieving milligram concentrations of the antibody. As shown in FIG. 3, the number of vector genomes per μg of genome DNA in the livers of these mice were comparable across all groups.

### Example 4: Effect of Vectorized Anti-C5 Antibody Expression on Ex Vivo Hemolysis

Paroxysmal nocturnal hemoglobinuria (PNH) is characterized by destruction of red blood cells (hemolytic anemia), blood clots (thrombosis), and impaired bone marrow function. To determine if the anti-C5 antibodies expressed from the vectors described herein would be effective at reducing hemolytic anemia, the functional activity of transduced anti-C5 antibody was evaluated using an ex vivo hemolysis assay initiated by the classical complement pathway at week 1 and week 3 for all constructs, and at week 12 for selected constructs.

Briefly, human serum containing human C5 was mixed with serum obtained from the NOD-SCID mice that had been treated with AAVHSC13-packaged C5Ab. The mouse serum was collected at 1 week, 3 weeks, and 12 weeks following treatment. The human serum and mouse serum were mixed with activated sheep red blood cells (RBCs). The ex vivo hemolysis assay for NOD-SCID studies measured inhibition of complement-mediated lysis of the activated sheep RBCs induced by normal human serum that is combined in equal parts with either NOD-SCID serum from vector-treated animals or formulation buffer-treated controls. Specifically, in a 96-well V-bottom plate, Gelatin Veronal buffer (GVBS, Sigma, Cat #G6514) was mixed with mouse serum in each well (with and without EDTA). A solution of 10% Normal Human Serum (NHS, Sigma, Cat #H4522) was then added to all wells. The plate was then incubated at 37° C. for 30 minutes. An aliquot of 1 mL of antibody-sensitized sheep RBCs (Complement Technology, Inc., Catalog Numbers: B200, B201, and B202) was then added to each well and shaken for about 30 minutes. The plate was then centrifuged at 1000 g for 5 minutes. The supernatant was moved to a new plate and read at 412 nm and 615 nm. The 615 nm value was then subtracted from 412 nm value to obtain the final reading. In all reported % hemolysis values, % hemolysis of all samples, including the formulation buffer-only or AAVHSC-treated samples, is reported after normalizing with a 100% red blood cell lysis control.

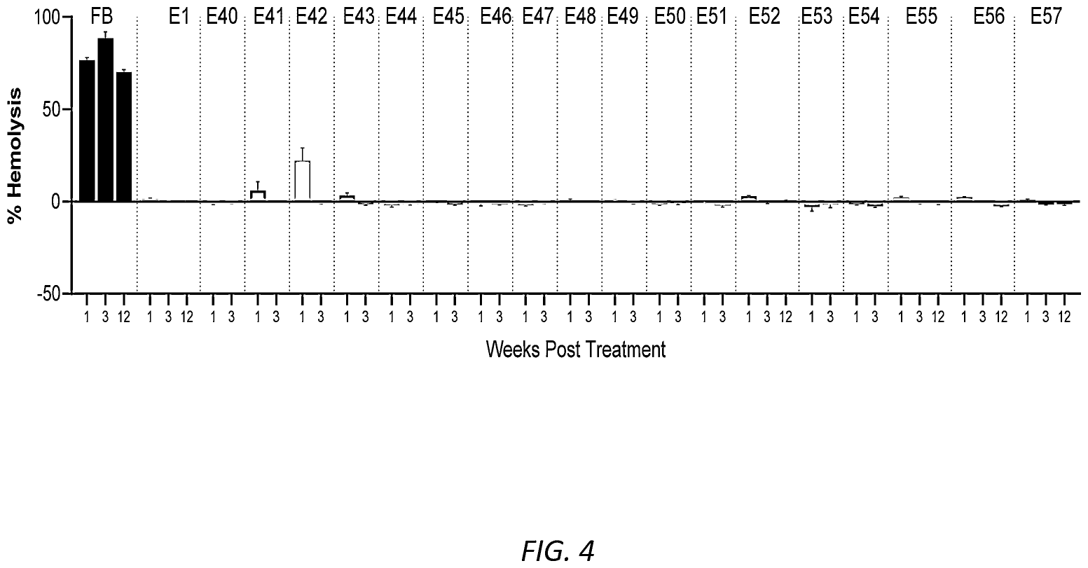

As shown in FIG. 4, RBC hemolysis was abrogated by serum from all rAAV-treated animals as early as 1 week post-administration, and no hemolysis was observed after 3 weeks.

### Example 5: Vector Optimization

In order to maximize sustained expression of anti-C5 antibody in serum, the bidirectional dual promoter constructs disclosed herein were optimized relative to the design used in the Ab04 construct described in Example 1. Nineteen constructs where evaluated, each with modifications to vector elements in order to optimize performance. These optimizations included: lengths of the TTR and hAAT promoter sequences; length and directionality of the hHCR1 sequence; use of intron elements; use of polyadenylation sequences; and CpG content in coding and non-coding regions. Changes that led to substantial improvements in vector performance were identified, both in terms of kinetics of antibody expression, as well as overall steady-state antibody levels in serum. The reference vector, E1-100, contains the same bidirectional dual promoter design as Ab04, and was used as the reference vector for all other vector designs. E1-100 robustly expressed an anti-C5 mAb when dosed at 1e13 vg/kg (>800 μg/mL). All assays were performed as described in the preceding Examples.

### (A) Effect of Back-to-Back Placement of Liver Specific Promoters on the Need for Separate Enhancer Elements in the TTR Promoter

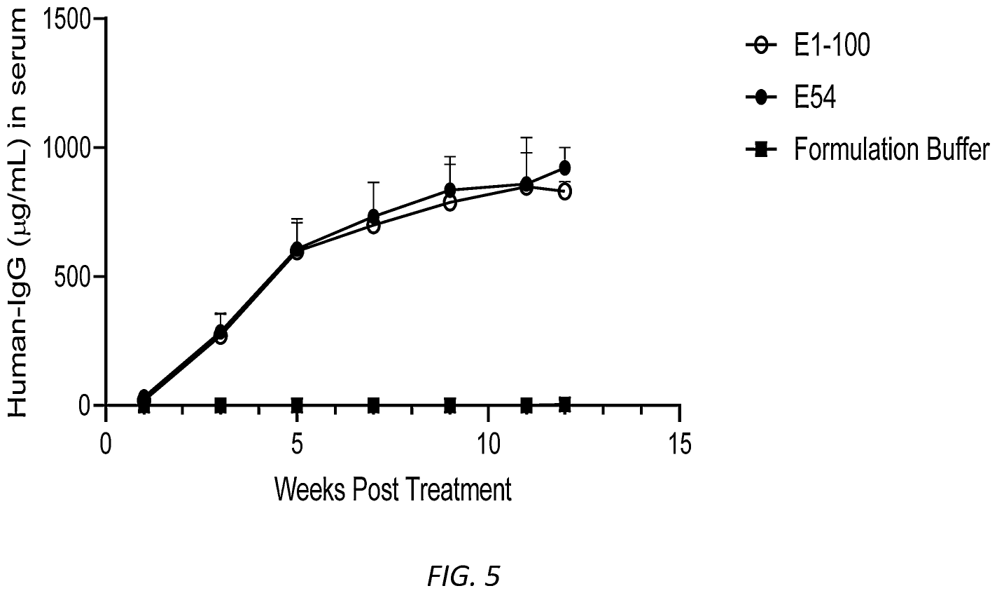

To determine whether each of the two promoters in the bidirectional dual promoter unit required its own separate enhancer element, vector E54 was compared to E1-100. Relative to E1-100, the TTR promoter sequence in E54 lacks an enhancer element and has an increased length (202 bases compared to 170 bases in E1-100). As shown in FIG. 5, the bidirectional dual promoter unit in the E54 vector resulted in similar serum IgG expression as the E1-100 vector. This result demonstrates that a separate enhancer element for the TTR promoter is not required in the context of a bidirectional dual promoter.

### (B) Effect of CpG-Free Payload Codon Optimization on IgG Expression

Traditional codon optimizations generally employ the most commonly used codon and bicodon sequences to drive high transgene expression, and frequently yield sequences with high CpG content. In the case of vectors E1-100 and E54, the antibody heavy chain coding sequences contain 55 CpGs, while the light chain coding sequences contain 33 CpGs. High CpG content has been shown in preclinical and clinical trials to induce immune-mediated TLR9 activation, which can lead to loss of durability in transgene expression. However, removal of CpGs from transgenes often requires the use of less frequently used codons, which has the potential to compromise expression levels.

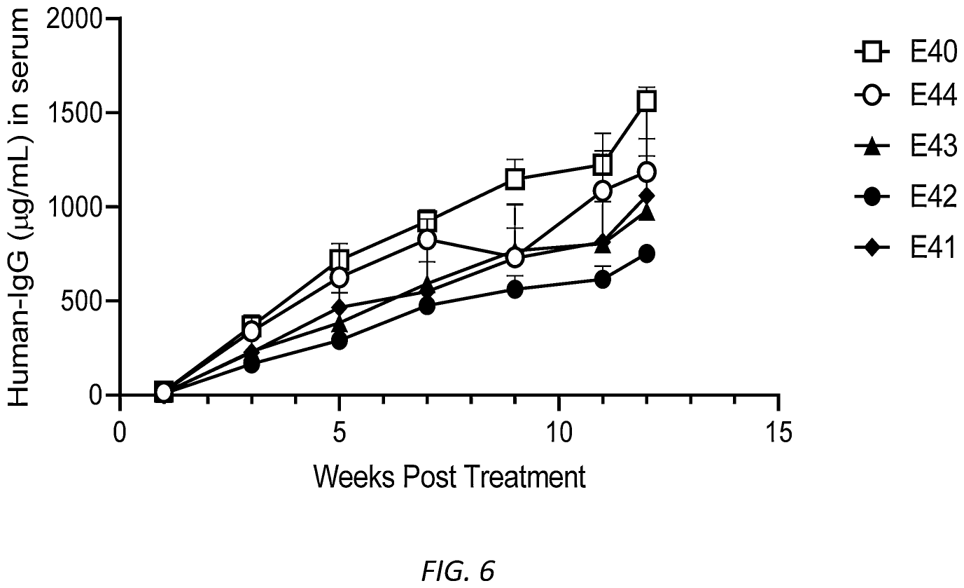

In order to reduce CpGs while not jeopardizing transgene expression, codon optimization was performed, and expression of coding sequences was evaluated using a series of five constructs. Each of the constructs differed in the codon optimization used for the antibody coding sequences and signal peptide coding sequences, but were otherwise identical in vector design and vector elements. These constructs included vectors E40, E41, E42, E43, and E44. The effect of coding sequence codon optimization on anti-C5 antibody expression was evaluated over time in NOD-SCID mice. As shown in FIG. 6, vector constructs optimized for reduced CpG content yielded a two-fold increase in anti-C5 antibody expression as determined by IgG expression. Notably, the CpG-free codon-optimized E40 construct yielded the highest IgG serum levels compared to the E41, E42, and E43 vector constructs at week 12.

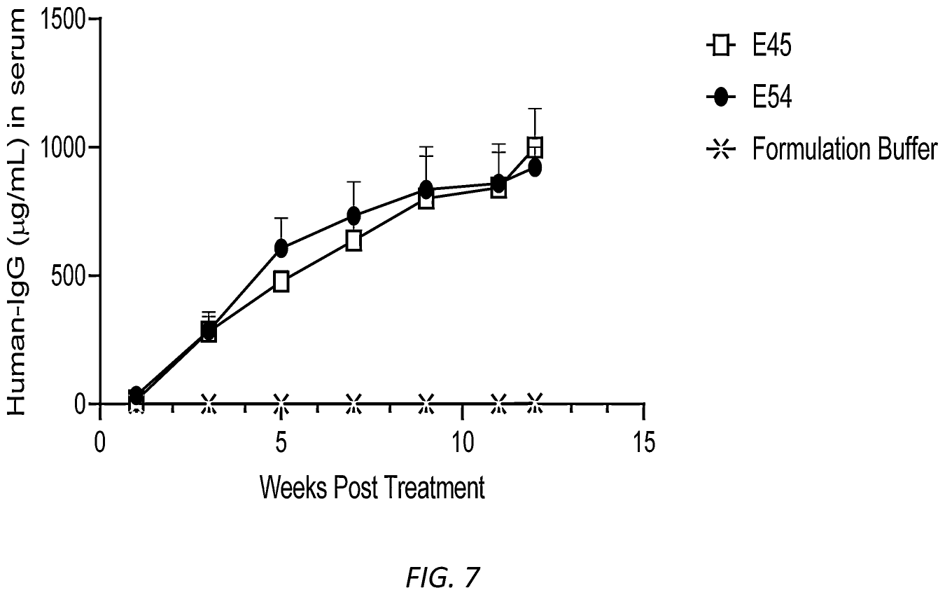

In the same study, IgG expression of a CpG-free optimized codon vector (E45) was compared to that of a construct with high CpG-content that was selected solely for high IgG expression (E54). As shown in FIG. 7, vector E45, which contains the same vector design as E54, but has the transgene codon optimizations from vector E40, performed equally well to vector E54. These results indicate that, surprisingly, high CpG content was not necessary to drive strong antibody expression.

(C) Effect of hAAT Promoter Size and Sequence and hHCR1 Enhancer Size and Directionality on IgG Expression

In order to evaluate the impact of hAAT promoter size, hHCR1 enhancer size and hHCR1 enhancer directionality on IgG expression, transgene expression in the E40, E45, E46, E47, and E48 vectors was evaluated.

The effect of the hAAT promoter length was determined by comparing: the E40, E45, and E46 vectors, each of which have a 255 base pair-containing hAAT promoter sequence; and the E47 and E48 vectors, each of which have a novel 180 base pair-containing hAAT promoter sequence.

The effect of the hHCR1 enhancer length was determined by comparing: the E45 vector which contains a 192 base pair hHCR1 enhancer sequence; and the E40, E46, E47, and E48 vectors, each of which have a 314 base pair-containing hHCR1 enhancer sequence.

The effect of directionality was determined by comparing: the E40 and E47 vectors (each of which have the hHCR1 enhancer sequence in the forward orientation); and the E45, E46, and E48 vectors (each of which have the hHCR1 enhancer sequence in the reverse orientation). All five of these constructs contained the same intron elements, polyadenylation sequences, and codon optimized coding sequences.

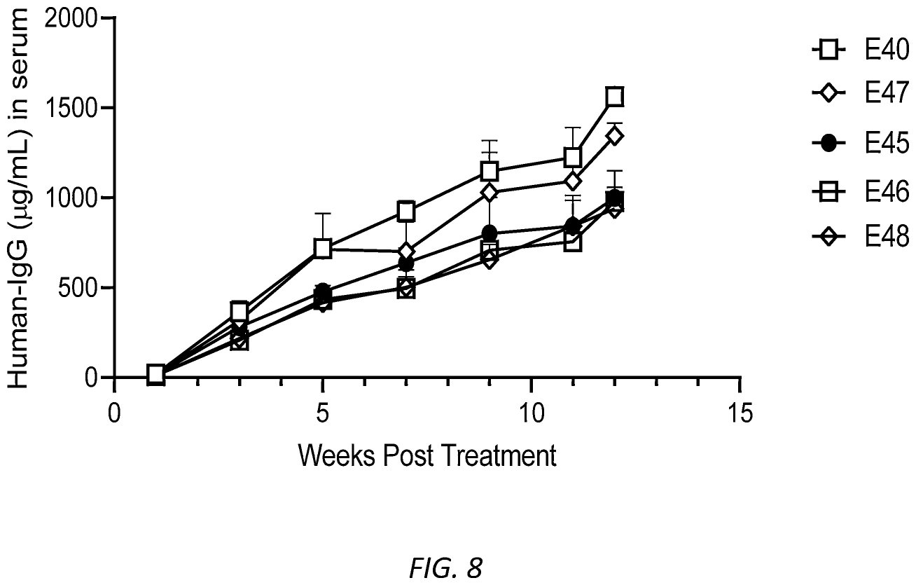

As shown in FIG. 8, each of vectors E45, E46, and E48 exhibited a similar level of IgG expression in mouse serum, demonstrating that the novel 180 base minimal hAAT promoter was equally effective at supporting antibody expression as the longer 255 base hAAT sequence. The data in FIG. 8 also demonstrated that: the 192 base hHCR1 enhancer sequence enhanced expression to a similar extent as the 314 base hHCR1 enhancer sequence; the hHCR1 enhancer sequence was functional in both the forward and reverse orientations, with the forward orientation causing more IgG expression than the reverse orientation.

### (D) Effect of a Novel Chimeric Intron for the TTR Promoter on IgG Expression

The effect on transgene expression of placing an intron downstream of the TTR promoter sequence was evaluated. An MVM intron as well as a chimeric beta globin intron were introduced, and transgene expression was evaluated. The chimeric beta globin intron used herein had previously been identified in 8-week studies by Applicant to yield improvements in transgene expression. However, the sequence contains a high number of CpGs within the intron, stuffer elements, or adjacent restriction enzymes (10 CpGs total). Accordingly, using in-silico publicly available software to predict splicing, the promoter sequence was optimized to remove most of the CpGs within the intron and stuffer sequences (only 2 CpGs remained).

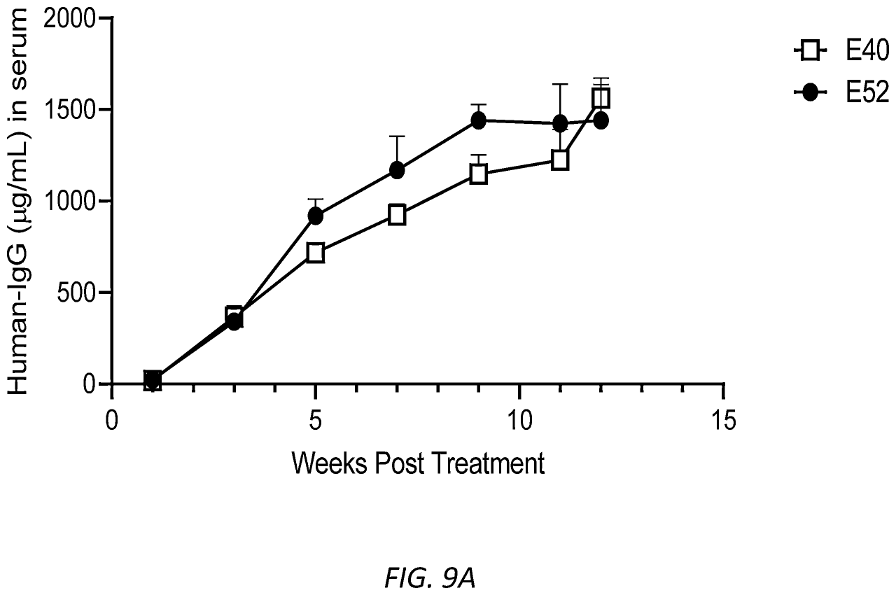

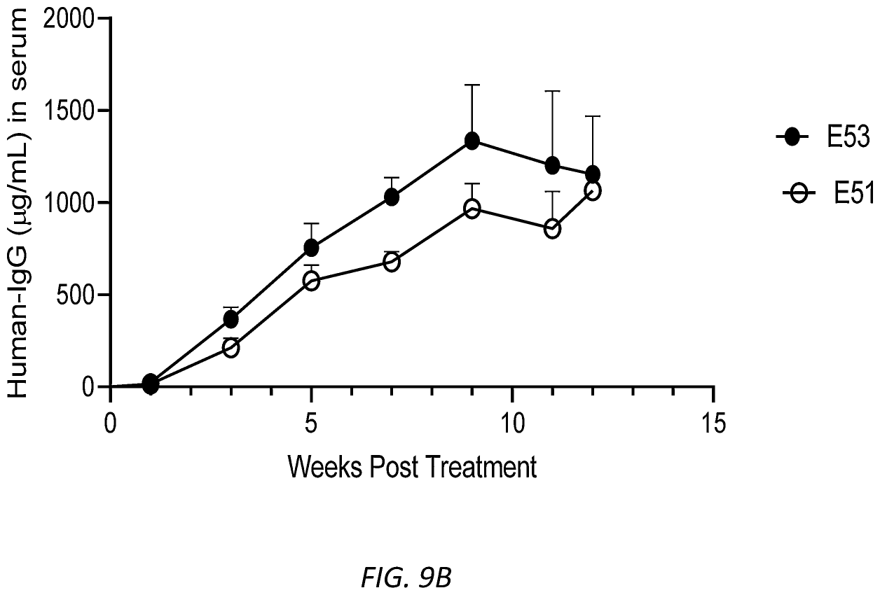

Pairs of constructs were evaluated that had identical vector designs except that they differed in whether they contain the MVM intron (e.g., vectors E40 and E51) or the novel chimeric beta globin intron with reduced CpGs (e.g., vectors E52 and E53), to drive antibody light chain expression. As shown in FIG. 9A and FIG. 9B, the vector constructs with reduced CpGs produced increased antibody expression at earlier time points and reached steady state levels of antibody expression in less time than constructs containing the MVM intron (vectors E40 vs E51).

### (E) Effect of Further Reduction of CpGs on IgG Expression

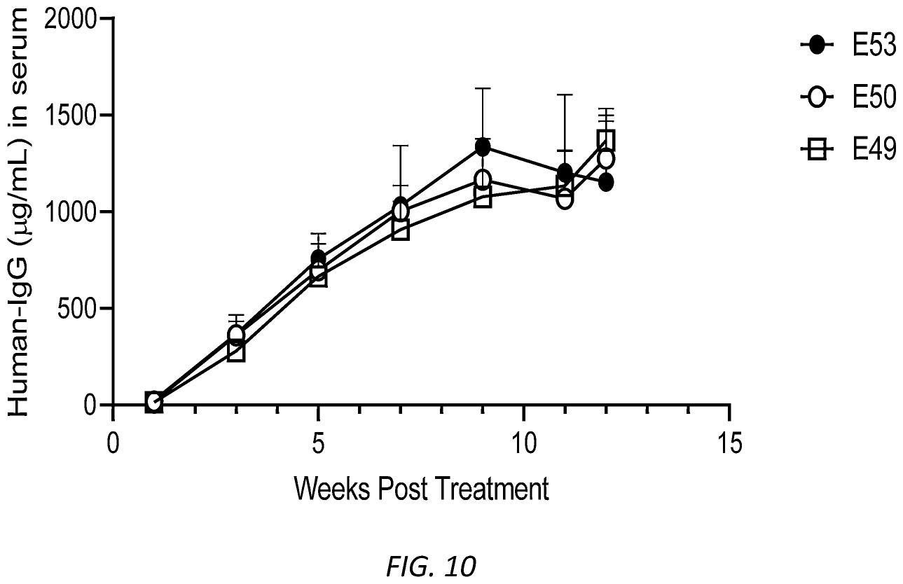

In order to evaluate whether further changes in the number of CpGs within non-coding regions of the vector design had an effect on transgene expression, the number of CpGs within the intron and the promoter regions was further reduced, and IgG levels were measured. Vectors containing the chimeric beta globin intron were used, including a novel chimeric beta globin intron optimized to eliminate CpG content entirely. The E53 vector (containing a chimeric beta globin intron with 2 CpGs) was compared to the E50 vector (containing the novel chimeric beta globin intron with 0 CpGs). As shown in FIG. 10, the removal of the remaining 2 CpGs from the chimeric beta globin intron in E50 was well tolerated and did not compromise IgG expression.

In addition, the effect of removing CpGs within other non-coding regions on transgene expression was evaluated in order to determine whether replacing the remaining CpGs by conserved substitutions disrupted transcription binding sites. IgG expression using the E50 vector (containing all 10 CpGs), was compared to that of the E49 vector (containing 6 CpGs). As shown in FIG. 10, removal of 4 CpGs within the hAAT promoter was well tolerated and did not impact expression of IgG.

(F) Effect of Polyadenylation Sequence Introduction into the Bidirectional Dual Promoter Unit on IgG Expression

The effect of placing a rabbit beta globin polyadenylation (rBG-pA) sequence between the two promoters in the bidirectional dual promoter unit disclosed herein was evaluated. It was found that the rate of IgG expression as well as the steady state IgG expression levels were significantly increased in constructs containing an rBG-pA within the bidirectional promoter unit.

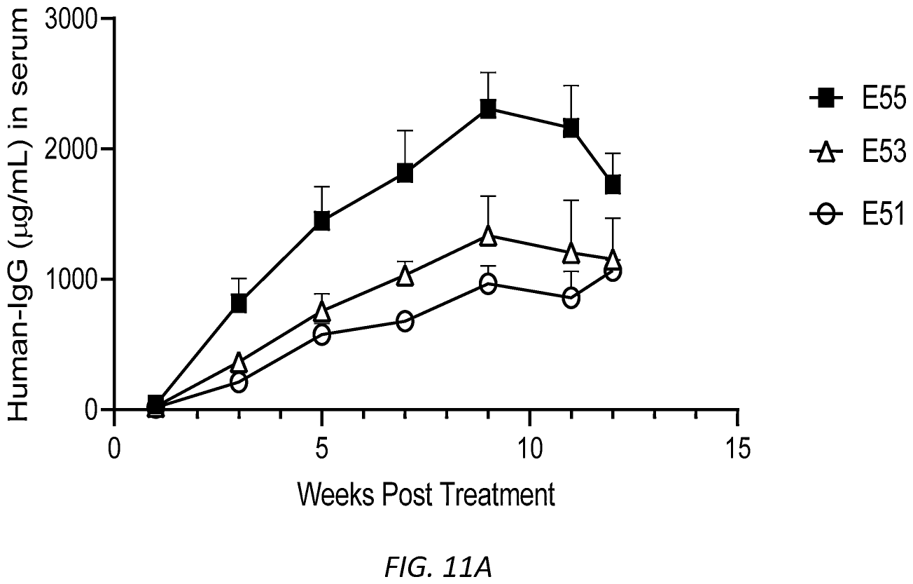

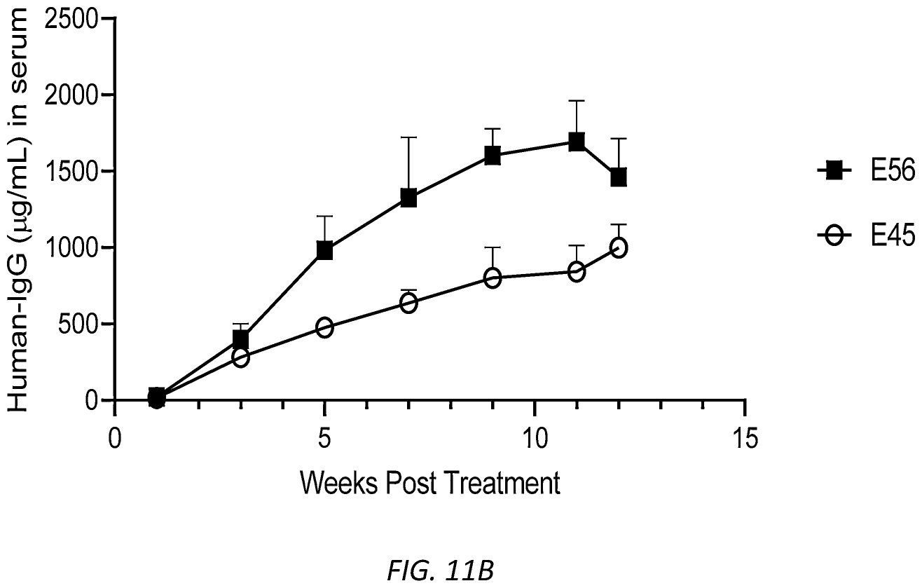

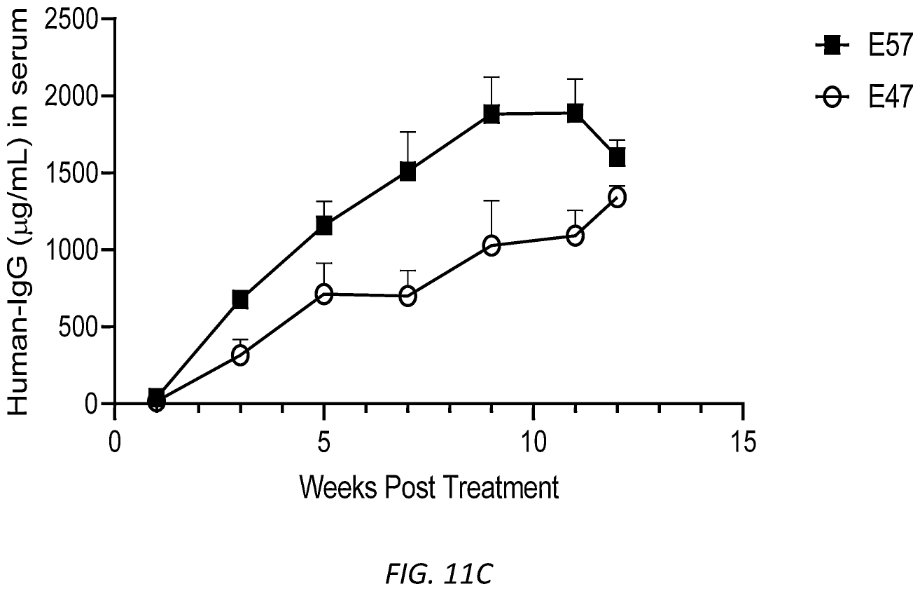

The combined effect of substituting the MVM intron for the novel CpG-reduced chimeric intron (described above), and the addition of the rBG-pA stuffer was demonstrated by comparing three pairs of vectors (E51 vs E55; see FIG. 11A), (E45 vs E56; see FIG. 11B), and (E47 vs E57; see FIG. 11C). Each of these vector pairs contained different bidirectional dual promoter designs, but within each pair, the other vector elements are identical. In FIG. 11A, FIG. 11B, and FIG. 11C, the IgG expression levels for vectors with the MVM intron are represented in white circles (E51, E45, E47), vectors containing the CpG-reduced chimeric beta globin intron are shown in gray triangles (E53), and vectors containing the rBG-pA combined with the CpG-reduced chimeric beta globin intron are shown in black squares (E55, E56, and E57).

As shown in FIG. 11A, FIG. 11B, and FIG. 11C, the addition of a rBG-pA 5′ of the TTR promoter resulted in a further enhancement in the rate of serum IgG production, leading to increased steady state IgG levels.

### (G) Effect of Polyadenylation Sequences on IgG Expression

Polyadenylation sequences can regulate mRNA stability and overall antibody expression levels. Accordingly, the effect of polyadenylation sequences on overall IgG expression was evaluated by comparing vectors containing a novel synthetic polyadenylation sequence (Syn-pA) to vectors containing a bovine growth hormone polyadenylation sequence (bGH-pA). Specifically, the E40 and E52 vectors (containing bGH-pA), and the E51 and E53 vectors (containing Syn-pA) were compared. Each of these pairs of vectors differed only in the polyadenylation sequence for the heavy chain.

Within the same experiment, the effect of the intron used for the TTR promoter on IgG expression was also assessed. The E40 and E51 vectors (containing an MVM intron) were compared to the E52 and E53 vectors (containing a modified chimeric beta globin intron with reduced CpGs).

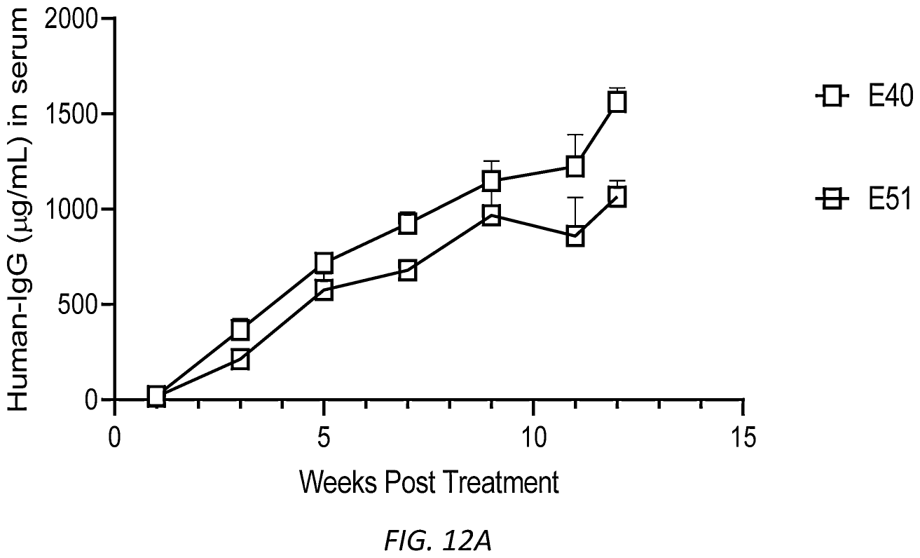

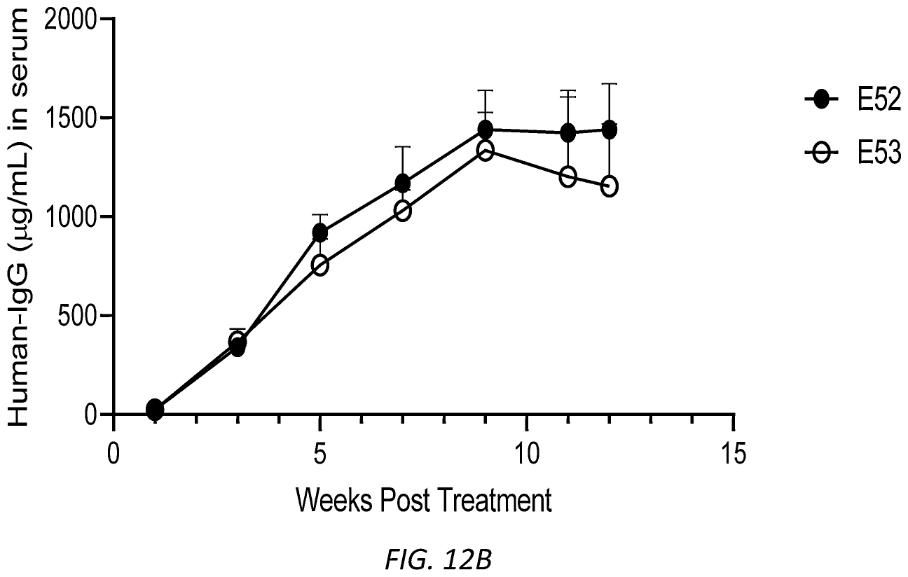

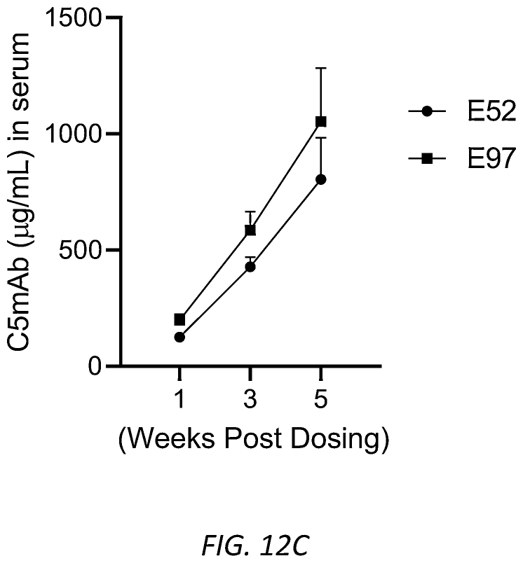

As shown in FIG. 12A and FIG. 12B, there was a statistically significant increase in IgG expression using vectors with the bGH-pA as compared to expression using the Syn-pA for the constructs containing the MVM intron (the E40 and E51 vectors). However, the Syn-pA and bGH-pA performed similarly in constructs containing a modified chimeric beta globin intron with reduced CpGs 5′ of the TTR promoter (the E52 and E53 vectors).

In a separate experiment, the effect of removing CpGs within polyadenylation sequences on transgene expression was evaluated. C57BL/6J mice were dosed intravenously at 3e12 vg/kg with vectors E52 or E97 (each packaged in an AAVHSC13 capsid). The E97 vector is substantially similar to the E52 vector except that E97 contains a novel BGH polyadenylation sequence with 2 CpGs removed (see Table 1). Serum samples were collected at 1, 3, and 5 weeks post-dosing (5 mice per time point), and antibody levels were measured using a human IgG-specific ELISA. As shown in FIG. 12C, removal of 2 CpGs within the BGH polyadenylation sequence was well tolerated and did not impact expression of the anti-C5 antibody.

### Example 6: Durability and Dose Response Study of an Optimized Vector

In order to investigate the relationship between the dosage administered of a bidirectional dual promoter vector and anti-C5 antibody expression level and functional activity, NOD-SCID mice were dosed intravenously at 3e11, 3e12, 1e13, 3e13, and 1e14 vg/kg with vector E55 (packaged in an AAVHSC13 capsid), or with formulation buffer control (vehicle). 5 male mice were dosed in each group. Serum samples were collected from each mouse at 1, 3, 5, 7, 9, 11, 13, and 15 weeks, and antibody levels were measured using a human IgG-specific ELISA, as described in Example 1. Terminal serum samples were collected from each mouse at 4 and 16 weeks.

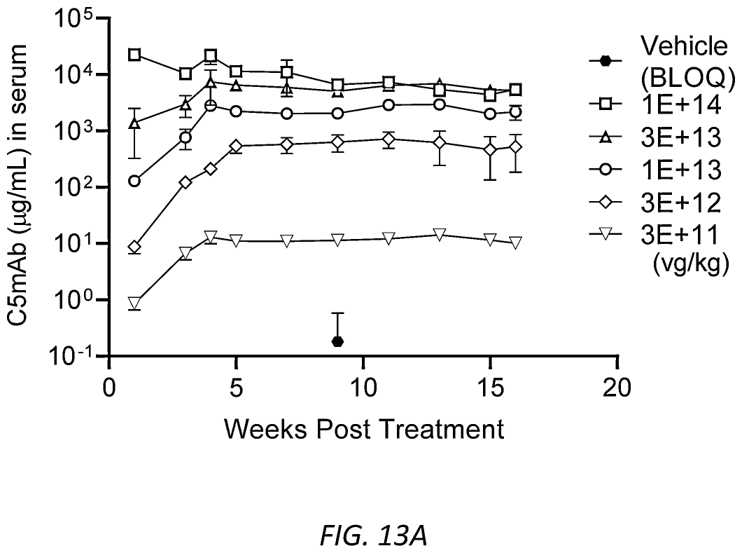

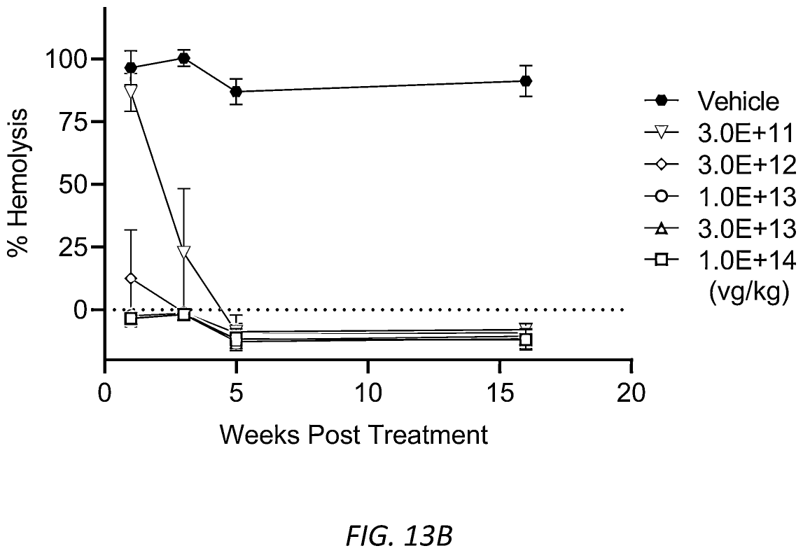

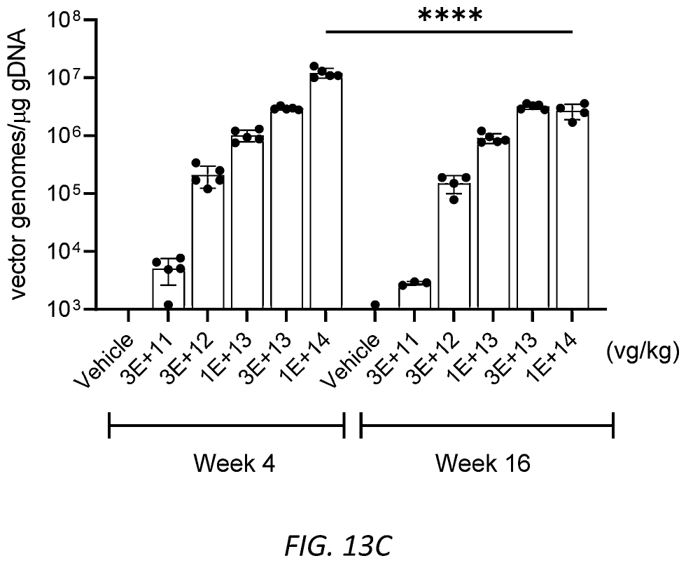

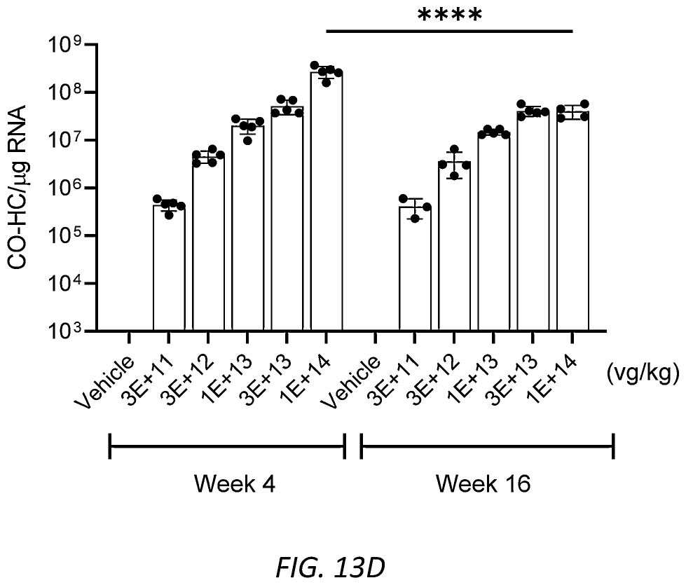

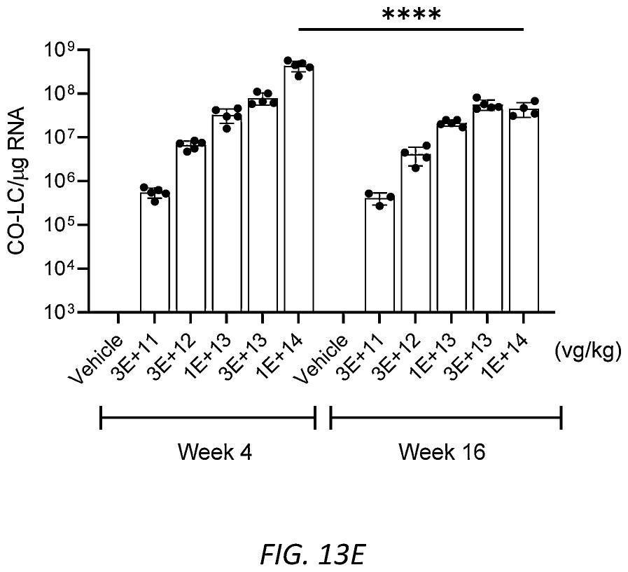

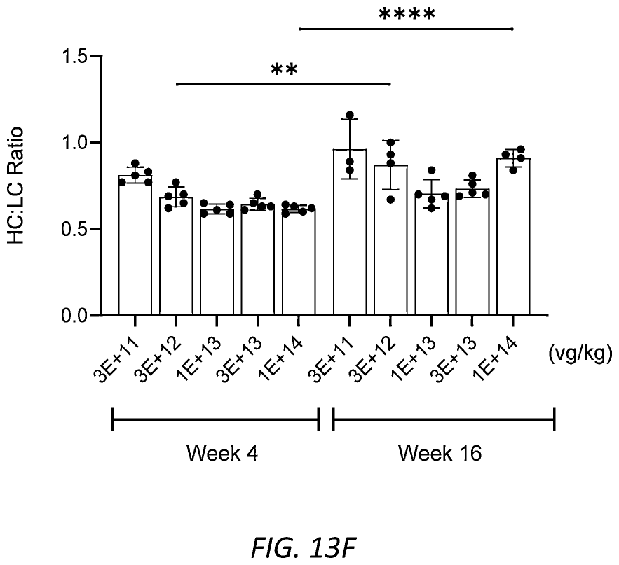

As shown in FIG. 13A, antibody levels were detectable 1 week post treatment at all doses tested. Sustained antibody levels were achieved for dose groups receiving 3e11 to 3e13 vg/kg of E55 vector, with anti-C5 antibody concentrations steadily increasing on weeks 1 through 5 and reaching a plateau thereafter. The steady state anti-C5 antibody concentration (CSS) was determined as the average of serum anti-C5 antibody level between weeks 5 to 16 for each dose group up to 3e13 vg/kg, and a linear correlation between CSS and dose level was found. For the 1e14 vg/kg dose group, supra-pharmacological anti-C5 antibody levels were achieved by week 1 (>20 mg/mL), followed by a decrease and stabilizing by weeks 9-16 to similar levels of antibody compared to the 3e13 vg/kg dose group.

A functional ex vivo hemolysis assay was performed at weeks 1, 3, 5, and 16, as described in Example 4, to determine doses that prevent ex vivo hemolysis and establish correlation between the level of serum anti-C5 antibody and ex vivo hemolysis. As shown in FIG. 13B, RBC hemolysis was abrogated by serum from all treated animals as early as 1 week post treatment.

Vector genome copy number in the liver of treated mice was determined at 4 and 16 weeks by droplet digital PCR (ddPCR) using a primer probe set targeting a region specific to the E55 vector and not present in the murine genome. As shown in FIG. 13C, a dose-dependent increase in VG concentrations was observed, along with stable VG levels achieved by week 4 for all doses except 1e14 vg/kg, where decrease in VGs was observed by week 16, correlating with loss of anti-C5 antibody CSS and transcript (see also, FIG. 13D and FIG. 13E).

The level of episomally-derived codon optimized heavy chain (CO-HC) and codon optimized light chain (CO-LC) transcripts and heavy chain to light chain ratio (HC:LC) was determined at 4- and 16-weeks post treatment. A housekeeping endogenous transcript (Gps1 mRNA) was measured to ensure adequate cDNA conversion and equal loading and to correlate levels to dose and vector genome copy number. As shown in FIG. 13D and FIG. 13E, dose-dependent increase in CO-HC and CO-LC transcripts, respectively, was observed, along with stable transcript levels achieved by week 4 for all doses except 1e14 vg/kg, correlating with VG levels determined as above. All HC:LC ratio averages were within 0.6 to 1, indicating similar levels of heavy and light chain transcripts (FIG. 13F).

### Example 7: In Vivo Expression of an Optimized Vector in a Humanized Liver Model

To evaluate the functional activity and durability of a bidirectional dual promoter vector in a humanized liver model, a four week study was carried out using C57BL/6J mice with the genotype Fah^{-/-} Rag2^{-/-} Il2rg^{-/-}, commonly referred to as FRG® Knockout mice (referred to herein as “HuLiv” mice). The mice were immunodeficient and lacked the tyrosine catabolic enzyme fumarylacetoacetate hydrolase (Fah). Ablation of mouse hepatocytes was induced by the withdrawal of the protective drug 2-(2-nitro-4-trifluoromethylbenzoyl)-1,3-cyclohexanedione (NTBC). The mice were then engrafted with human hepatocytes, and a urokinase-expressing adenovirus was administered to enhance repopulation of the human hepatocytes. Engraftment was sustained over the life of the animal with an appropriate regimen of CuRx™ Nitisinone (20-0026) and prophylactic treatment of SMX/TMP antibiotics (20-0037). Resulting HuLiv mouse livers were repopulated with >70% human hepatocytes. These HuLiv mice are described further in Azuma et al. (Nature Biotechnology. 25(8): 903-910 (2007)).

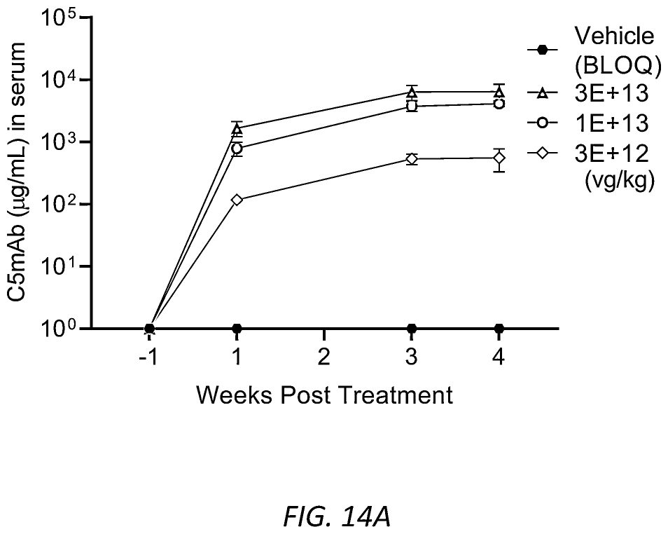

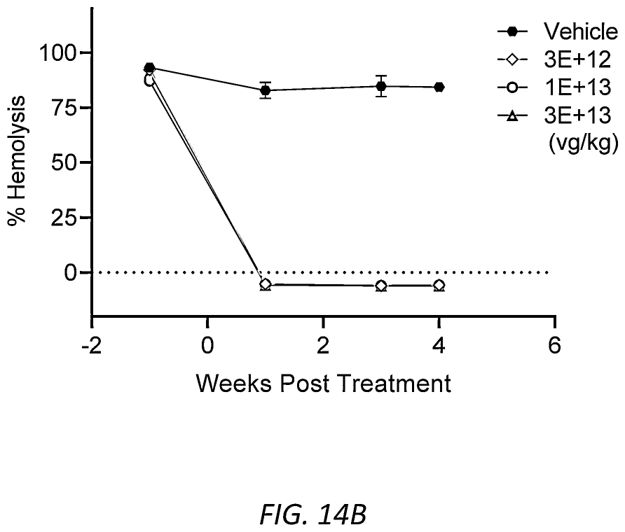

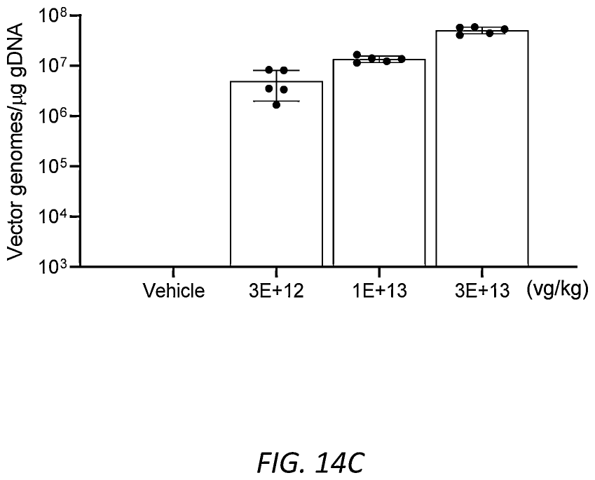

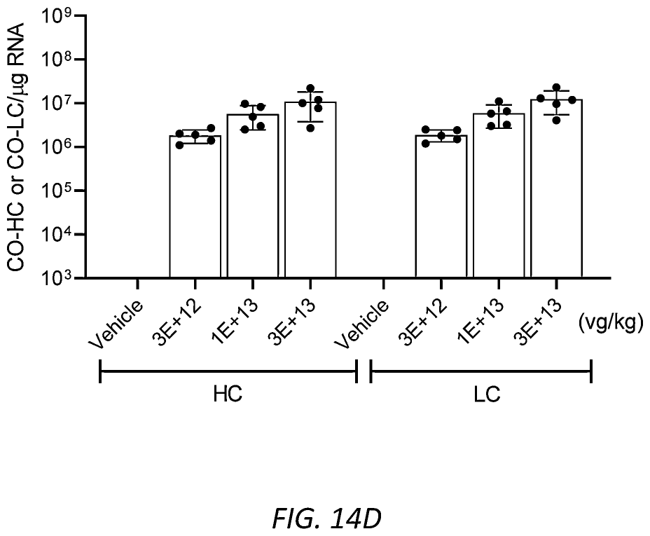

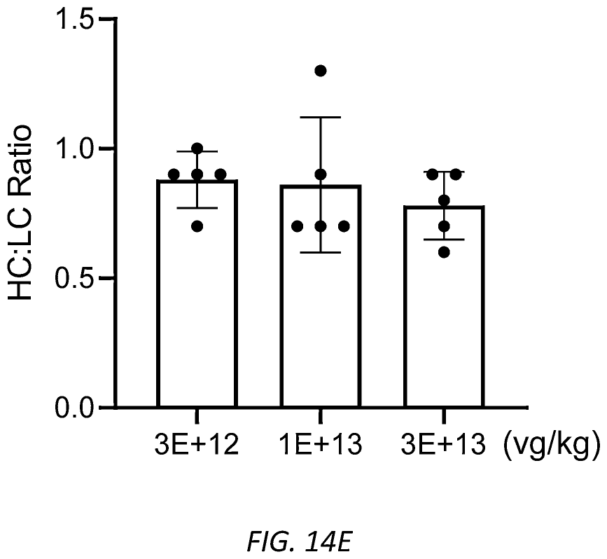

Anti-C5 antibody expression level and functional activity were measured in HuLiv mice dosed intravenously with 3e12, 1e13, and 3e13 vg/kg of vector E55 (packaged in an AAVHSC13 capsid), or with formulation buffer control (vehicle). 5 male mice were dosed in each group. None of the dose groups experienced significant loss of transplanted human hepatocytes or significant loss of weight over the study period. Serum samples were collected from each mouse at 1 and 3 weeks, and antibody levels were measured using a human IgG-specific ELISA, as described in Example 1. A terminal serum sample was collected from each mouse at 4 weeks. As shown in FIG. 14A, E55 packaged in AAVHSC13 capsid was able to successfully transduce human hepatocytes and produce detectable anti-C5 antibody levels 1 week post treatment at all doses tested. FIG. 14B shows that anti-C5 antibody produced in vivo by human hepatocytes is functional, demonstrated by complete inhibition of ex vivo hemolysis.

Vector genome copy number in the liver of treated mice was determined at 4 weeks by droplet digital PCR (ddPCR) using a primer probe set targeting a region specific to the E55 vector and not present in the murine genome. As shown in FIG. 14C, a dose-dependent increase in VG concentrations was observed.

The level of episomally-derived codon optimized heavy chain (CO-HC) and codon optimized light chain (CO-LC) transcripts and heavy chain to light chain ratio (HC:LC) was determined at 4 weeks post treatment. A housekeeping endogenous transcript (Gps1 mRNA) was measured to ensure adequate cDNA conversion and equal loading and to correlate levels to dose and vector genome copy number. As shown in FIG. 14D a dose-dependent increase in CO-HC and CO-LC transcripts was observed. HC:LC ratio averages were about 0.9 for all doses, indicating similar levels of heavy and light chain transcripts (FIG. 14E).

### Example 8: In Vivo Expression of an Optimized Vector in Immunocompetent Mice

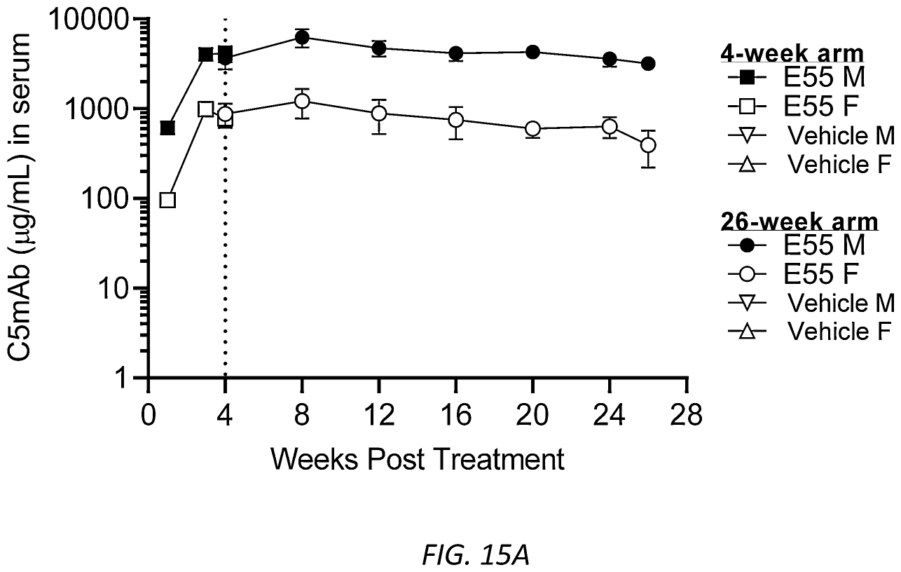

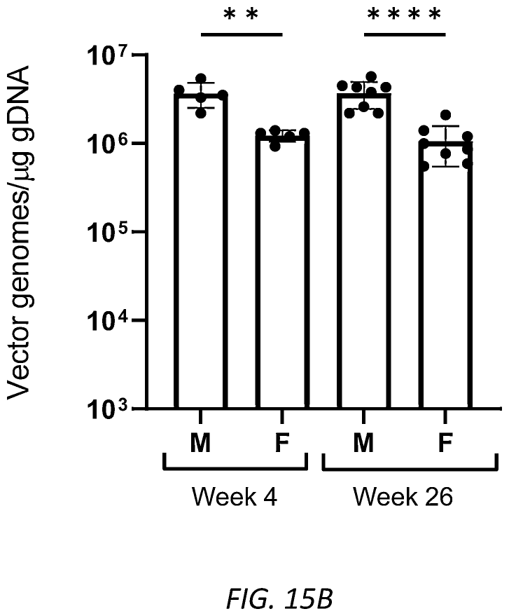

In order to investigate the long-term durability of a bidirectional dual promoter vector in an immunocompetent model, C57BL/6J mice were treated with a single 1e13 vg/kg dose of vector E55 packaged in an AAVHSC13 capsid (AAVHSC13-E55), or with formulation buffer (vehicle) as control. Two arms were studied: one arm with serum samples collected from each mouse at 1 and 3 weeks, and a terminal serum sample collected at 4 weeks (4 week arm); and a second arm with serum samples collected every month with a terminal serum sample collected at 26 weeks (26 week arm). 13 male and female mice were dosed in each group. FIG. 15A shows the anti-C5 antibody expression level measured in the mice of both groups out to 20 weeks post treatment. As shown, anti-C5 antibody expression was detectable at 1 week post treatment, and male mice expressed higher levels of anti-C5 antibody as compared to female mice. Durable anti-C5 antibody expression was observed for both sexes 20 weeks post treatment, suggesting tolerization of payload by vectorized delivery. FIG. 15B shows that for each sex, VG concentrations were stable throughout the duration of the study (26 weeks), with no statistical difference in VG concentrations between 4 weeks and 26 weeks post treatment. VG concentrations among animals of the same sex were comparable between 4 and 26 weeks, indicating that there was no loss of VGs over time. VG concentrations were greater in male mice compared to female mice.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. An isolated polynucleotide comprising from 5′ to 3′:
(a) a reverse complement human α1-antitrypsin (hAAT) promoter sequence, and a human hepatic control region 1 (hHCR1) sequence;
(b) a reverse complement hAAT promoter sequence that is at least 85% identical to SEQ ID NO:17, and an hHCR1 sequence that is at least 85% identical to SEQ ID NO:22;
(c) a reverse complement hAAT promoter sequence that is at least 85% identical to SEQ ID NO:17, and a reverse complement hHCR1 sequence that is at least 85% identical to SEQ ID NO:23;
(d) a reverse complement hAAT promoter sequence that is at least 85% identical to SEQ ID NO:18, and an hHCR1 sequence that is at least 85% identical to SEQ ID NO:22;
(e) a reverse complement hAAT promoter sequence that is at least 85% identical to SEQ ID NO:18, and a reverse complement hHCR1 sequence that is at least 85% identical to SEQ ID NO:23;
(f) a reverse complement hAAT promoter sequence that is at least 85% identical to SEQ ID NO:20, and an hHCR1 sequence that is at least 85% identical to SEQ ID NO:22;
(g) a reverse complement hAAT promoter sequence that is at least 85% identical to SEQ ID NO:20, and a reverse complement hHCR1 sequence that is at least 85% identical to SEQ ID NO:23;
(h) a reverse complement hAAT promoter sequence that is at least 85% identical to SEQ ID NO:17, and an hHCR1 sequence that is at least 85% identical to SEQ ID NO:24;
(i) a reverse complement hAAT promoter sequence that is at least 85% identical to SEQ ID NO:18, and an hHCR1 sequence that is at least 85% identical to SEQ ID NO:24;
(j) a reverse complement hAAT promoter sequence that is at least 85% identical to SEQ ID NO:18, and a reverse complement hHCR1 sequence that is at least 85% identical to SEQ ID NO:25;
(k) a reverse complement hAAT promoter sequence that is at least 85% identical to SEQ ID NO:20, and an hHCR1 sequence that is at least 85% identical to SEQ ID NO:24;
(l) a reverse complement hAAT promoter sequence that is at least 85% identical to SEQ ID NO:20, and a reverse complement hHCR1 sequence that is at least 85% identical to SEQ ID NO:25;
(m) an hHCR1 sequence that is at least 85% identical to SEQ ID NO:22, and a TTR promoter sequence that is at least 85% identical to SEQ ID NO:26;
(n) a reverse complement hHCR1 sequence that is at least 85% identical to SEQ ID NO:23, and a TTR promoter sequence that is at least 85% identical to SEQ ID NO:26;
(o) an hHCR1 sequence that is at least 85% identical to SEQ ID NO:24, and a TTR promoter sequence that is at least 85% identical to SEQ ID NO:26;
(p) a reverse complement hHCR1 sequence that is at least 85% identical to SEQ ID NO:25, and a TTR promoter sequence that is at least 85% identical to SEQ ID NO:26;
(q) a polyadenylation sequence, or a reverse complement thereof; and a TTR promoter sequence; or
an isolated polynucleotide, comprising:
(r) a nucleic acid sequence selected from the group consisting of SEQ ID NOs:19, 29-31, 44-59, 117-140, 168-172, 174-177, and 180-217;
(s) an hAAT promoter sequence consisting of a sequence that is at least 85% identical to SEQ ID NO:21, or 22, optionally wherein the hAAT promoter sequence consists of SEQ ID NO:21, or 22; or
(t) an hAAT promoter sequence comprising SEQ ID NO:19, optionally wherein the hAAT promoter sequence consists of SEQ ID NO:19.

2. The isolated polynucleotide of claim 1, comprising from 5′ to 3′:
(a) a reverse complement hAAT promoter sequence consisting of SEQ ID NO:17, and an hHCR1 sequence consisting of SEQ ID NO:22;
(b) a reverse complement hAAT promoter sequence consisting of SEQ ID NO:17, and a reverse complement hHCR1 sequence consisting of SEQ ID NO:23;
(c) a reverse complement hAAT promoter sequence consisting of SEQ ID NO:18, and an hHCR1 sequence consisting of SEQ ID NO:22;
(d) a reverse complement hAAT promoter sequence consisting of SEQ ID NO:18, and a reverse complement hHCR1 sequence consisting of SEQ ID NO:23;
(e) a reverse complement hAAT promoter sequence consisting of SEQ ID NO:20, and an hHCR1 sequence consisting of SEQ ID NO:22;
(f) a reverse complement hAAT promoter sequence consisting of SEQ ID NO:20, and a reverse complement hHCR1 consisting of SEQ ID NO:23;
(g) a reverse complement hAAT promoter sequence consisting of SEQ ID NO:17, and an hHCR1 sequence consisting of SEQ ID NO:24;
(h) a reverse complement hAAT promoter sequence consisting of SEQ ID NO:18, and an hHCR1 sequence consisting of SEQ ID NO:24;
(i) a reverse complement hAAT promoter sequence consisting of SEQ ID NO:18, and a reverse complement hHCR1 sequence consisting of SEQ ID NO:25;
(j) a reverse complement hAAT promoter sequence consisting of SEQ ID NO:20, and an hHCR1 sequence consisting of SEQ ID NO:24; or
(k) a reverse complement hAAT promoter sequence consisting of SEQ ID NO:20, and a reverse complement hHCR1 sequence consisting of SEQ ID NO:25,
optionally comprising a sequence selected from the group consisting of SEQ ID NOs: 36-40 and 72.

3. (canceled)

4. The isolated polynucleotide of claim 1, wherein (a) to (l) further comprises a transthyretin (TTR) promoter sequence positioned 3′ to the hHCR1 sequence or the reverse complement hHCR1 sequence, optionally wherein the TTR promoter sequence comprises a nucleic acid sequence that is at least 85% identical to SEQ ID NO:26.

5-6. (canceled)

7. The isolated polynucleotide of claim 1, comprising from 5′ to 3′:
(a) an hHCR1 sequence consisting of SEQ ID NO:22, and a TTR promoter sequence consisting of SEQ ID NO:26;
(b) a reverse complement hHCR1 sequence consisting of SEQ ID NO:23, and a TTR promoter sequence consisting of SEQ ID NO:26;
(c) an hHCR1 sequence consisting of SEQ ID NO:24, and a TTR promoter sequence consisting of SEQ ID NO:26; or
(d) a reverse complement hHCR1 sequence consisting of SEQ ID NO:25, and a TTR promoter sequence consisting of SEQ ID NO:26; or
comprising a sequence selected from the group consisting of SEQ ID NOs:41-43.

8. (canceled)

9. The isolated polynucleotide of claim 4, further comprising a polyadenylation sequence, or a reverse complement thereof, interposed between the hHCR1 sequence or the reverse complement hHCR1 sequence and the TTR promoter sequence, optionally wherein:
the polyadenylation sequence is a beta globin polyadenylation (BGpA) sequence;
the polyadenylation sequence is a beta globin polyadenylation (BGpA) sequence comprising a sequence that is at least 85% identical to SEQ ID NO:27; and/or
the isolated polynucleotide comprises a sequence that is at least 85% identical to a sequence selected from the group consisting of SEQ ID NOs:60-71 and 76-78.

10-13. (canceled)

14. The isolated polynucleotide of claim 1, wherein (a) to (l) further comprises a first intron element positioned 5′ to the reverse complement hAAT promoter sequence, optionally wherein:
the first intron element comprises a sequence that is at least 85% identical to SEQ ID NO:28, 29, 30, 31, or 32; and/or
the isolated polynucleotide further comprises a second intron element positioned 3′ to the TTR promoter sequence, optionally wherein:
the first and/or second intron element is a chimeric intron element;
the second intron element comprises a sequence that is at least 85% identical to SEQ ID NO:28, 29, 30, 31, or 32; and/or
the isolated polynucleotide comprises from 5′ to 3′:
(a) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:29;
(b) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:30;
(c) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:31; or
(d) a first intron element comprising a sequence that is at least 85% identical to SEQ ID NO:28; a nucleic sequence selected from the group consisting of SEQ ID NOs:60-71 and 76-78; and a second intron element comprising a sequence that is at least 85% identical to SEQ ID NO:32.

15-20. (canceled)

21. The isolated polynucleotide of claim 1, comprising a sequence that is at least 85% identical to a sequence selected from the group consisting of SEQ ID NOs:79-116.

22. (canceled)

23. The isolated polynucleotide of claim 1, wherein (a) to (l) further comprises: a reverse complement first coding sequence positioned 5′ of the reverse complement hAAT promoter sequence or the first intron element; and a second coding sequence positioned 3′ of the TTR promoter sequence of the second intron element, optionally wherein:
the isolated polynucleotide further comprises a reverse complement first polyadenylation sequence positioned 5′ to the reverse complement first coding sequence, optionally wherein the reverse complement first polyadenylation sequence comprises a sequence that is at least 85% identical to SEQ ID NO:33, 34, or 35; and/or
the isolated polynucleotide further comprises a second polyadenylation sequence positioned 3′ to the second coding sequence, optionally wherein the second polyadenylation sequence comprises a sequence that is at least 85% identical to SEQ ID NO:33, 34, or 35, optionally comprising from 5′ to 3′:
(a) a reverse complement first polyadenylation sequence comprising a sequence that is at least 85% identical to SEQ ID NO:33; a reverse complement first coding sequence; a nucleic sequence that is at least 85% identical to a nucleic acid sequence selected from the group consisting of SEQ ID NOs:60-71 and 76-116; a second coding sequence; and a second polyadenylation sequence comprising a sequence that is at least 85% identical to SEQ ID NO:35; or
(b) a reverse complement first polyadenylation sequence comprising a sequence that is at least 85% identical to SEQ ID NO:34; a reverse complement first coding sequence; a nucleic sequence that is at least 85% identical to a nucleic acid sequence selected from the group consisting of SEQ ID NOs:60-71 and 76-116; a second coding sequence; and a second polyadenylation sequence comprising a sequence that is at least 85% identical to SEQ ID NO:35.

24-29. (canceled)

30. The isolated polynucleotide of claim 23, wherein:
(a) the first coding sequence encodes an antibody heavy chain or an antigen-binding fragment thereof, and the second coding sequence encodes an antibody light chain or an antigen-binding fragment thereof; or
(b) the first coding sequence encodes an antibody light chain or an antigen-binding fragment thereof, and the second coding sequence encodes an antibody heavy chain or an antigen-binding fragment thereof, optionally wherein:
the antibody heavy chain comprises the amino acid sequence of SEQ ID NO:178, and the antibody light chain comprises the amino acid sequence of SEQ ID NO:179;
(a) the first coding sequence comprises a nucleic acid sequence selected from the group consisting of SEQ ID NOs:178-199, and the second coding sequence comprises a nucleic acid sequence selected from the group consisting of SEQ ID NOs:200-217;
(b) the first coding sequence comprises a nucleic acid sequence selected from the group consisting of SEQ ID NOs:200-217, and the second coding sequence comprises a nucleic acid sequence selected from the group consisting of SEQ ID NOs:178-199;
(c) the first coding sequence and the second coding sequence, respectively, comprise the nucleic acid sequences set forth in SEQ ID NOs:200 and 181, 191 and 209, 193 and 211, 195 and 213, 197 and 215, or 199 and 217; and/or
(d) the first coding sequence and the second coding sequence, respectively, comprise the nucleic acid sequences set forth in SEQ ID NOs:181 and 200, 209 and 191, 211 and 193, 213 and 195, 215 and 197, or 217 and 199.

31-33. (canceled)

34. The isolated polynucleotide of claim 30, wherein:
the first coding sequence and/or the second coding sequence comprises a signal sequence, optionally wherein the signal sequence comprises the amino acid sequence of SEQ ID NO:167 or 173; or
the first coding sequence and/or the second coding sequence comprise a nucleic acid sequence selected from the group consisting of SEQ ID NOs:168-172 and 174-177.

35-36. (canceled)

37. The isolated polynucleotide of claim 1, comprising from 5′ to 3′:
(a) SEQ ID NO:33; the reverse complement of SEQ ID NO:209; the reverse complement of SEQ ID NO:177, a nucleic sequence selected from the group consisting of SEQ ID NOs:60-71 and 76-116; SEQ ID NO:172; SEQ ID NO:191; and SEQ ID NO:35;
(b) SEQ ID NO:34; the reverse complement of SEQ ID NO:200; the reverse complement of SEQ ID NO:174, a nucleic sequence selected from the group consisting of SEQ ID NOs:60-71 and 76-116; SEQ ID NO:168; SEQ ID NO:181; and SEQ ID NO:35;
(c) SEQ ID NO:34; the reverse complement of SEQ ID NO:209; the reverse complement of SEQ ID NO:177, a nucleic sequence selected from the group consisting of SEQ ID NOs:60-71 and 76-116; SEQ ID NO:172; SEQ ID NO:191; and SEQ ID NO:35;
(d) SEQ ID NO:34; the reverse complement of SEQ ID NO:211; the reverse complement of SEQ ID NO:177, a nucleic sequence selected from the group consisting of SEQ ID NOs:60-71 and 76-116; SEQ ID NO:172; SEQ ID NO:193; and SEQ ID NO:35;
(e) SEQ ID NO:34; the reverse complement of SEQ ID NO:213; the reverse complement of SEQ ID NO:177, a nucleic sequence selected from the group consisting of SEQ ID NOs:60-71 and 76-116; SEQ ID NO:172; SEQ ID NO:195; and SEQ ID NO:35;
(f) SEQ ID NO:34; the reverse complement of SEQ ID NO:215; the reverse complement of SEQ ID NO:177, a nucleic sequence selected from the group consisting of SEQ ID NOs:60-71 and 76-116; SEQ ID NO:172; SEQ ID NO:197; and SEQ ID NO:35; or
(g) SEQ ID NO:34; the reverse complement of SEQ ID NO:217; the reverse complement of SEQ ID NO:177, a nucleic sequence selected from the group consisting of SEQ ID NOs:60-71 and 76-116; SEQ ID NO:172; SEQ ID NO:199; and SEQ ID NO:35.

38. The isolated polynucleotide of claim 1, wherein (a) to (l) further comprises a polyadenylation sequence, or a reverse complement thereof, positioned 3′ of the hHCR1 sequence or the reverse complement hHCR1 sequence, optionally wherein the polyadenylation sequence is a beta globin polyadenylation (BGpA) sequence, optionally wherein the BGpA sequence comprises a sequence that is at least 85% identical to SEQ ID NO:27.

39-40. (canceled)

41. The isolated polynucleotide of claim 1, wherein in (q):
the TTR promoter sequence comprises a nucleic acid sequence that is at least 85% identical to SEQ ID NO:26, optionally the TTR promoter sequence comprises SEQ ID NO:26, optionally the TTR promoter sequence consists of SEQ ID NO:26; and/or
the polyadenylation sequence is a beta globin polyadenylation (BGpA) sequence, optionally wherein the BGpA sequence comprises a sequence that is at least 85% identical to SEQ ID NO:27, optionally wherein the BGpA sequence comprises SEQ ID NO:27, optionally wherein the BGpA sequence consists of SEQ ID NO:27.

42-43. (canceled)

44. A polynucleotide that is the complement or the reverse complement of the polynucleotide of claim 1.

45. (canceled)

46. A recombinant adeno-associated virus (rAAV) genome comprising the polynucleotide of claim 1, optionally wherein:
the rAAV genome further comprises a 5′ inverted terminal repeat (5′ ITR) sequence, and a 3′ inverted terminal repeat (3′ ITR) sequence, optionally wherein the 5′ ITR nucleic acid sequence is at least 85% identical to the nucleic acid sequence set forth in SEQ ID NO:165, and/or the 3′ ITR nucleic acid sequence is at least 85% identical to the nucleic acid sequence set forth in SEQ ID NO:166;
the rAAV genome comprises a nucleic acid sequence that is at least 85% to the nucleic acid sequence set forth in SEQ ID NOs:141-164, or the complement thereof;
the rAAV genome is a single stranded rAAV genome; and/or
the rAAV genome is a self-complementary rAAV genome.

47-53. (canceled)

54. A nucleic acid vector comprising the isolated polynucleotide or rAAV genome of claim 46, optionally wherein the nucleic acid vector is a plasmid, a virus, or a DNA minimal vector.

55. An rAAV comprising: an AAV capsid comprising an AAV capsid protein; and an rAAV genome of claim 46, optionally wherein the capsid protein is selected from the group consisting of AAV1, AAV2, AAV3, AAV4, AAV5, AAV6, AAV7, AAV8, AAV9, AAVrh10, AAVrh74, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PHP.S.

56. (canceled)

57. The rAAV of claim 55, wherein:
the AAV capsid protein comprises an amino acid sequence that is at least 95% identical to the amino acid sequence of amino acids 203-736 of any one of SEQ ID NOs:1-15, optionally wherein:
(i) the amino acid in the capsid protein corresponding to amino acid 206 of SEQ ID NO:15 is C; the amino acid in the capsid protein corresponding to amino acid 296 of SEQ ID NO:15 is H; the amino acid in the capsid protein corresponding to amino acid 312 of SEQ ID NO:15 is Q, the amino acid in the capsid protein corresponding to amino acid 346 of SEQ ID NO:15 is A; the amino acid in the capsid protein corresponding to amino acid 464 of SEQ ID NO:15 is N; the amino acid in the capsid protein corresponding to amino acid 468 of SEQ ID NO:15 is S; the amino acid in the capsid protein corresponding to amino acid 501 of SEQ ID NO:15 is I; the amino acid in the capsid protein corresponding to amino acid 505 of SEQ ID NO:15 is R; the amino acid in the capsid protein corresponding to amino acid 590 of SEQ ID NO:15 is R; the amino acid in the capsid protein corresponding to amino acid 626 of SEQ ID NO:15 is G or Y; the amino acid in the capsid protein corresponding to amino acid 681 of SEQ ID NO:15 is M; the amino acid in the capsid protein corresponding to amino acid 687 of SEQ ID NO:15 is R; the amino acid in the capsid protein corresponding to amino acid 690 of SEQ ID NO:15 is K; the amino acid in the capsid protein corresponding to amino acid 706 of SEQ ID NO:15 is C; or, the amino acid in the capsid protein corresponding to amino acid 718 of SEQ ID NO:15 is G;
(ii) (a) the amino acid in the capsid protein corresponding to amino acid 626 of SEQ ID NO:15 is G, and the amino acid in the capsid protein corresponding to amino acid 718 of SEQ ID NO:15 is G; (b) the amino acid in the capsid protein corresponding to amino acid 296 of SEQ ID NO:15 is H, the amino acid in the capsid protein corresponding to amino acid 464 of SEQ ID NO:15 is N, the amino acid in the capsid protein corresponding to amino acid 505 of SEQ ID NO:15 is R, and the amino acid in the capsid protein corresponding to amino acid 681 of SEQ ID NO:15 is M; (c) the amino acid in the capsid protein corresponding to amino acid 505 of SEQ ID NO:15 is R, and the amino acid in the capsid protein corresponding to amino acid 687 of SEQ ID NO:15 is R; (d) the amino acid in the capsid protein corresponding to amino acid 346 of SEQ ID NO:15 is A, and the amino acid in the capsid protein corresponding to amino acid 505 of SEQ ID NO:15 is R; or (e) the amino acid in the capsid protein corresponding to amino acid 501 of SEQ ID NO:15 is I, the amino acid in the capsid protein corresponding to amino acid 505 of SEQ ID NO:15 is R, and the amino acid in the capsid protein corresponding to amino acid 706 of SEQ ID NO:15 is C;
(iii) the capsid protein comprises the amino acid sequence of amino acids 203-736 of any one of SEQ ID NOs:1-15; and/or
(iv) the AAV capsid protein is encoded by the nucleic acid sequence of nucleotides 607-2208 of SEQ ID NO:16;
the AAV capsid protein comprises an amino acid sequence that is at least 95% identical to the amino acid sequence of amino acids 138-736 of any one of SEQ ID NOs:1-15, optionally wherein:
(i) the amino acid in the capsid protein corresponding to amino acid 151 of SEQ ID NO:15 is R; the amino acid in the capsid protein corresponding to amino acid 160 of SEQ ID NO:15 is D; the amino acid in the capsid protein corresponding to amino acid 206 of SEQ ID NO:15 is C; the amino acid in the capsid protein corresponding to amino acid 296 of SEQ ID NO:15 is H; the amino acid in the capsid protein corresponding to amino acid 312 of SEQ ID NO:15 is Q, the amino acid in the capsid protein corresponding to amino acid 346 of SEQ ID NO:15 is A; the amino acid in the capsid protein corresponding to amino acid 464 of SEQ ID NO:15 is N; the amino acid in the capsid protein corresponding to amino acid 468 of SEQ ID NO:15 is S; the amino acid in the capsid protein corresponding to amino acid 501 of SEQ ID NO:15 is I; the amino acid in the capsid protein corresponding to amino acid 505 of SEQ ID NO:15 is R; the amino acid in the capsid protein corresponding to amino acid 590 of SEQ ID NO:15 is R; the amino acid in the capsid protein corresponding to amino acid 626 of SEQ ID NO:15 is G or Y; the amino acid in the capsid protein corresponding to amino acid 681 of SEQ ID NO:15 is M; the amino acid in the capsid protein corresponding to amino acid 687 of SEQ ID NO:15 is R; the amino acid in the capsid protein corresponding to amino acid 690 of SEQ ID NO:15 is K; the amino acid in the capsid protein corresponding to amino acid 706 of SEQ ID NO:15 is C; or, the amino acid in the capsid protein corresponding to amino acid 718 of SEQ ID NO:15 is G;
(ii) (a) the amino acid in the capsid protein corresponding to amino acid 626 of SEQ ID NO:15 is G, and the amino acid in the capsid protein corresponding to amino acid 718 of SEQ ID NO:15 is G; (b) the amino acid in the capsid protein corresponding to amino acid 296 of SEQ ID NO:15 is H, the amino acid in the capsid protein corresponding to amino acid 464 of SEQ ID NO:15 is N, the amino acid in the capsid protein corresponding to amino acid 505 of SEQ ID NO:15 is R, and the amino acid in the capsid protein corresponding to amino acid 681 of SEQ ID NO:15 is M; (c) the amino acid in the capsid protein corresponding to amino acid 505 of SEQ ID NO:15 is R, and the amino acid in the capsid protein corresponding to amino acid 687 of SEQ ID NO:15 is R; (d) the amino acid in the capsid protein corresponding to amino acid 346 of SEQ ID NO:15 is A, and the amino acid in the capsid protein corresponding to amino acid 505 of SEQ ID NO:15 is R; or (e) the amino acid in the capsid protein corresponding to amino acid 501 of SEQ ID NO:15 is I, the amino acid in the capsid protein corresponding to amino acid 505 of SEQ ID NO:15 is R, and the amino acid in the capsid protein corresponding to amino acid 706 of SEQ ID NO:15 is C;
(iii) the capsid protein comprises the amino acid sequence of amino acids 138-736 of any one of SEQ ID NOs:1-15; and/or
(iv) the AAV capsid protein is encoded by the nucleic acid sequence of nucleotides 414-2208 of SEQ ID NO:16; and/or
the AAV capsid protein comprises an amino acid sequence that is at least 95% identical to the amino acid sequence of amino acids 1-736 of any one of SEQ ID NOs:1-15, optionally wherein:
(i) the amino acid in the capsid protein corresponding to amino acid 2 of SEQ ID NO:15 is T; the amino acid in the capsid protein corresponding to amino acid 65 of SEQ ID NO:15 is I; the amino acid in the capsid protein corresponding to amino acid 68 of SEQ ID NO:15 is V; the amino acid in the capsid protein corresponding to amino acid 77 of SEQ ID NO:15 is R; the amino acid in the capsid protein corresponding to amino acid 119 of SEQ ID NO:15 is L; the amino acid in the capsid protein corresponding to amino acid 151 of SEQ ID NO:15 is R; the amino acid in the capsid protein corresponding to amino acid 160 of SEQ ID NO:15 is D; the amino acid in the capsid protein corresponding to amino acid 206 of SEQ ID NO:15 is C; the amino acid in the capsid protein corresponding to amino acid 296 of SEQ ID NO:15 is H; the amino acid in the capsid protein corresponding to amino acid 312 of SEQ ID NO:15 is Q; the amino acid in the capsid protein corresponding to amino acid 346 of SEQ ID NO:15 is A; the amino acid in the capsid protein corresponding to amino acid 464 of SEQ ID NO:15 is N; the amino acid in the capsid protein corresponding to amino acid 468 of SEQ ID NO:15 is S; the amino acid in the capsid protein corresponding to amino acid 501 of SEQ ID NO:15 is I; the amino acid in the capsid protein corresponding to amino acid 505 of SEQ ID NO:15 is R; the amino acid in the capsid protein corresponding to amino acid 590 of SEQ ID NO:15 is R; the amino acid in the capsid protein corresponding to amino acid 626 of SEQ ID NO:15 is G or Y; the amino acid in the capsid protein corresponding to amino acid 681 of SEQ ID NO:15 is M; the amino acid in the capsid protein corresponding to amino acid 687 of SEQ ID NO:15 is R; the amino acid in the capsid protein corresponding to amino acid 690 of SEQ ID NO:15 is K; the amino acid in the capsid protein corresponding to amino acid 706 of SEQ ID NO:15 is C; or, the amino acid in the capsid protein corresponding to amino acid 718 of SEQ ID NO:15 is G;
(ii) (a) the amino acid in the capsid protein corresponding to amino acid 2 of SEQ ID NO:15 is T, and the amino acid in the capsid protein corresponding to amino acid 312 of SEQ ID NO:15 is Q; (b) the amino acid in the capsid protein corresponding to amino acid 65 of SEQ ID NO:15 is I, and the amino acid in the capsid protein corresponding to amino acid 626 of SEQ ID NO:15 is Y; (c) the amino acid in the capsid protein corresponding to amino acid 77 of SEQ ID NO:15 is R, and the amino acid in the capsid protein corresponding to amino acid 690 of SEQ ID NO:15 is K; (d) the amino acid in the capsid protein corresponding to amino acid 119 of SEQ ID NO:15 is L, and the amino acid in the capsid protein corresponding to amino acid 468 of SEQ ID NO:15 is S; (e) the amino acid in the capsid protein corresponding to amino acid 626 of SEQ ID NO:15 is G, and the amino acid in the capsid protein corresponding to amino acid 718 of SEQ ID NO:15 is G; (f) the amino acid in the capsid protein corresponding to amino acid 296 of SEQ ID NO:15 is H, the amino acid in the capsid protein corresponding to amino acid 464 of SEQ ID NO:15 is N, the amino acid in the capsid protein corresponding to amino acid 505 of SEQ ID NO:15 is R, and the amino acid in the capsid protein corresponding to amino acid 681 of SEQ ID NO:15 is M; (g) the amino acid in the capsid protein corresponding to amino acid 505 of SEQ ID NO:15 is R, and the amino acid in the capsid protein corresponding to amino acid 687 of SEQ ID NO:15 is R; (h) the amino acid in the capsid protein corresponding to amino acid 346 of SEQ ID NO:15 is A, and the amino acid in the capsid protein corresponding to amino acid 505 of SEQ ID NO:15 is R; or (i) the amino acid in the capsid protein corresponding to amino acid 501 of SEQ ID NO:15 is I, the amino acid in the capsid protein corresponding to amino acid 505 of SEQ ID NO:15 is R, and the amino acid in the capsid protein corresponding to amino acid 706 of SEQ ID NO:15 is C;
(iii) the capsid protein comprises the amino acid sequence of amino acids 1-736 of SEQ ID NOs: 1-15; and/or
(iv) the AAV capsid protein is encoded by the nucleic acid sequence set forth in SEQ ID NO:16.

58-71. (canceled)

72. A pharmaceutical composition comprising a polynucleotide of claim 1.

73. A packaging system for preparation of an rAAV, wherein the packaging system comprises:
(a) a first nucleic acid sequence encoding one or more AAV Rep proteins;
(b) a second nucleic acid sequence encoding a capsid protein of the rAAV of claim 55; and
(c) a third nucleic acid sequence comprising an rAAV genome sequence of the rAAV of claim 55.

74-78. (canceled)

79. A method for recombinant preparation of an rAAV, the method comprising introducing the packaging system of claim 73 into a cell under conditions whereby the rAAV is produced.

80-82. (canceled)

83. A method of producing an antibody in a subject, the method comprising administering to the subject the rAAV of claim 55.

84. A method of treating a complement C5-associated disease in a subject in need thereof, the method comprising administering to the subject an effective amount of the rAAV of claim 55.

85-86. (canceled)
